SEQUENCE LISTING (88 sequences)

SEQ ID NO: 1 - DNA, 540 bp, Homo sapiens
aacggcggaggccacgccctctgtgaaagggcggggcatgcaaattcgaaatgaaagcccgggaacgccggaagaagcacgggtgtaagatttcccttttcaaaggcggagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgcatcgcttctcggccttttggctaagatcaagtgtagtatctgttcttatcagtttaatatctgatacgtcctctatccgaggacaatatattaaatggatttttggagcagggagatggaataggagcttgctccgtccactccacgcatcgacctggtattgcagtacctccaggaacggtgcaccccctccggggtacaacgtgtttcctaaaagtagagggaggtgagagacggtagcacctgcggggcggcttgcacgccgagtgcctgtgacgcgcc

SEQ ID NO: 2 - DNA, 1015 bp, Homo sapiens
gcgccttctacctcgctgtttcggttttcctggctcctcggcccttttctcccctgttgcagctgggagcggacgaagcgcgaagctgggattttttactgtctcctgaagaatttaacacaaacatggatatcagaccaaatcatacaatttatatcaacaatatgaatgacaaaattaaaaaggaagaattgaagagatccctatatgccctgttttctcagtttggtcatgtggtggacattgtggctttaaagaccatgaagatgagggggcaggcctttgtcatatttaaggaactgggctcatccacaaatgccttgagacagctacaaggatttccattttatggtaaaccaatgcgaatacagtatgcaaaaacagattcggatataatatcaaaaatgcgtggaacttttgctgacaaagaaaagaaaaaagaaaagaaaaaagccaaaactgtggaacagactgcaacaaccacaaacaaaaagcctggccagggaactccaaattcagctaatacccaaggaaattcaacaccaaatcctcaggtccctgattaccctccaaactatattttattccttaataacttaccagaagagactaatgagatgatgttatccatgctgtttaatcagttccctggcttcaaggaagtacgtctggtaccagggaggcatgacattgcttttgttgaatttgaaaatgatgggcaggctggagctgccagggatgctttacagggatttaagatcacaccgtcccatgctatgaagatcacctatgccaagaaataacatttgggatagtcgtctttaaaagacttggtgttatttacagtgtttgttttgataacatttggctgggtcattttaatagttagagatgaggaggagtaaaagtgaaatttttgtgaaggacttaaattatccagtgtttctttagccttggtgaactatgaaatacgaaggccttaattttgtacaataaacttttatttgtattctgt

SEQ ID NO: 3 - protein, 225 aa, Homo sapiens
MDIRPNHTIYINNMNDKIKKEELKRSLYALFSQFGHVVDIVALKTMKMRGQAFVIFKELGSSTNALRQLQGFPFYGKPMRIQYAKTDSDIISKMRGTFADKEKKKEKKKAKTVEQTATTTNKKPGQGTPNSANTQGNSTPNPQVPDYPPNYILFLNNLPEETNEMMLSMLFNQFPGFKEVRLVPGRHDIAFVEFENDGQAGAARDALQGFKITPSHAMKITYAKK

SEQ ID NO: 4 - protein, 227 aa, Artificial Sequence
  Description of Artificial Sequence Synthetic U2 snRNP B consensus sequence
MDIRPNHTIYINNMNDKIKKEELKRSLYALFSQFGHVVDIVALKTMKMRGQAFVIFKELGSSTNALRQLQGFPFYGKPMRIQYAKTXSDIISKMRGXFADKEKKXEKKKAKTXEQXAXXXXKKPGXGXXXXAXXXXXXXXXXXPXVPDYPPNYILFLNNLPEETNXMMLSMLFNQFPGFKEVXLVPGRHDIAFVEFEXDGQAGAAXDALQGFKITPSHAMKITYAKK

SEQ ID NO: 5 - DNA, 4259 bp, Homo sapiens
atggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaagacccctgatcctaaaatgaatgttaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggcctagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggcaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagttcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaaaaaaaaaaaaaaaaa

SEQ ID NO: 6 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNVRTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGLEIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 7 - protein, 1319 aa, Artificial Sequence
  Description of Artificial Sequence Synthetic SAP155 consensus sequence
XXXXXXXXXXIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNXRTYMDVMREQHLTKEEREIRQQLAEKAKAGELKVVNGAAASQPPSKRKRRWDQTADQTPGATPKKLSSWDXXXXXXXXXXXTPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGXEIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVXXXXXXXXXXXXXXXXNYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNXXXVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYXLDYIL

SEQ ID NO: 8 - DNA, 2839 bp, Homo sapiens
ctcccaaagcagaattgcagctgccgccgccgccacctccaggccactatggcgcctggggctgcccaggagcttcaggccaagttggcagagatcggagctccgatccagggtaatcgcgaggagctggtggagcggctgcagagctacacccgccagactggcatcgtgctgaatcggccggttttgagaggggaagatggggacaaagccgctccacctcccatgtcggcacagctccctggaattcccatgccaccaccacctttgggactcccccctctgcagcctcctccgccacccccaccacctccaccaggccttggccttggctttcctatggcccacccaccaaatttggggcccccgcctcctctccgtgtgggtgagccagtggcactgtcagaggaggagcggctgaagttggctcagcagcaggcggcattgctgatgcagcaggaggagcgtgccaagcagcagggagatcattcgctgaaggaacatgagctcttggagcagcagaagcgggcagctgtgttactggagcaggaacgacagcaggagattgccaagatgggcaccccagtccctcggcccccacaagacatgggccagattggtgtgcgcactcctctgggtcctcgagtagctgctccagtgggcccagtgggccccactcctacagttttgcccatgggagcccctgttccccggcctcgtggtcccccaccgccccctggagatgagaacagagagatggatgacccctctgtgggccccaagatcccccaggctttggagaagatcctgcagctgaaggagagccgccaggaagagatgaattctcagcaggaggaagaggaaatggaaacagatgctcgctcgtccctgggccagtcagcgtcagagactgaggaggacacagtgtccgtatctaaaaaggagaaaaaccggaagcgtaggaaccgaaagaagaagaaaaagccccagcgggtgcgaggggtgtcctctgagagctctggggaccgggagaaagactcaacccggtcccgtggctctgattccccagcagctgatgttgagattgagtatgtgactgaagaacctgaaatttacgagcccaactttatcttctttaagaggatctttgaggcttttaagctcactgatgatgtgaagaaggagaaagagaaagagccagagaaacttgacaaactggagaactctgcagcccccaagaagaagggatttgaagaggagcacaaggacagtgatgatgacagcagtgatgacgagcaggaaaagaagccagaagcccccaagctgtccaagaagaagttgcgccgaatgaaccgcttcactgtggctgaactcaagcagctggtggctcggcccgatgtcgtggagatgcacgatgtgacagcgcaggaccctaagctcttggttcacctcaaggccactcggaactctgtgcctgtgccacgccactggtgttttaagcgcaaatacctgcagggcaaacggggcattgagaagccccccttcgagctgccagacttcatcaaacgcacaggcatccaggagatgcgagaggccctgcaggagaaggaagaacagaagaccatgaagtcaaaaatgcgagagaaagttcggcctaagatgggcaaaattgacatcgactaccagaaactgcatgatgccttcttcaagtggcagaccaagccaaagctgaccatccatggggacctgtactatgaggggaaggagttcgagacacgactgaaggagaagaagccaggagatctgtctgatgagctaaggatttccttggggatgccagtaggaccaaatgcccacaaggtccctcccccatggctgattgccatgcagcgatatggaccacccccatcgtatcccaacctgaaaatccctgggctgaactcgcccatccctgagagctgttcctttgggtaccatgctggtggctggggcaaacctccagtggatgagactgggaaaccgctctatggggacgtgtttggaaccaatgctgctgaatttcagaccaagactgaggaagaagagattgatcggaccccttggggggaactggaaccatctgatgaagaatcctcagaagaagaggaagaggaagaaagtgatgaagacaaaccagatgagacaggctttattacccctgcagacagtggccttatcactcctggaggcttttcatcagtgcctgctggaatggagacccctgaactcattgagctgaggaagaagaagattgaggaggcgatggacggaagtgagacacctcagctcttcactgtgttgccagagaagagaacagccactgttggaggggccatgatgggatcaacccacatttatgacatgtccacggttatgagccggaagggcccggctcctgagctgcaaggtgtggaagtggcgctggcgcctgaagagttggagctggatcctatggccatgacccagaagtatgaggagcatgtgcgggagcagcaggctcaagtagagaaggaggacttcagtgacatggtggctgagcacgctgccaaacagaagcaaaaaaaacggaaagctcagccccaggacagccgtgggggcagcaagaaatataaggagttcaagttttaggtcccctcacactagccctttttttggccctacgtctggatgcctgggcttcacacaagaaccacctctcccgcagttcccaaggacttgtcatttcatgttcttattttagacctgttttgtaaataaagctgtttcccaaggaaagagatgaaaaaaaaaaaaaaaaaaa

SEQ ID NO: 9 - protein, 872 aa, Homo sapiens
MAPGAAQELQAKLAEIGAPIQGNREELVERLQSYTRQTGIVLNRPVLRGEDGDKAAPPPMSAQLPGIPMPPPPLGLPPLQPPPPPPPPPPGLGLGFPMAHPPNLGPPPPLRVGEPVALSEEERLKLAQQQAALLMQQEERAKQQGDHSLKEHELLEQQKRAAVLLEQERQQEIAKMGTPVPRPPQDMGQIGVRTPLGPRVAAPVGPVGPTPTVLPMGAPVPRPRGPPPPPGDENREMDDPSVGPKIPQALEKILQLKESRQEEMNSQQEEEEMETDARSSLGQSASETEEDTVSVSKKEKNRKRRNRKKKKKPQRVRGVSSESSGDREKDSTRSRGSDSPAADVEIEYVTEEPEIYEPNFIFFKRIFEAFKLTDDVKKEKEKEPEKLDKLENSAAPKKKGFEEEHKDSDDDSSDDEQEKKPEAPKLSKKKLRRMNRFTVAELKQLVARPDVVEMHDVTAQDPKLLVHLKATRNSVPVPRHWCFKRKYLQGKRGIEKPPFELPDFIKRTGIQEMREALQEKEEQKTMKSKMREKVRPKMGKIDIDYQKLHDAFFKWQTKPKLTIHGDLYYEGKEFETRLKEKKPGDLSDELRISLGMPVGPNAHKVPPPWLIAMQRYGPPPSYPNLKIPGLNSPIPESCSFGYHAGGWGKPPVDETGKPLYGDVFGTNAAEFQTKTEEEEIDRTPWGELEPSDEESSEEEEEEESDEDKPDETGFITPADSGLITPGGFSSVPAGMETPELIELRKKKIEEAMDGSETPQLFTVLPEKRTATVGGAMMGSTHIYDMSTVMSRKGPAPELQGVEVALAPEELELDPMAMTQKYEEHVREQQAQVEKEDFSDMVAEHAAKQKQKKRKAQPQDSRGGSKKYKEFKF

SEQ ID NO: 10 - protein, 889 aa, Artificial Sequence
  Description of Artificial Sequence Synthetic SAP145 consensus sequence
XXPXPPPGHYGAWAAQELQAXLAEIGAPIQGXREELVERLQXYTRQTGIVLNRPVLRGEDGDKAAPPPMSAQLXGIPMPPPPXGLPPLQPPPPPPPPPPGLGLGFPMAXXXXPPNLGPPPPLRVGEPVALSEEERLKLAQQQAALLMQQEERAKQQGDHSLKEHELLEQXKRAAVLXEQERQQEIAKMGTXVPRPPQDMGQIGVRXXXXPXXXXXXXPVXPTPTVLPMGAPVPRPRGPPPPPGDENREMDDPSVGPKIPQALEKILQLKESRQEEMNSQQEEEEMETDXRSSLGQSASETEEDTVSXSKKEKNRKRRNRKKKKKPQRVRXSSESXGDREKXSXRSRGSDXPAADVEIEYVTEEPEIYEPNFIFFKRIFEAFKLTDDVKKEKEKEPEKLDKXEXSXXPKKKGFEEEHKDSDDDSSDDEQEKKPEAPKLSKKKLRRMNRFTVAELKQLVAXPDVVEMHDVTAQDPKLLVHLKATRNSVPVPRXWCFKRKYLQGKRGIEKPPFELPDFIKRTGIQEMREALQEKEEQKTMKSKMREKVRPKMGKIDIDYQKLHDAFFKWQTKPKLTIHGDLYYEGKEFETRLKEKKPGDLSDELRISLGMPVGPNAHKVPPPWLIAMQRYGPPPSYPNLKIPGLNSPIPEXXSFGYHAGGWGKPPVDETGKPLYGDVFGTNAAEFQTKTEEEEIDRTPWGELEPSDEESSEEEEEEESDEDKPDETGFITPADSGLITPGGFSSVPAGMETPELIELRKKKIEEAMDGSETPQLFTVLPEXRTATVGGAMMGSTHIYDMSTVXXXXXXXXXXXXXPELQGVEVALAPEELELDPMAMTQKYEEHVREQQAQVEKEDFSDMVAEHAAKQKQKKXKAQPQDSRGGSKKYKEFKF

SEQ ID NO: 11 - DNA, 1525 bp, Homo sapiens
attcggcatgagggggtcacgtggtggctgggccggggaaatggcggcttcaggagagagcgggacttcaggcggcggaggcagcaccgaggaagcatttatgaccttctacagtgaggtgaaacaaatagagaagagagactcggttctaacttcgaaaaatcagattgaaagactgacccgtcctggttcctcttacttcaatttgaacccatttgaggttcttcagatagatcctgaagttacagatgaagaaataaaaaagaggtttcggcagttatccatcttggtgcatcctgacaaaaatcaagatgatgctgacagagcacaaaaggcttttgaagctgtggacaaagcttacaagttgctactggatcaggagcaaaagaagagggccctggatgtaattcaggcaggaaaagaatacgtggaacacactgtgaaagagcgaaaaaaacaattaaagaaggaaggaaaacctacaattgtagaggaggatgatcctgagctgttcaaacaagctgtatataaacagacaatgaaactctttgcagagctggaaattaaaaggaaagagagagaagccaaagagatgcatgaaaggaaacgacaaagggaagaagagattgaagctcaagaaaaagccaaacgggaaagagagtggcagaaaaactttgaggaaagtcgagatggtcgtgtggacagctggcgaaacttccaagccaatacgaaggggaagaaagagaagaaaaatcggaccttcctgagaccaccgaaagtaaaaatggagcaacgtgagtgaccgcccaaggtcacaggcacagaacctttcccctgctatctcccttcctgcttcgaaggactcattctttcctcccacttccaccccaacatagagtagtatttgctttttagtccattttgttttcaatacgatttaatatcgatcagagtaattcttttgtacattgaaatgaggggcttggtttaaaaaaagacctttccctctccctgcccctagaacaaccagtattagaaggtgccaccattggtgctgccttctcttcccacagcctgtaactcagtgttttgtacttcactgaattgtgatggttagaaacttcgtggatagtttgtggaaatcatccaattaaacatactgcttaaaacagtgttgctgtgacttcagagacaagcctggaaggggcaccttaggaagccccttcgcttcagttgctcgcttctgggtgtgctcccttcgaaggcccagataagacagggaacacttgtgagcacacagagcagcatctgatgccctgtggtgtttggcatgtgccccctgtctactgaccaatcagtgtggcatgaggcccacgccacccaaacctttcactttccaaagagctagccgtcctccacccagtaccatgtcctagcctgtctgcatttgttagtggtaatattctttatgtataataaatttttatacccaaaaaaaaaaaaaaaaaaaaaaaaaaaaaaaaaa

SEQ ID NO: 12 - protein, 264 aa, Homo sapiens
MRGSRGGWAGEMAASGESGTSGGGGSTEEAFMTFYSEVKQIEKRDSVLTSKNQIERLTRPGSSYFNLNPFEVLQIDPEVTDEEIKKRFRQLSILVHPDKNQDDADRAQKAFEAVDKAYKLLLDQEQKKRALDVIQAGKEYVEHTVKERKKQLKKEGKPTIVEEDDPELFKQAVYKQTMKLFAELEIKRKEREAKEMHERKRQREEEIEAQEKAKREREWQKNFEESRDGRVDSWRNFQANTKGKKEKKNRTFLRPPKVKMEQRE

SEQ ID NO: 13 - protein, 264 aa, Artificial Sequence
  Description of Artificial Sequence Synthetic SPF31 consensus sequence
XXXXXXXXXXXXAAXGESGXSXGGGSXXXXXXXXXXEVXQIEKRDSVLTSKNQIERLTRPGSSYFNLNPFEVLQIDPEVTDEEIKKXFRQLSILVHPDKNQDDADRAQKAFEAVDKAYKLLLDQEQKKRALDVIQAGKEYVEHTVKERKKQLKKEGKPTXVXEDDPELFKQAVYKQTMKLFAELEIKRKEREAKEMHERKRQREEEIEAQEKAKREREWQKNFEESRDGRXDSWRNXXXXXKXXKXXKXXXKXXXXXXXXXXXX

SEQ ID NO: 14 - DNA, 4595 bp, Homo sapiens
gcatgctcgacgcgccatgtgatggatatctgcagaattcggcttactcactatagggctcgagcggccgcccgggcaggtcgccagtatgttggagttggtggtggcttaagttttgaagggaggtagcatccgttggatatccacaccatccttctcgctgcaggctttcttggactccgtactgttggtgtaaccaaggcctggaggtctgggtggctcaggtttcctgcagccatgtttctgtacaacttaaccttgcagagagccactggcatcagctttgccattcatggaaacttttctggaaccaaacaacaagaaattgttgtttcccgtgggaagatcttggagctgcttcgcccagaccccaacactggcaaagtacataccctactcactgtggaagtattcggtgttatccggtcactcatggcctttaggctgacaggtggcaccaaagactacattgtagttggcagtgactctggtcgaattgttattttggaataccagccatctaagaatatgtttgagaagattcaccaagaaacctttggcaagagtggatgccgtcgcatcgttcctggccagttcttagctgtggatcccaaagggcgagccgttatgattagtgccattgagaaacagaaattggtgtatattttgaacagagatgctgcagcccgacttaccatttcatctcccctggaagcccacaaagcaaacactttagtgtatcatgtagttggagtagatgtcggatttgaaaatccaatgtttgcttgtctggaaatggattatgaggaagcaggcaatgatccaacaggggaagcagcagctaatacccagcagacacttactttctatgagctagaccttggtttaaatcatgtggtccgaaaatacagtgaacctttggaggaacacggcaacttccttattacagttccaggagggtcagatggtccaagtggagtactgatctgctctgaaaactatattacttacaagaactttggtgaccagccagatatccgctgtccaattcccaggaggcggaatgacctggatgaccctgaaagaggaatgatttttgtctgctctgcaacccataaaaccaaatcgatgttcttcttttgggctcaaactgagcagggagatatctttaagatcactttggagacagatgaagatatggttactgagatccggctcaaatattttgatactgtacccgttgctgctgccatgtgtgtgcttaaaacagggttcctttttgtagcatcagaatttggaaaccattacttatatcaaattgcacatcttggagatgatgatgaagaacctgagttttcatcagccatgcctctggaagaaggagacacattcttttttcagccaagaccacttaaaaaccttgtgctggttgatgagttggacagcctctctcccattctgttttgccagatagctgatctggccaatgaagatactccacagttgtatgtggcctgtggtaggggaccccgatcatctctgagagtcctaagacatggacttgaggtgtcagaaatggctgtttctgagctacctggtaaccccaacgctgtctggacagtgcgtcgacacattgaagatgagtttgatgcctacatcattgtgtctttcgtgaatgccaccctagtgttgtccattggagaaactgtagaagaagtgactgactctgggttcctggggaccaccccgaccttgtcctgctccttattaggagatgatgccttggtgcaggtctatccagatggcattcggcacatacgagcagacaagagagtcaatgagtggaagacccctggaaagaaaacaattgtgaagtgtgcagtgaaccagcgacaagtggtgattgccctgacaggaggagagctggtctatttcgagatggatccttcaggacagctgaatgagtacacagaacggaaggagatgtcagcagatgtggtgtgcatgagtctggccaatgtaccccctggagagcagcggtctcgcttcctggctgtggggcttgtggacaacactgtcagaatcatctccctggatccctcagactgtttgcaacctctaagcatgcaggctctcccagcccagcctgagtccttgtgtatcgtggaaatgggtgggactgagaagcaggatgagctgggtgagaggggctcgattggcttcctatacctgaatattgggctacagaacggtgtgctgctgaggactgtcttggaccctgtcactggggatttgtctgatactcgcactcggtacctggggtcccgtcctgtgaagctcttccgagtccgaatgcaaggccaggaggcagtattggccatgtcaagccgctcatggttgagctattcttaccaatctcgcttccatctcaccccactgtcttacgagacactggaatttgcatcgggttttgcctcggaacagtgtcccgagggcattgtggccatctccaccaacaccctacggattttggcattagagaagctcggtgctgtcttcaatcaagtagccttcccactgcagtacacacccaggaaatttgtcatccaccctgagagtaacaaccttattatcattgaaacggaccacaatgcctacactgaggccacgaaagctcagagaaagcagcagatggcagaggaaatggtggaagcagcaggggaggatgagcgggagctggccgcagagatggcagcagcattcctcaatgaaaacctccctgaatccatctttggagctcccaaggctggcaatgggcagtgggcctctgtgatccgagtgatgaatcccattcaagggaacacactggaccttgtccagctggaacagaatgaggcagcttttagtgtggctgtgtgcaggttttccaacactggtgaagactggtatgtgctggtgggtgtggccaaggacctgatactaaacccccgatctgtggcagggggcttcgtctatacttacaagcttgtgaacaatggggaaaaactggagtttttgcacaagactcctgtggaagaggtccctgctgctattgccccattccaggggagggtgttgattggtgtggggaagctgttgcgtgtctatgacctgggaaagaagaagttactccgaaaatgtgagaataagcatattgccaattatatctctgggatccagactattggacatagggtaattgtatctgatgtccaagaaagtttcatctgggttcgctacaagcgtaatgaaaaccagcttatcatctttgctgatgatacctacccccgatgggtcactacagccagcctcctggactatgacactgtggctggggcagacaagtttggcaacatatgtgtggtgaggctcccacctaacaccaatgatgaagtagatgaggatcctacaggaaacaaagccctgtgggaccgtggcttgctcaatggggcctcccagaaggcagaggtgatcatgaactaccatgtcggggagacggtgctgtccttgcagaagaccacgctgatccctggaggctcagaatcacttgtctataccaccttgtctggaggaattggcatccttgtgccattcacgtcccatgaggaccatgacttcttccagcatgtggaaatgcacctgcggtctgaacatccccctctctgtgggcgggaccacctcagctttcgctcctactacttccctgtgaagaatgtgattgatggagacctctgtgagcagttcaattccatggaacccaacaaacaaaagaacgtctctgaagaactggaccgaaccccacccgaagtgtccaagaaactcgaggatatccggacccgctacgccttctgagccctcctttcccggtggggcttgccagagactgtgtgttttgtttcccccaccaccatcactgccacctggcttctgccatgtggcaggagggtgactggataattaagactgcattatgaaagtcaacagctctttcccctcagctcttctcctggaatgactggcttcccctcaaattggcactgagatttgctacacttctccccacctggtacatgatacatgaccccaggttccagtgtagaacctgagtcccccattccccaaagccatccctgcattgatatgtcttgactctcctgtctacttttgcacacacccttaatttttaattggttttcttgtaaatacagttttgtacaatgttatctctgtgggaggaaggaggcaggctgtggtgggactgggtagggtatagtatcactcctgagttccactgctctagaatctaaccagaaatagaaacctagtttttaaggtgactggcatccatgtgtcttgttctggagatgaggatgtaggtgggaggtttgaacccaagttagagcaggaagaactgagtagactccttccttccagataccgacttggacttgcggcactctgtggctccccacccccaggtctgtggtggtttctttgttttttcctggttctttttgctgtgctgatgaaacatgacctcaataaccatgtgtata

SEQ ID NO: 15 - protein, 1217 aa, Homo sapiens
MFLYNLTLQRATGISFAIHGNFSGTKQQEIVVSRGKILELLRPDPNTGKVHTLLTVEVFGVIRSLMAFRLTGGTKDYIVVGSDSGRIVILEYQPSKNMFEKIHQETFGKSGCRRIVPGQFLAVDPKGRAVMISAIEKQKLVYILNRDAAARLTISSPLEAHKANTLVYHVVGVDVGFENPMFACLEMDYEEAGNDPTGEAAANTQQTLTFYELDLGLNHVVRKYSEPLEEHGNFLITVPGGSDGPSGVLICSENYITYKNFGDQPDIRCPIPRRRNDLDDPERGMIFVCSATHKTKSMFFFWAQTEQGDIFKITLETDEDMVTEIRLKYFDTVPVAAAMCVLKTGFLFVASEFGNHYLYQIAHLGDDDEEPEFSSAMPLEEGDTFFFQPRPLKNLVLVDELDSLSPILFCQIADLANEDTPQLYVACGRGPRSSLRVLRHGLEVSEMAVSELPGNPNAVWTVRRHIEDEFDAYIIVSFVNATLVLSIGETVEEVTDSGFLGTTPTLSCSLLGDDALVQVYPDGIRHIRADKRVNEWKTPGKKTIVKCAVNQRQVVIALTGGELVYFEMDPSGQLNEYTERKEMSADVVCMSLANVPPGEQRSRFLAVGLVDNTVRIISLDPSDCLQPLSMQALPAQPESLCIVEMGGTEKQDELGERGSIGFLYLNIGLQNGVLLRTVLDPVTGDLSDTRTRYLGSRPVKLFRVRMQGQEAVLAMSSRSWLSYSYQSRFHLTPLSYETLEFASGFASEQCPEGIVAISTNTLRILALEKLGAVFNQVAFPLQYTPRKFVIHPESNNLIIIETDHNAYTEATKAQRKQQMAEEMVEAAGEDERELAAEMAAAFLNENLPESIFGAPKAGNGQWASVIRVMNPIQGNTLDLVQLEQNEAAFSVAVCRFSNTGEDWYVLVGVAKDLILNPRSVAGGFVYTYKLVNNGEKLEFLHKTPVEEVPAAIAPFQGRVLIGVGKLLRVYDLGKKKLLRKCENKHIANYISGIQTIGHRVIVSDVQESFIWVRYKRNENQLIIFADDTYPRWVTTASLLDYDTVAGADKFGNICVVRLPPNTNDEVDEDPTGNKALWDRGLLNGASQKAEVIMNYHVGETVLSLQKTTLIPGGSESLVYTTLSGGIGILVPFTSHEDHDFFQHVEMHLRSEHPPLCGRDHLSFRSYYFPVKNVIDGDLCEQFNSMEPNKQKNVSEELDRTPPEVSKKLEDIRTRYAF

SEQ ID NO: 16 - DNA, 2613 bp, Homo sapiens
cttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattc

SEQ ID NO: 17 - protein, 793 aa, Homo sapiens
MPAGPVQAVPPPPPVPTEPKQPTEEEASSKEDSAPSKPVVGIIYPPPEVRNIVDKTASFVARNGPEFEARIRQNEINNPKFNFLNPNDPYHAYYRHKVSEFKEGKAQEPSAAIPKVMQQQQQTTQQQLPQKVQAQVIQETIVPKEPPPEFEFIADPPSISAFDLDVVKLTAQFVARNGRQFLTQLMQKEQRNYQFDFLRPQHSLFNYFTKLVEQYTKILIPPKGLFSKLKKEAENPREVLDQVCYRVEWAKFQERERKKEEEEKEKERVAYAQIDWHDFVVVETVDFQPNEQGNFPPPTTPEELGARILIQERYEKFGESEEVEMEVESDEEDDKQEKAEEPPSQLDQDTQVQDMDEGSDDEEEGQKVPPPPETPMPPPLPPTPDQVIVRKDYDPKASKPLPPAPAPDEYLVSPITGEKIPASKMQEHMRIGLLDPRWLEQRDRSIREKQSDDEVYAPGLDIESSLKQLAERRTDIFGVEETAIGKKIGEEEIQKPEEKVTWDGHSGSMARTQQAAQANITLQEQIEAIHKAKGLVPEDDTKEKIGPSKPNEIPQQPPPPSSATNIPSSAPPITSVPRPPTMPPPVRTTVVSAVPVMPRPPMASVVRLPPGSVIAPMPPIIHAPRINVVPMPPSAPPIMAPRPPPMIVPTAFVPAPPVAPVPAPAPMPPVHPPPPMEDEPTSKKLKTEDSLMPEEEFLRRNKGPVSIKVQVPNMQDKTEWKLNGQVLVFTLPLTDQVSVIKVKIHEATGMPAGKQKLQYEGIFIKDSNSLAYYNMANGAVIHLALKERGGRKK

SEQ ID NO: 18 - DNA, 1395 bp, Homo sapiens
atggacttccagcatcgccccgggggcaagaccgggagcgggggcgtggcctcctcctccgagagcaaccgtgaccgcagggagccgctccggcagctggccctggagaccatcgacatcaacaaggacccgtacttcatgaagaaccacctgggctcctatgaatgcaaactctgcctgacacttcacaacaatgaggggagctacctggcacatacgcaggggaagaagcaccagaccaacctggcccggcgagcagccaaggaggccaaggaggcccctgcccagcccgcgcctgagaaggtcaaggtggaggtgaagaagtttgtgaagatcggccgcccgggctacaaagtgaccaagcagagagactcggagatgggccagcagagcctcctcttccagattgactaccctgagatcgccgagggcatcatgccacgtcaccgcttcatgtctgcgtacgagcagaggatcgagcctccggaccggcgctggcagtacctgctcatggccgccgaaccctacgagaccattgccttcaaggtgccgagcagagagatcgacaaggcggagggcaagttctggacacactggaaccgggagaccaagcagttcttcctccagttccactttaagatggagaagcccccggctccacccagcctccctgctggcccccctggggtgaagcggcctccacccccgctgatgaacggtctgccccctcggccaccgctgcctgagtctttgccaccgcccccgccaggaggcctgcctctgccacccatgccccccacagggcctgcgccctcagggcccccgggaccaccccagctacccccgccagctccaggggtccaccccccggccccagtggtgcatccccctgcatctggggtccatcccccagctcctggcgtccaccccccagctcctggcgtccatcccccagcccctggggtccacccaccaacctctggggtccaccccccagctcctggagtccaccctccagcccccggggttcacccaccagcccccggagtccacccaccagcccctggggttcacccaccagccccaggggtccatcctcccccatcagcgggggttcacccccaggccccgggggtgcacccagcagcccccgccgttcaccctcaggccccaggggtgcacccaccagccccagggatgcaccctcaggccccgggggtccacccccaacctcccggggtccatccgtcggctcctggggtccaccctcagcctccgggagttcacccctcaaatcctggggtgcaccccccaactcccatgcccccaatgctgaggcccccacttccctccgaaggcccagggaacatacctccccctcccccaaccaactga

SEQ ID NO: 19 - protein, 464 aa, Homo sapiens
MDFQHRPGGKTGSGGVASSSESNRDRREPLRQLALETIDINKDPYFMKNHLGSYECKLCLTLHNNEGSYLAHTQGKKHQTNLARRAAKEAKEAPAQPAPEKVKVEVKKFVKIGRPGYKVTKQRDSEMGQQSLLFQIDYPEIAEGIMPRHRFMSAYEQRIEPPDRRWQYLLMAAEPYETIAFKVPSREIDKAEGKFWTHWNRETKQFFLQFHFKMEKPPAPPSLPAGPPGVKRPPPPLMNGLPPRPPLPESLPPPPPGGLPLPPMPPTGPAPSGPPGPPQLPPPAPGVHPPAPVVHPPASGVHPPAPGVHPPAPGVHPPAPGVHPPTSGVHPPAPGVHPPAPGVHPPAPGVHPPAPGVHPPAPGVHPPPSAGVHPQAPGVHPAAPAVHPQAPGVHPPAPGMHPQAPGVHPQPPGVHPSAPGVHPQPPGVHPSNPGVHPPTPMPPMLRPPLPSEGPGNIPPPPPTN

SEQ ID NO: 20 - DNA, 2733 bp, Homo sapiens
aagggaagatggagacaatactggagcagcagcggcgctatcatgaggagaaggaacggctcatggacgtcatggctaaagagatgctcaccaagaagtccacgctccgggaccagatcaattctgatcaccgcactcgggccatgcaagataggtatatggaggtcagtgggaacctgagggatttgtatgatgataaggatggattacgaaaggaggagctcaatgccatttcaggacccaatgagtttgctgaattctataatagactcaagcaaataaaggaattccaccggaagcacccaaatgagatctgtgtgccaatgtcagtggaatttgaggaactcctgaaggctcgagagaatccaagtgaagaggcacaaaacttggtggagttcacagatgaggagggatatggtcgttatctcgatctccatgactgttacctcaagtacattaacctgaaggcatctgagaagctggattatatcacatacctgtccatctttgaccaattatttgacattcctaaagaaaggaagaatgcagagtataagagatacctagagatgctgcttgagtaccttcaggattacacagatagagtgaagcctctccaagatcagaatgaactttttgggaagattcaggctgagtttgagaagaaatgggagaatgggacctttcctggatggccgaaagagacaagcagtgccctgacccatgctggagcccatcttgacctctctgcattctcctcctgggaggagttggcttctctgggtttggacagattgaaatctgctctcttagctttaggcttgaaatgtggcgggaccctagaagagcgagcccagagactattcagtaccaaaggaaagtccctggagtcacttgatacctctttgtttgccaaaaatcccaagtcaaagggcaccaagcgagacactgaaaggaacaaagacattgcttttctagaagcccagatctatgaatatgtagagattctcggggaacagcgacatctcactcatgaaaatgtacagcgcaagcaagccaggacaggagaagagcgagaagaagaggaagaagagcagatcagtgagagtgagagtgaagatgaagagaacgagatcatttacaaccccaaaaacctgccacttggctgggatggcaaacctattccctactggctgtataagcttcatggcctaaatatcaactacaactgtgagatttgtggaaactacacctaccgagggcccaaagccttccagcgacactttgctgaatggcgtcatgctcatggcatgaggtgtttgggcatcccaaatactgctcactttgctaatgtgacacagattgaagatgctgtctccttgtgggccaaactgaaattgcagaaggcttcagaacgatggcagcctgacactgaggaagaatatgaagactcaagtgggaatgttgtgaataagaagacatacgaggatctgaaaagacaaggactgctctagtgttgagggatgtagctcagcttttgggctagcccaggcttccctaagatctgctttttctatttctcccaaccaaatcctcttaaagaccctttgctatgtagtctcatggtctagcatgcatcttgtagaaacaaggcatgctggcagattgcagggttgagatgtgttttatctgttttatattttaaaagattctgccagaaaataaaaccagaccttgttctaaagcccagggttatggaccaactcagtgcttcaggtcttaatgcctccatacctcttcctcaccaactttactagtagctgagatttaatgggcacctattatgctacatatcatgttaggtaaatctgacctgacctctttccccaccctcctttgttgctgcttccctgaatgagtattaccccaggatgaggtctgccatcagcttagttagccattgatgcaaatactagggaaagactaggaggatgagccagggttgctactaaggactaagtgtcgcaccaaggtttgccttttgtatttgcataaagaaaggagttggagctgggtgcagtggcttgtgcctgtagtcccagctacttgggaggctgaggcaggagggttgcttgagactagcctaggtaacatagtgagaccctgtctcattaaaaaaaaaaaaaaaaggcatggtggcacgcactgtagtcccagctactcaggagactgaggctagaagatcctttgaacctaggagtttgagaccagcctgggcgatatagtgaggccccatctcaaaaaaaaaaaaaagcggggggggggagttgggctgtgttggaatgggcctgcagcccaacaaacaagggaactaggaccgacagtgacttcaccagcttgctaggtcagaatgagagactggtgggtctgtctacctgtttcttctacaagatccctatttgactgtaaaagtagctaatactcacatgttctccaatcccaggtagccatggtagagttgggtagagttgagcagccgccccaggatccaaatgtggtgtctgaaatggaaagaactaaggcaaccaggaaggcactgatctgccttataagcacagtcatctgaaagtcaggcctgctgcaggacaggatcccccagagaccccatttgcctctcaacactcagaccttcaactgttttttaataaatctactttttaaaaaaaaaaaata

SEQ ID NO: 21 - protein, 501 aa, Homo sapiens
METILEQQRRYHEEKERLMDVMAKEMLTKKSTLRDQINSDHRTRAMQDRYMEVSGNLRDLYDDKDGLRKEELNAISGPNEFAEFYNRLKQIKEFHRKHPNEICVPMSVEFEELLKARENPSEEAQNLVEFTDEEGYGRYLDLHDCYLKYINLKASEKLDYITYLSIFDQLFDIPKERKNAEYKRYLEMLLEYLQDYTDRVKPLQDQNELFGKIQAEFEKKWENGTFPGWPKETSSALTHAGAHLDLSAFSSWEELASLGLDRLKSALLALGLKCGGTLEERAQRLFSTKGKSLESLDTSLFAKNPKSKGTKRDTERNKDIAFLEAQIYEYVEILGEQRHLTHENVQRKQARTGEEREEEEEEQISESESEDEENEIIYNPKNLPLGWDGKPIPYWLYKLHGLNINYNCEICGNYTYRGPKAFQRHFAEWRHAHGMRCLGIPNTAHFANVTQIEDAVSLWAKLKLQKASERWQPDTEEEYEDSSGNVVNKKTYEDLKRQGLL

SEQ ID NO: 22 - DNA, 1275 bp, Homo sapiens
atggctgccgggccgatctccgagcggaatcaggatgccactgtgtacgtggggggcctggatgagaaggttagtgaaccgctgctgtgggaactgtttctccaggctggaccagtagtcaacacccacatgccaaaggatagagtcactggccagcaccaaggctatggctttgtggaattcttgagtgaggaagatgctgactatgccattaagatcatgaacatgatcaaactctatgggaagccaatacgggtgaacaaagcatcagctcacaacaaaaacctggatgtaggggccaacattttcattgggaacctggaccctgagattgatgagaagttgctttatgatactttcagcgcctttggggtcatcttacaaacccccaaaattatgcgggaccctgacacaggcaactccaaaggttatgcctttattaattttgcttcatttgatgcttcggatgcagcaattgaagccatgaatgggcagtacctctgtaaccgtcctatcaccgtatcttatgccttcaagaaggactccaagggtgagcgccatggctcagcagccgaacgacttctggcagctcagaacccgctctcccaggctgatcgccctcatcagctgtttgcagatgcacctcctccaccctctgctcccaatcctgtggtatcatcattggggtctgggcttcctccaccaggcatgcctcctcctggctccttcccacccccagtgccacctcctggagccctcccacctgggatacccccagccatgcccccaccacctatgcctcctggggctgcaggacatggccccccatcggcaggaaccccaggggcaggacatcctggtcatggacactcacatcctcacccattcccaccgggtgggatgccccatccagggatgtctcagatgcagcttgcacaccatggccctcatggcttaggacatccccacgctggacccccaggctctgggggccagccaccgccccgaccaccacctggaatgcctcatcctggacctcctccaatgggcatgcccccccgagggcctccattcggatctcccatgggtcacccaggtcctatgcctccgcatggtatgcgtggacctcctccactgatgcccccccatggatacactggccctccacgacccccaccctatggctaccagcgggggcctctccctccacccagacccactccccggccaccagttccccctcgaggcccacttcgaggccctctccctcagtaa

SEQ ID NO: 23 - protein, 424 aa, Homo sapiens
MAAGPISERNQDATVYVGGLDEKVSEPLLWELFLQAGPVVNTHMPKDRVTGQHQGYGFVEFLSEEDADYAIKIMNMIKLYGKPIRVNKASAHNKNLDVGANIFIGNLDPEIDEKLLYDTFSAFGVILQTPKIMRDPDTGNSKGYAFINFASFDASDAAIEAMNGQYLCNRPITVSYAFKKDSKGERHGSAAERLLAAQNPLSQADRPHQLFADAPPPPSAPNPVVSSLGSGLPPPGMPPPGSFPPPVPPPGALPPGIPPAMPPPPMPPGAAGHGPPSAGTPGAGHPGHGHSHPHPFPPGGMPHPGMSQMQLAHHGPHGLGHPHAGPPGSGGQPPPRPPPGMPHPGPPPMGMPPRGPPFGSPMGHPGPMPPHGMRGPPPLMPPHGYTGPPRPPPYGYQRGPLPPPRPTPRPPVPPRGPLRGPLPQ

SEQ ID NO: 24 - DNA, 1054 bp, Homo sapiens
gaattccgcgggaggccacgggctttccacagcgcgggggaacgggaggctgcaggatggtcaagctgacggcggagctgatcgagcaggcggcgcagtacaccaacgcggtgcgcgaccgggagctggacctccgggggtataaaattcccgtcattgaaaatctaggtgctacgttagaccagtttgatgctattgatttttctgacaatgagatcaggaaactggatggttttcctttgttgagaagactgaaaacattgttagtgaacaacaacagaatatgccgtataggtgagggacttgatcaggctctgccctgtctgacagaactcattctcaccaataatagtctcgtggaactgggtgatctggaccctctggcatctctcaaatcgctgacttacctaagtatcctaagaaatccggtaaccaataagaagcattacagattgtatgtgatttataaagttccgcaagtcagagtactggatttccagaaagtgaaactaaaagagcgtcaggaagcagagaaaatgttcaagggcaaacggggtgcacagcttgcaaaggatattgccaggagaagcaaaacttttaatccaggtgctggtttgccaactgacaaaaagagaggtgggccatctccaggggatgtagaagcaatcaagaatgccatagcaaatgcttcaactctggctgaagtggagaggctgaaggggttgctgcagtctggtcagatccctggcagagaacgcagatcagggcccactgatgatggtgaagaagagatggaagaagacacagtcacaaacgggtcctgagcagtgaggcagatgtataataataggccctcttggaacaagtcttgcttttcgaacatggtataatagccttgtttgtgttagcaaagtggaatctatcagcattgttgaaatgcttaagactgctgctgataattttgtaatataagttttgaaatctaaatgtcaattttctacaaattataaaaataaactccactctctatgctaaaaaaaaaaaaaaggaattc

SEQ ID NO: 25 - protein, 255 aa, Homo sapiens
MVKLTAELIEQAAQYTNAVRDRELDLRGYKIPVIENLGATLDQFDAIDFSDNEIRKLDGFPLLRRLKTLLVNNNRICRIGEGLDQALPCLTELILTNNSLVELGDLDPLASLKSLTYLSILRNPVTNKKHYRLYVIYKVPQVRVLDFQKVKLKERQEAEKMFKGKRGAQLAKDIARRSKTFNPGAGLPTDKKRGGPSPGDVEAIKNAIANASTLAEVERLKGLLQSGQIPGRERRSGPTDDGEEEMEEDTVTNGS

SEQ ID NO: 26 - DNA, 378 bp, Homo sapiens
atggcgatgcaagcggccaagagggcgaacattcgacttccacctgaagtaaatcggatattgtatataagaaatttgccatacaaaatcacagctgaagaaatgtatgatatatttgggaaatatggacctattcgtcaaatcagagtggggaacacacctgaaactagaggaacagcttatgtggtctatgaggacatctttgatgccaagaatgcatgtgatcacctatcgggattcaatgtttgtaacagataccttgtggttttgtactataatgccaacagggcatttcagaagatggacacaaagaagaaggaggaacagttgaagcttctcaaggagaaatatggcatcaacacagatcctcccaagtga

SEQ ID NO: 27 - protein, 125 aa, Homo sapiens
MAMQAAKRANIRLPPEVNRILYIRNLPYKITAEEMYDIFGKYGPIRQIRVGNTPETRGTAYVVYEDIFDAKNACDHLSGFNVCNRYLVVLYYNANRAFQKMDTKKKEEQLKLLKEKYGINTDPPK

SEQ ID NO: 28 - DNA, 904 bp, Homo sapiens
ggaattccgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttaaaaaacaacaaaaaaaaaacatacaaagatgggtttctgaataaaaatttgtagtgataacagt

SEQ ID NO: 29 - protein, 240 aa, Homo sapiens
MAEYLASIFGTEKDKVNCSFYFKIGACRHGDRCSRLHNKPTFSQTIALLNIYRNPQNSSQSADGLRCAVSDVEMQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 30 - DNA, 1707 bp, Homo sapiens
gaggcgaaagctgcacagggccctacgcggccgcctcagcatgtcggacttcgacgagttcgagcggcagctcaacgagaataaacaagagcgggacaaggagaaccggcatcggaagcgcagccacagccgctctcggagccgggaccgcaaacgccggagccggagccgcgaccggcgcaaccgggaccagcggagcgcctcccgggacaggcgacgacgcagcaaacctttgaccagaggcgctaaagaggagcacggtggactgattcgttccccccgccacgagaagaagaagaaggtccgtaaatactgggacgtgccacccccaggctttgagcacatcaccccaatgcagtacaaggccatgcaagctgcgggtcagattccagccactgctcttctccccaccatgacccctgacggtctggctgtgaccccaacgccggtgcccgtggtcgggagccagatgaccagacaagcccggcgcctctacgtgggcaacatcccctttggcatcactgaggaggccatgatggatttcttcaacgcccagatgcgcctgggggggctgacccaggcccctggcaacccagtgttggctgtgcagattaaccaggacaagaattttgcctttttggagttccgctcagtggacgagactacccaggctatggcctttgatggcatcatcttccagggccagtcactaaagatccgcaggcctcacgactaccagccgcttcctggcatgtcagagaacccctccgtctatgtgcctggggttgtgtccactgtggtccccgactctgcccacaagctgttcatcgggggcttacccaactacctgaacgatgaccaggtcaaagagctgctgacatcctttgggcccctcaaggccttcaacctggtcaaggacagtgccacggggctctccaagggctacgccttctgtgagtacgtggacatcaacgtcacggatcaggccattgcggggctgaacggcatgcagctgggggataagaagctgctggtccagagggcgagtgtgggagccaagaatgccacgctggtgagccccccgagcaccatcaatcagacgcctgtgaccctgcaagtgccgggcttgatgagctcccaggtgcagatgggcggccacccgactgaggtcctgtgcctcatgaacatggtgctgcctgaggagctgctggacgacgaggagtatgaggagatcgtggaggatgtgcgggacgagtgcagcaagtacgggcttgtcaagtccatcgagatcccccggcctgtggacggcgtcgaggtgcccggctgcggaaagatctttgtggagttcacctctgtgtttgactgccagaaagccatgcagggcctgacgggccgcaagttcgccaacagagtggttgtcacaaaatactgtgaccccgactcttatcaccgccgggacttctggtagaggcggctgggggagggtgggggcagggctggctgggggcttctccccactcccgcccccccccttatccccctctgaagacgatgggcagaggagtgacagccgcagacacacgacagccggcagcaactggaatggcagcaattaagggtggggagcgggggttggggggttggggggttagggcagggaggggactggggaagtgcgcacacagccccacacagacaacacgcacg

SEQ ID NO: 31 - protein, 475 aa, Homo sapiens
MSDFDEFERQLNENKQERDKENRHRKRSHSRSRSRDRKRRSRSRDRRNRDQRSASRDRRRRSKPLTRGAKEEHGGLIRSPRHEKKKKVRKYWDVPPPGFEHITPMQYKAMQAAGQIPATALLPTMTPDGLAVTPTPVPVVGSQMTRQARRLYVGNIPFGITEEAMMDFFNAQMRLGGLTQAPGNPVLAVQINQDKNFAFLEFRSVDETTQAMAFDGIIFQGQSLKIRRPHDYQPLPGMSENPSVYVPGVVSTVVPDSAHKLFIGGLPNYLNDDQVKELLTSFGPLKAFNLVKDSATGLSKGYAFCEYVDINVTDQAIAGLNGMQLGDKKLLVQRASVGAKNATLVSPPSTINQTPVTLQVPGLMSSQVQMGGHPTEVLCLMNMVLPEELLDDEEYEEIVEDVRDECSKYGLVKSIEIPRPVDGVEVPGCGKIFVEFTSVFDCQKAMQGLTGRKFANRVVVTKYCDPDSYHRRDFW

SEQ ID NO: 32 - DNA, 1488 bp, Homo sapiens
gaggggcggggcggtgccggcaagatggctgcgcccgagaagatgacgtttcccgagaaaccaagccacaaaaagtacagggccgccctgaagaaggagaaacgaaagaaacgtcggcaggaacttgctcgactgagagactcaggactctcacagaaggaggaagaggaggacacttttattgaagaacaacaactagaagaagagaagctattggaaagagagaggcaaagattacatgaggagtggttgctaagagagcagaaggcacaagaagaattcagaataaagaaggaaaaggaagaggcggctaaaaaacggcaagaagaacaagagagaaagttaaaggaacaatgggaagaacagcagaggaaagagagagaagaggaggagcagaaacgacaggagaagaaagaaaaagaggaagctttgcagaagatgctggatcaggctgaaaatgagttggaaaatggtaccacatggcaaaacccagaaccacccgtggatttcagagtaatggagaaggatcgagctaattgtcccttctacagtaaaacaggagcttgcagatttggagatagatgttcacgtaaacataatttcccaacatccagtcctacccttcttattaagagcatgtttacgacgtttggaatggagcagtgcaggagggatgactatgaccctgacgcaagcctggagtacagcgaggaagaaacctaccaacagttcctagacttctatgaggatgtgttgcccgagttcaagaacgtggggaaagtgattcagttcaaggtcagctgcaatttggaacctcacctgaggggcaatgtatatgttcagtaccagtcggaagaagaatgccaagcagccctttctctgtttaacggacgatggtatgcaggacgacagctgcagtgtgaattctgccccgtgacccggtggaaaatggcgatttgtggtttatttgaaatacaacaatgtccaagaggaaagcactgcaactttcttcatgtgttcagaaatcccaacaatgaattctgggaagctaatagagacatctacttgtctccagatcggactggctcctcctttgggaagaactccgaaaggagggagaggatgggccaccacgacgactactacagcaggctgcggggaaggagaaaccctagtccagaccactcctacaaaagaaatggggaatccgagaggaaaagtagtcgtcacagggggaagaaatctcacaaacgcacatcaaagagtcgggagaggcacaattcacgaagcagaggaagaaatagggaccgcagcagggaccgcagccggggccggggcagccggagccggagccggagccggagccgcaggagccgccgcagccggagccaaagttcctctaggtcccgaagtcgtggcaggaggaggtcgggtaatagagacagaactgttcagagtcccaaatccaaataaactagttttgttctt

SEQ ID NO: 33 - protein, 482 aa, Homo sapiens
MAAPEKMTFPEKPSHKKYRAALKKEKRKKRRQELARLRDSGLSQKEEEEDTFIEEQQLEEEKLLERERQRLHEEWLLREQKAQEEFRIKKEKEEAAKKRQEEQERKLKEQWEEQQRKEREEEEQKRQEKKEKEEALQKMLDQAENELENGTTWQNPEPPVDFRVMEKDRANCPFYSKTGACRFGDRCSRKHNFPTSSPTLLIKSMFTTFGMEQCRRDDYDPDASLEYSEEETYQQFLDFYEDVLPEFKNVGKVIQFKVSCNLEPHLRGNVYVQYQSEEECQAALSLFNGRWYAGRQLQCEFCPVTRWKMAICGLFEIQQCPRGKHCNFLHVFRNPNNEFWEANRDIYLSPDRTGSSFGKNSERRERMGHHDDYYSRLRGRRNPSPDHSYKRNGESERKSSRHRGKKSHKRTSKSRERHNSRSRGRNRDRSRDRSRGRGSRSRSRSRSRRSRRSRSQSSSRSRSRGRRRSGNRDRTVQSPKSK

SEQ ID NO: 34 - DNA, 5695 bp, Homo sapiens
agcacgtcctgttttcgttggccgcgctgggatggccgccacagctgtaggtgctgctagtgtttagcgctggtctttgccgggcgttgagggcagctcagcctccttgtttgtccggttcgcctgtgcgtggtactcaagggcaccagtattcccgcggtcggcagcatgggtcgggagtcacgccactatcgaaaacgatcggcatcccggggtcgctctggaagtcggtctagaagtcgctcaccctcagacaaaagaagtaaacgtggagatgacagacggtctagaagtagagatagagataggaggagagagaggtctcgtagcagggataaaagaagatctcggtcaagggacaggaagcgtctgagacgttccagaagtagagagagagacagaagccgagagcgaagaagatctcgaagtagagacaggagacgctcaaggagtagaagccggggccggcgatcccgatcctccagtcctggaaataaaagcaagaaaactgagaatagatctaggtccaaagagaaaactgatggtggggaaagttctaaagagaagaaaaaagacaaagatgacaaggaggatgaaaaagaaaaagatgctggcaactttgaccagaataagctggaagaagaaatgagaaagcgaaaagaaagagtagaaaaatggcgagaagagcaacgtaaaaaggctatggaaaacataggagaactgaaaaaggaaatcgaagagatgaaacaagggaaaaagtggagtttagaggacgatgatgatgacgaagatgatcctgcagaagctgaaaaggagggaaatgaaatggagggtgaggagttagatccattagatgcttacatggaagaagtgaaagaggaagtaaaaaaatttaacatgagaagtgtaaaaggtggtgggggaaatgaaaagaagtctgggccaacggtcacaaaagttgtcactgttgtgacaaccaaaaaagcagttgtggattctgataagaagaaaggtgagctgatggagaatgaccaggatgccatggagtattcttcagaggaggaagaagttgatcttcagacagcccttacagggtatcaaacaaaacagcgaaagcttctagaaccagttgatcatggaaaaattgagtatgagccatttaggaaaaacttctatgttgaagttccagaactagcaaaaatgtctcaagaagaggtaaatgtgtttcgattggaaatggagggcattacagttaaaggaaaaggttgccccaaaccaattaaatcctgggtccagtgtggaatttccatgaagatcttaaattccctcaagaagcatggctatgaaaagcccacgcccatccaaacccaagctattcctgctataatgtctggacgagatttgattggcattgccaaaacaggaagtggaaagaccattgcttttctgttgcccatgtttagacacatcatggatcagaggtcattagaggaaggagaggggccaatagctgtcatcatgactccaactcgagaactggctttacagattactaaagagtgtaagaagttttccaagactttgggacttagagtggtctgtgtttacggaggaacaggaatcagtgagcagattgctgagctgaaaagaggtgctgaaattattgtttgcacacctggtcgaatgattgacatgttagccgctaacagtggtcgggtcacaaatcttcgaagagtgacatatgttgttttagatgaagcagacagaatgtttgacatgggttttgaaccccaggtcatgcgcatcgtggataatgttcgtcctgatcgacagacggttatgttttcagctactttccccagagctatggaggctttggctcgcaggatcctcagtaaacctattgaagtacaagttggaggcaggagtgtggtttgctcagatgtggagcaacaagtgattgtgattgaagaagaaaagaaattcttgaagttacttgagcttctaggccattatcaagagtcaggatctgtcattatatttgtggataagcaggaacatgctgatggtcttcttaaggatttaatgagagcatcttatccttgcatgtctcttcatggaggcattgatcaatatgacagagatagcatcataaatgactttaagaatgggacctgcaaacttcttgtggctacctctgttgctgcccgaggtctagatgtgaaacatctgattcttgtagtaaattatagctgccccaaccattatgaggattatgtacacagagcagggcggactggaagagcaggaaacaagggttatgcttatacttttatcacagaggatcaagctcgctatgctggtgacataattaaagctcttgaattgtcagggactgcagtacctcctgatttagagaaactgtggagtgatttcaaagatcagcagaaagctgaggggaaaataattaaaaagagtagtgggttctctggtaagggattcaagtttgatgaaacagaacaagctttggctaatgagaggaagaagttacaaaaagcagctcttggtctacaagattcagatgatgaggatgctgcagttgatattgatgagcaaattgaaagcatgtttaattcaaagaagagagtaaaggatatggctgctcctggaacatcaagtgttcctgctccaactgcaggaaatgctgagaaattagaaattgctaagagattggctcttagaatcaatgcccagaagaatttgggcatcgagtctcaggatgtgatgcagcaggccaccaatgcaattcttaggggtggcaccattctggctcccactgtttctgcaaaaaccattgcagaacaacttgctgaaaagatcaatgccaagctcaattatgtgccgttagagaaacaagaagaagagagacaggatggtggacagaatgaatcttttaagagatatgaagaagaattagagatcaatgacttcccacagactgctaggtggaaagttacctctaaggaagctctgcagagaatcagtgaatactctgaagccgcaattacaatcagaggaacctacttccctcctggcaaagaacccaaggaaggcgagcggaagatttacttggcaattgaaagtgccaatgaactggctgtgcagaaagcaaaggcagaaatcaccaggctcataaaagaagagctgatccggctgcaaaattcataccaaccaacaaataaaggaagatacaaagtcttatagacatccggaaaaaagatttttacctgtgctggtctatgatgtatgtggcagttgctgtctgcagtttacaatgtattgtaaatgaagattttttaaattctatcttgctgattttttttaaatataagaaactggtacttggtaaagaaatctgtccgtaagtacccccacaatcagtcaaactatatttaaagccagcctgttttcagagtatgatgtcctttaatgtaaactcaaatatcaatattttaaatgtccggataatattctagaggtttaaaaaatggaaatatttgaactttctattgaagacaataaagtacacaagtcgttaaggggctattcactttatcctgtactttcaatgaaattgtgatcatttcctaagaaaaggtaaaattcactatcatattttgtgtccccaccttgatgttacatgactctggaacaatatgaactggatttaagaatgttataatagaagtcttacaaaatgggttgagagtttttgtttcaatttttatcatcataaatgggcaaaaagtagttggactaatttcggtttttatacaagtaaagatttaatagtataaggatttttttgcatttctttacactgagtgtaaaactctacaaagagttatagtatttactactttgaggtttccctcacaacttctggctccatacctagcccctcttttataatcttccttaaaagaaagagtgtagcctataaatactaaatatgataccttttccttctagaaagtgtttatttatatatctatacatgttgtatgtacaaatatcctactacttttaatctgatttttcttcaggattattgagtaggttgtgaattttctttcttaaaaattgtaaaacataatggtacccaagttttaaacttagatgtgcttcatcttagtgaaatttaattcacaaggaatcataaattgtgtttttgaggctgggcgcagtgactcacacctgtatcccagcaatttgggaggccgaggtgggcagatcacttgaggtcaggagttcaagactagcctggtcaacatggtgaaaccctgtctctactaaaaatacaaaaattagtcggcatggtggtgggtggctgtaatcccagctactcaggaggctgaggcagaagaatctgagccaagataacaccattgcactccagcctgggcgacagagtgagactatctcaaaaataaataaataaataaataaatagtatttttgagccttcaagatacagttcctgaatattctaatttaaattgttataattggattacctaatattcttttcaagactcctgatgtattaacttcatgttattgtcacttttcaaaaaaggaatttttttcaggagttaaataaagtcagtcttttatatccaagggtttcaaacccacagattaaaccaaccacagatcaaaaatagaaaaaaaaaaaaaaagaaaaaaagaatgtaaaattttaaaaatacagtttaacagctgtaaaagttttactataacatttacattgtgttaggtattttaagtaatttaaaggttacagtatacaggcggttaaagtatacaggaggatgtgcatcggttatatgcatatagcaagccatttcatataagaggcttgaacatccatggattttggtatagaggaggggtttaaggggtgtcctgcaatcagtccccctctcataccaaaggatgactgtatatttcttaaatgaagagactttcattctttaaatattattgacttacagtttaaaaataaataaataaaagcatactatagttgtaaaaacaaataattttaatagtatattcagtggctgaagtgatagggagtattaatcattatttcttcagtataaagttattcttcctgaaaaagtaagatatacctgggaaatattcttggatccttcacacatccacttaatttcttgaaggaagaaaacagacaaaagacatgtaacaacagaaacaaataaaccacttactgtaaaatttcagaaattgtgggctcattccctccctctagtttatttatttgctcatctttatcacctaatggtagtttgttttctttggtagagtatatggcagtcccacatcgatgataattggtgctcccttcatccaactggatcactttgagattcagttttacatcattttagcattttgtttgtatatcagttgtgatgagccactgataaaattttgttctaaaatgaaacatattactttgttagtacattttttagtgtcacactgacttttaaaatttgaatgatatatagactttgtttttttaatgcaatatgaatatttgctcatgttttctaaattccttataaaataaaattaattcgaattcaaaaaaaaaaaaaaaaaa

SEQ ID NO: 35 - protein, 1031 aa, Homo sapiens
MGRESRHYRKRSASRGRSGSRSRSRSPSDKRSKRGDDRRSRSRDRDRRRERSRSRDKRRSRSRDRKRLRRSRSRERDRSRERRRSRSRDRRRSRSRSRGRRSRSSSPGNKSKKTENRSRSKEKTDGGESSKEKKKDKDDKEDEKEKDAGNFDQNKLEEEMRKRKERVEKWREEQRKKAMENIGELKKEIEEMKQGKKWSLEDDDDDEDDPAEAEKEGNEMEGEELDPLDAYMEEVKEEVKKFNMRSVKGGGGNEKKSGPTVTKVVTVVTTKKAVVDSDKKKGELMENDQDAMEYSSEEEEVDLQTALTGYQTKQRKLLEPVDHGKIEYEPFRKNFYVEVPELAKMSQEEVNVFRLEMEGITVKGKGCPKPIKSWVQCGISMKILNSLKKHGYEKPTPIQTQAIPAIMSGRDLIGIAKTGSGKTIAFLLPMFRHIMDQRSLEEGEGPIAVIMTPTRELALQITKECKKFSKTLGLRVVCVYGGTGISEQIAELKRGAEIIVCTPGRMIDMLAANSGRVTNLRRVTYVVLDEADRMFDMGFEPQVMRIVDNVRPDRQTVMFSATFPRAMEALARRILSKPIEVQVGGRSVVCSDVEQQVIVIEEEKKFLKLLELLGHYQESGSVIIFVDKQEHADGLLKDLMRASYPCMSLHGGIDQYDRDSIINDFKNGTCKLLVATSVAARGLDVKHLILVVNYSCPNHYEDYVHRAGRTGRAGNKGYAYTFITEDQARYAGDIIKALELSGTAVPPDLEKLWSDFKDQQKAEGKIIKKSSGFSGKGFKFDETEQALANERKKLQKAALGLQDSDDEDAAVDIDEQIESMFNSKKRVKDMAAPGTSSVPAPTAGNAEKLEIAKRLALRINAQKNLGIESQDVMQQATNAILRGGTILAPTVSAKTIAEQLAEKINAKLNYVPLEKQEEERQDGGQNESFKRYEEELEINDFPQTARWKVTSKEALQRISEYSEAAITIRGTYFPPGKEPKEGERKIYLAIESANELAVQKAKAEITRLIKEELIRLQNSYQPTNKGRYKVL

SEQ ID NO: 36 - DNA, 2221 bp, Homo sapiens
atttccgttaggtgctgaaggctgtggcgcgcggctgtccccattcccacgtgaagcgctacgctagcatcgctcggctggcggctcccagctcgccgcggagcagtcccggcagcagcgggggaccggaagtggctcgcggaggctcagaagctagtcccggagcccggcgtgtggcgcctcggagcacggtgacggcgccatgtccctaatctgctccatctctaacgaagtgccggagcacccatgtgtatcccctgtctctaatcatgtttatgagcggcggctcatcgagaagtacattgcggagaatggtaccgaccccatcaacaaccagcctctctccgaggagcagctcatcgacatcaaagttgctcacccaatccggcccaagcctccctcagccaccagcatcccggccattctgaaagctttgcaggatgagtgggatgcagtcatgctgcacagcttcactctgcgccagcagctgcagacaacccgccaagagctgtcacacgctctgtaccagcacgatgccgcctgccgtgtcattgcccgtctcaccaaggaagtcactgctgcccgagaagctctggctaccctgaaaccacaggctggcctcattgtgccccaggctgtgccaagttcccaaccaagtgttgtgggtgcgggtgagccaatggatttgggtgagctggtgggaatgaccccagagattattcagaagcttcaagacaaagccactgtgctaaccacggagcgcaagaagagagggaagactgtgcctgaggagctggtgaagccagaagagctcagcaaataccggcaggtggcatcccacgtggggttgcacagtgccagcattcctgggatcctggccctggacctctgcccgtccgacaccaacaagatcctcactggtggggcggataaaaatgtcgttgtgtttgacaaaagttctgaacaaatcctggctaccctcaaaggccataccaagaaggtcaccagcgtggtgtttcacccttcccaggacctggtgttttctgcttcccccgatgccactatcaggatttggtcggtccccaatgcctcttgtgtacaggtggttcgggcccatgagagtgctgtgacaggcctcagccttcatgccactggcgactatctcctgagctcctccgatgatcagtactgggctttctctgacatccagacagggcgtgtgctcaccaaggtgacagatgagacctccggctgctctctcacctgtgcacagttccaccctgacggactcatctttggaacaggaaccatggactctcagatcaagatctgggacttgaaggaacgtactaatgtggccaacttccctggccactcgggccccatcactagcatcgccttctctgagaatggttactacctggctacagcggctgatgactcctctgtcaagctctgggatctgcgcaagcttaagaactttaagactttgcagctggataacaactttgaggtaaagtcactgatctttgaccagagtggtacctacctggctcttgggggcacggatgtccagatctacatctgcaaacaatggacggagattcttcactttacagagcatagcggcctgaccacaggggtggccttcgggcatcacgccaagttcatcgcttcaacaggcatggacagaagcctcaagttctacagcctgtaggccctggcccttctgatggaagctgggcctcatctcagtagaggggtagaattagggtttggggggggggtggggggaatctatggggggagggggctctgtggggtgggacattcacatcatttcactctggtctgagtggtggcctgagaaccatggtggcatggaccaccctcatccatgcaactccaggccccatgggaacggatgtggaaggaagaactgtcaccctcttaaggcccagggtcggagcccagggcctctcccttcctgtcgttcaatggacgtggtggtggctgttccacacccattttgttgcagttcctgtgagacaggagaggctgagccaagggaactgtgaaggggatgggcaggagggcttgtgcagggttttgtaagcagtgatctagtttcattaaaaaaagaaaacaataaaaaaaaaaaaaaaaaaaaaaaaaaaaaaaaaaaaaaaaaaaaaaaaaaaaaaaaaaaaaaaaaaaaaaa

SEQ ID NO: 37 - protein, 504 aa, Homo sapiens
MSLICSISNEVPEHPCVSPVSNHVYERRLIEKYIAENGTDPINNQPLSEEQLIDIKVAHPIRPKPPSATSIPAILKALQDEWDAVMLHSFTLRQQLQTTRQELSHALYQHDAACRVIARLTKEVTAAREALATLKPQAGLIVPQAVPSSQPSVVGAGEPMDLGELVGMTPEIIQKLQDKATVLTTERKKRGKTVPEELVKPEELSKYRQVASHVGLHSASIPGILALDLCPSDTNKILTGGADKNVVVFDKSSEQILATLKGHTKKVTSVVFHPSQDLVFSASPDATIRIWSVPNASCVQVVRAHESAVTGLSLHATGDYLLSSSDDQYWAFSDIQTGRVLTKVTDETSGCSLTCAQFHPDGLIFGTGTMDSQIKIWDLKERTNVANFPGHSGPITSIAFSENGYYLATAADDSSVKLWDLRKLKNFKTLQLDNNFEVKSLIFDQSGTYLALGGTDVQIYICKQWTEILHFTEHSGLTTGVAFGHHAKFIASTGMDRSLKFYSL

SEQ ID NO: 38 - DNA, 6075 bp, Homo sapiens
ggtcgtgcgcgctgaggaggagccgctgccgccgtcgccgtcgccgccaccgccgccaccgctaccgaggccgagcggagccgttagcgccgcgccgccgccgcctcccgcccgccccggagcagccccgggcccgcccgcccgcatccagatttttgaaaaatacaatgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataactcgctcatgcttttttttgtacggaatagataattaagagtgaaggagttgaaacttttcttgttagtgtacaactcattttgcgccaattttcacaagtgtttgtctttgtctgaatgagaagtgagaaggtttttatactctgggatgcaaccgacatgttcaaatgtttgaaatcccacaatgttagaccaatcttaagtttcgtaagttatttcctttaagatatatattaaacagaaatctaagtagaactgcattgactaaccagtccctctggatggtggtgaacctgaagcatgctttaacctctaagactgtctaacacgcgtttcattcaatgtctccacagactgggtagcaaaaaaatcaccttttagttttagtttttaatctaaagatgttagacagatgctgagtgtgcgttttctcaaccgcttcaacattgtaagcgatgtatgctttggttgacaggaagttccttttccaggcaggtcccgttgccacctcctgctcactcagtcccgggctctgccgagtggtcctgggaatggcggcgggcccgtccagcgtgggccaccactggggccgggggccacgggctgcatgctgggcgggccctccagagaaggacacaaacgtgtttcgtaagcccaggcaccaatgggaatggaccaaagagtttcagggaaactccagtatattccagagtcagatctaagctccaggcacgcctgaagatgtgttgctactctgacatcccgagtttctgtccacacattgcatgcacagcgccccacacattggatactgttgttcacgataatttctcccgttttccagagcatttaacatagcttggaggcgtaaaatggctctgtattttaataacacagaaacatttgagcattgtatttctcgcatcccttctcgtgagcgcttagacctttttctattttagtcggattttgttttggaattttgcttttgtatgaacactcagcagaaaagtacttacttcttgccagttatctattaaccaaaacctttgatttgtagttttaaagattaaccctcaaagttctcttcataactgccttgacattttgggttgttctgttctgttaattttcttttgcttttttgtgttttttgtttgtttttacttttgcatttaagaccattaaatttgattttgttttgctcgaattttgttttgttttttattttaccttttctttctttttggctagggaaggtgcaggtggcccagcattcagggaggagtcgtaagatcttaagaaaccaatccttgcctcaagcaaaagcatttctgaatctgtgacgcaagaatgtgcagttacaggctggtggcttttaaaccaggagcccggaggaagggtgaaagagaaagcctgtgaaataggcagggccagatcacccaaaactcctcaggactgggatctggcgtttataaataactagtttacagagagaatcacaaacaggataacttagtaccagcagtttttaaccttgacgtgagactaaaacgtgaccgtaggctgttttttagttattgctctcatgagatgatggctgtatttatctgtttatttatacctatttatgtatttatttattgaagtgtgaaattgagcaataggcaggcaccaccgtccccagagcaggtcagcgtctcgagaggcccctggacaattgaggatgcccatcccctcccctttccctgatcttttactgaggggctgtgtgcgcgatccttgcaaattgatgatgttgccatccgtacccaggctgtgtctcataaaagtcggcctggtgccagagaggacctcctttctcccacagaatcccagatcctcaggaaaagccaaaaccgaggcccattgcccggattcgacacaaaagagggtccctgctctgttgcccgagagcagtctgcatcctgggaccagaatgctttcctggaaaaagaagcctttcaggtttccctgggccagcatcttctgatggaaggtgggagccaacacccttctgatggaaggtgggagccaacacccttctgatggaaggtgggagccaacacccttctgatggaaggtgggagccaacacccttctgatggaaggtgggagccaacacccttctgatggaaggcgggattcccgcttctgaaactccccctggagtctcactcccacacatgcccatagctagcattcaacagagaactctgtcttaagcttcaactgtgaaaatgatgacgggcttgtagcacctcagcttctttcctcgcccccttttatctgaatcctatcaattattctgatgctgggacaggtgagaagaaactgtgaagtatatgagcctttgaaagttccctgaagtttctcagttcaggaacattctcattgtatgtggtctccgctgtttgaacagccttctagctaaaaaattccaaagcctttatttgggagtcttagcttgcaagcttgtggaaggatttagcttaacaactgtcactcctgaaaagcaatctctgttccatcaaggttctagttgctggccctgtgtcctcaaagttcattacatcttatcaaggcctgtttgcaaaggggagatcccttttcttaaaaaaggctcaacccaaaagaaaccatttcttaaaaaattttacatagatcagttgtatttctatttagcaaaaatgagtgctctgcttttatttgggaatttcgatgaaaaagcgttcagagtagataatgttcatttatcaaaaatctggtttgggaaataccaaagaggctttgattgaattcccttttgacccgtgtgtaacttcctctggtagttagaccccaggcagctccgaatttgtgaacctgcttcctgatgaattctcccttgttcccccttggctctgccattatttcgttttcagtgtaatttgccaagccgcagttttctgtgctggctgtgcctctagtcgcagctctgtgactgattccctcccgggtgctgagtcccctccccggccaccatcctgcgtgaatatcctgaaattcatgggcttcctcgggggcccgcccgcaggtggtgctgggtgggttccgccaccttctcctggaaggtgagccttttcctggccaagggcagctgccttaacctctgagagtctgcgcttggccttagtcctggagacccagcctccagggactgaaccgtgctgctgttgggagccaagaccggccctttggagccggcagcccaggggtccctgctggatcagagaaatagaagcacccgaagacggttagtggcaattccttgacccggtttgcttccaaatgaaggccatttgtccaccaggcattgaaaagacatgacttacccagtccggcatcggacttgaaaaatcgaaattgacatcactcagctgttacatttcacatccgattcagcccccttttatttccatgtgcttttcgcagccttcctgtgttggatgaaagagaataagaattcagctgacaggaggcctctatcctgtccctccaccccaccctccacctcaatcccctcccatcttccccagacctacctcacctactaggacctgaggcagctccttagcagagacccctggggtttagctgactctgggggtcaggggttcctgtccccaaacttcgcaagacagccctgaagtcacaagtgctttcttttaagtggcattggcaattggcgtgtaatgatggcagtagaatctgaatctcggatcccaggcagggttcacatttccaaacctttttgatttcccctgacctctaatggctggatcctatttttctacaacctttcagtgacatcgttcaggtttctttcttggccatttaaaaaaacaaattttttttttctcacttgtaagtcaccgccagtacctaagttaggctaacggagactttgacaggactggatttttcttccaccagaagagaagccttttccgttggtttggggccacctctttgaccatgaccatgtgatgttccgtttacagtgacttgctttgggggaggggaggctctcttaaccgattcccatgttgtacagtagatggttagaccttttgtatattagtgtgttttaagttattgatttgttttatataaaataatttatttttcaggtgccatttttcattttaactttgtttttacatgggtttgttttcaataaagtctgacactggtgtccaaaagtcaacaataaaatgaatcccattgtgttcttttgaagatgcctatgtaacttttaagctttttaaattattttcagaaaaaaaaaaagaaaagcccttatcagttttccatcagcccattgcctttttattttttttttttaatccttgtgaataaatgttctttagtgttttaggaggaaaaagcaaacctagattttgataacccagaagacttcagattaataaagaagctttgaaagaagaccatttttcaaaattttagtgaagtgtgaatattttttgtcaatggctttctcaaagagaatgaaacttttgcaccattttcagagtttttatagagatgccaaattgatatatttacatgtaatggaaacatgaaaaagttttattaaacaattgttcatagctgtgtagacattttaattcagtttccaaagctctcaaaaaatcgtatttttgaagtacggagtgatgcggtttggggcgtggcttacagttccaacgactcaattgtcccgatactcagttctttctacaggtatcaggttcgtgttaaacgctgtatgttaactatgactggaattctgtgatattttggtaataaatgaagtgggatcattgcgaaaaaaaaaaaaaaaaaa

SEQ ID NO: 39 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 40 - DNA, 1105 bp, Homo sapiens
gagccgatgcccgattccgcgcccgccatggccgacaaaatggacatgtctctggacgacatcattaaactgaaccggagccagcgaggcggccggggcgggggccggggccgcggccgggccggctcccagggcggccgcggcggtggggcgcaggccgccgcgcgagtgaatcgaggcggcgggcccatccggaaccggccggccatcgcccgcggcgcggccggcggaggcggcaggaaccgaccggcgccctacagcaggccaaaacaacttcccgacaagtggcagcacgatcttttcgacagtggcttcggcggtggtgccggcgtggagacaggtgggaaactgctggtgtccaatctggattttggagtctcagacgccgatattcaggaactctttgctgaatttggaacgctgaagaaggcggctgtgcactatgatcgctctggtcgcagcttaggaacagcagacgtgcactttgagcggaaggcagatgccctgaaggccatgaagcagtacaacggcgtccctctggatggccgccccatgaacattcagcttgtcacgtcacagattgacgcacagcggaggcctgcacagagcgtaaacagaggtggcatgactagaaaccgtggcgctggaggttttggtggtggtggaggcacccggagaggcacccgcggaggcgcccgtggaagaggcagaggtgccggcaggaattcaaagcagcagctttcggcagaggagctggatgcccagctggacgcctataatgcgagaatggacaccagttaaacagaccagcaaatccgcgtgcggaacaggacccaggcgtctcctcttgctccctggttggggggcggtggctggggctgtgcggccaatgatggatttgtttcttttatgttttaaaataggatttaaaaactcatgtaaaggttttttttttttctttttttttttttttaattctgaaacagacctgttttgtaccgagttatttttgggataaattttactggttgctgttgtggagaaggtggcgtttccaccttttccataataaaatagaaatgtgtgtaaaaaaaaaaaaaaaaaa

SEQ ID NO: 41 - protein, 257 aa, Homo sapiens
MADKMDMSLDDIIKLNRSQRGGRGGGRGRGRAGSQGGRGGGAQAAARVNRGGGPIRNRPAIARGAAGGGGRNRPAPYSRPKQLPDKWQHDLFDSGFGGGAGVETGGKLLVSNLDFGVSDADIQELFAEFGTLKKAAVHYDRSGRSLGTADVHFERKADALKAMKQYNGVPLDGRPMNIQLVTSQIDAQRRPAQSVNRGGMTRNRGAGGFGGGGGTRRGTRGGARGRGRGAGRNSKQQLSAEELDAQLDAYNARMDTS

SEQ ID NO: 42 - DNA, 6823 bp, Homo sapiens
ggcggacaaaacgccaggcggatctcagaaggccagttcaaagacgagatcatcagatgttcattcatctggatcttcagatgcacatatggatgcatctggaccctcagatagtgatatgccaagtcggacacgacctaagagcccaagaaaacataattataggaatgaaagtgcccgtgaaagcctttgtgattctcctcatcagaatctctcaagacctcttctggaaaacaaacttaaagcattcagtattggaaaaatgagtacagctaagcgaactttaagtaaaaaggaacaggaagaattaaagaaaaaggaggatgaaaaggcagctgctgagatttatgaggagtttcttgctgcttttgaaggaagtgatggtaataaagtgaaaacatttgtgcgagggggtgttgttaatgcagctaaagaagaacatgaaacagatgaaaaaagaggtaaaatctataagccatcttcaagatttgcagatcaaaaaaatcctccaaatcagtcttccaatgaaagaccaccatctcttcttgtgatagaaaccaaaaaacctccacttaaaaaaggagagaaagaaaagaaaaaaagcaatttggaactcttcaaagaagaattaaagcaaattcaagaggaacgtgatgagagacataaaacaaaaggcagattaagtcgatttgaacctcctcagtcagattctgatggtcagcgtcgttctatggacgcgccttcaagaagaaatagatcatctggtgttcttgatgattacgcacctggctcacatgatgtaggagatccaagcactactaatttataccttggaaacattaatccacagatgaatgaagaaatgctgtgccaagaatttggaagatttggaccgttagccagtgtgaaaatcatgtggcctagaactgatgaagaaagagccagagagagaaattgcggctttgtggcctttatgaatagaagagatgctgaaagagctttaaaaaatttgaatggaaaaatgattatgtcttttgaaatgaagttaggttggggtaaagctgtacctattcctccacatccaatatacattccgccttctatgatggaacatacgcttcccccacctccatccggactgccttttaatgcgcagcctagagagcggttaaaaaaccctaatgctcctatgttaccgccacctaaaaacaaagaggattttgagaagactctgtcgcaagccatagtcaaagtggttatcccaacagaaaggaatttgctcgccctgatacatcgaatgatagagtttgttgtacgtgaagggccaatgtttgaagctatgattatgaacagagaaatcaacaatcctatgttcaggttcttatttgaaaaccagacaccagcccatgtttactataggtggaagctttattctattctgcagggagattctccaactaaatggcggacggaagattttcgtatgttcaaaaatggatctttttggaggccaccaccattaaatccgtacttgcatggaatgtcagaagagcaagaaacagaagcttttgtagaggaacctagtaaaaagggagcacttaaggaagaacagagggataaattggaagaaatcttgcggggattaactccaaggaaaaatgatattggagatgcaatggttttctgtcttaataatgctgaagctgctgaagaaatagtggattgcattactgagtcgttgtccatcttaaagacaccccttcctaaaaagattgccagattatatttggtttctgatgttttgtacaactcttcagccaaagttgctaatgcttcatattatagaaaattttttgaaacaaagttatgtcagatattttcagacctcaatgccacctatcgtacaattcaaggccatttacaatctgaaaactttaagcaacgggtaatgacttgcttcagagcatgggaagattgggcaatttatccagaaccatttttgatcaaactacaaaatattttcttaggacttgtaaatattattgaagaaaaggaaacagaggatgttccagatgaccttgatggtgcccccatcgaggaagagcttgatggtgcacctctggaagatgtagatggaattcctattgatgctactcccatcgatgatcttgatggagtccctataaaaagtcttgatgatgatcttgatggagtgcctttggatgcaactgaagactcaaaaaagaatgagcctatatttaaagttgccccatcaaaatgggaagctgtggatgaatctgaattggaagcacaggctgttacaacttctaaatgggaattatttgaccagcatgaagaatcagaagaagaagaaaatcaaaatcaagaagaagaaagtgaagatgaagaagatactcaaagttccaaatctgaagaacatcatttgtactctaatccaatcaaagaagaaatgactgagtctaagttctctaagtactctgaaatgagtgaggaaaaacgagccaaacttcgtgaaattgagctcaaagttatgaagtttcaggatgaattggaatctgggaaaagacctaaaaaaccaggccagagttttcaggagcaagtagaacactacagagataaacttcttcaacgagagaaagagaaagagttagaaagagaacgagaaagagacaagaaagataaagaaaaattggaatctcgctccaaagacaagaaggaaaaagatgagtgtactccgacaaggaaggaaaggaagaggcgacacagtacatcccccagcccatctcgcagtagcagtggtagacgagtgaaatccccatcaccaaaatcggagcgatcagagcgttcagaaagatctcataaagagagctcacggtccaggtcatctcacaaagattctcctagagatgttagcaaaaaagccaaaagatcaccatctggttcaaggacacctaaaaggtctaggcgatcacggtctagatctcctaaaaaatcaggaaagaagtccagatcccagtccagatctccacacaggtctcataaaaagtcaaagaaaaacaaacactgacgtaaatttttaagatgctgtcacttattggaaatgcgatttgttttgtgcctgaacggtctgttttttaaaaaaacaaaaaatcaaatgaaagagcattcctggggttttttgtttgtttgtgtatgcatgtgtaaactcatgagcaactgcatctgtagatctgtcattgttttatattgtgtaaattactttcattgtggctatttctcaagatgaaatttttattgttctaatggatttcatcagaaatgtgtataatggatctgctgacagtagtagtattttgttttaggatgttgtgacttagcaaaaataatacagatgtcttccccccttttgtagctttgacaatttgaattagatttcaaataaaatctgaacagaaaactataatgttgtttttttgccccaccggtgatattaagtcccttaaagtcctactgagtttcacactactgttgtgcttcttatacctgatgcactttataagccccagtgttcaagtagcttaagttttatatttactaagatgactatccaaattaagggacctgagactcctatttggtggtttgctaaccatttgcttttgataagtttctcttgggtaatactaatacccagatatcaaagactaggtagatatggcatggcgttttgttagtggaatgcctggctaaaacatttttttcacagaagcaatatgatttccatacatccaacccatgttctgagcaactacttacttttagggggaaattaaatatcttttcatttcctcttctattatgaaagaagtttatttgtaaaacaaattttctaacaaggtttggccatagaattctcttgtatgattgttgaccttttataatcttctgtaggctatctttcaaacactggcatcagaatattttttataagtttgtgtttaaacagcttagttggtccccccccccactcccaagagacttgggtttagttatagctttaagtaaaatttaaaaataaaatgtttttcaggaaacttcgtatctaatggtttgtaaattcaaggtgcaaaaagttgatttaaaccatttgcagagttgaactctattatgaaaataaatttgctacggtatgaggaagaaataaaacttgtgtaatgttggtcataatactgctataaatataataaagggttatgtagaattgaactgacactattatttgtgaatcttgatttcagttttttatgtaggcacttcatacactggtttgatgggttttttttttcctccctaaaagagaaagtagaaaactattctaacaatggattattttgatttagcttgctttttaaaaaaatcttttcaacttgttttacttaatcttgcctagtcacaaaataagatgtgcacccatggtttggagagttcctatattagctgagcagtgagatacactatttccaaacggtgcacacctacagtagctttggaaatgagccaatcactgttttacttaatggttcttatcagcatgcaaatattgcttgaaagttatttccttattcactgttttgttagtccattttgttaggaaacattaattcctaaaaatttgttcagaataattaaaagtgaacatttggtgctgatactcaaaaacctacaaatgtagccatttaaaaagtaacatgtttttctcccctgctcattgcctgggagaatggaattttatataactacctttctttgcaaaaataacggtcgtgtcgagttggtggtgattttggcattccatcttgcactggtttctagtataggcttagaaataattggtcaggtaataatctttccagtcaagttgcaagggatgcttatttctcttcaaaaaaagacatcctgcgggattgagtagaaaattttaggtcagttttgggtgcttatttgtaatatttttcctactacattggagtttagcagttctttttttctggatccagatacaagtgtcatggtttatcttacagtgggtgaaactgactttcttttggttgggtgggtgaggatttcttaggcctgatagaatatatattctgtgaagtttgttaatgtacatattagattgtattggatttttttttcttgaattgcaaatggtattattagataggttatttccagttttacttcatgacaaattacctagagtaaacctacttaatactccaatggattctatgaaagtttaatgggatcagaaattggtgacttataagggggaagatattctaccatatttttataatagcttattattcatgtttcttgtctgaaggacactcaagttacagagcaaaatttctataggttgactagaatgttcataagcatggtcttccagttgcaggaaagatcatgttctatctgtggacacttactgtcctctaccacagctacgtgccagagttgttttccacagttcttataaagggcatgacttaggctctttaccctccaacttaatgtttatacacagggattgtttactaggttaatgacatttaactcccctctcttctgtaggtgagagaaaataagtaagtcttgatctgtttcttaccaaagagagacagacctatgatggaaaatgatcacgtctctgaattttttctttaacgttatagttccttattacagatagtaagcatatgggaatttctgagctataacatgttgagaagttagaaattaaaactaacacaacaaaaggcgctgaatcaaaagatctttgcttttatttggctcagaatgtttttggcttttctgctaaagatggcagaaattactctacacagacctgatttttctttattgcagaccattcttgtgggcttaccctgagacttttatcccaattagtgaatcttggagggaatacttgcttatttatgacttaggtatttccccccaaactttaatattcttgagcacttgaaaatacttttgagaaattttaactgtgattaaatttaggtttattagaaatattctgtacacatttgcctccatggtggtgtaagttctgaaaaattatatgaccgtgacaatagtttatcatcatcattattgttattcaaaataagggtaaataaatctctgtattgccaaagtgacttaaactgttctgatgaccacacagtgtgatttctttagcagagaaagttggttttaaaaataaatagtaccacttttctaagactgtacagtttacaaataaggtttttttctttgttgttttcctcttctattaagttttagtgaaaagcctaattacagaaaattgtgcagatactagtgaagatactagtataagtttaaaggaacatgtgactgtaaaatctcacatttacaaagtgcttgatctcttcatatttcacacgcatgttttagaatagattttagggagtgtttaattcattatccttttgacttaaaatttttgttaccaacttcctaggacttagataatatataaataagtacaaatcccaggggaagtgttgtgatgctagactaaaaggtgggaatgtgctgctgttccgtgagccttgttccattgttgaaaatttgatgcctcagtgtttattcagtaccacctcatggagcttcaatgtaaatggattatatgtataattggtaatttgtatagttttgtagattgtagattaaatgcactcatcatgtc

SEQ ID NO: 43 - protein, 1028 aa, Homo sapiens
ADKTPGGSQKASSKTRSSDVHSSGSSDAHMDASGPSDSDMPSRTRPKSPRKHNYRNESARESLCDSPHQNLSRPLLENKLKAFSIGKMSTAKRTLSKKEQEELKKKEDEKAAAEIYEEFLAAFEGSDGNKVKTFVRGGVVNAAKEEHETDEKRGKIYKPSSRFADQKNPPNQSSNERPPSLLVIETKKPPLKKGEKEKKKSNLELFKEELKQIQEERDERHKTKGRLSRFEPPQSDSDGQRRSMDAPSRRNRSSGVLDDYAPGSHDVGDPSTTNLYLGNINPQMNEEMLCQEFGRFGPLASVKIMWPRTDEERARERNCGFVAFMNRRDAERALKNLNGKMIMSFEMKLGWGKAVPIPPHPIYIPPSMMEHTLPPPPSGLPFNAQPRERLKNPNAPMLPPPKNKEDFEKTLSQAIVKVVIPTERNLLALIHRMIEFVVREGPMFEAMIMNREINNPMFRFLFENQTPAHVYYRWKLYSILQGDSPTKWRTEDFRMFKNGSFWRPPPLNPYLHGMSEEQETEAFVEEPSKKGALKEEQRDKLEEILRGLTPRKNDIGDAMVFCLNNAEAAEEIVDCITESLSILKTPLPKKIARLYLVSDVLYNSSAKVANASYYRKFFETKLCQIFSDLNATYRTIQGHLQSENFKQRVMTCFRAWEDWAIYPEPFLIKLQNIFLGLVNIIEEKETEDVPDDLDGAPIEEELDGAPLEDVDGIPIDATPIDDLDGVPIKSLDDDLDGVPLDATEDSKKNEPIFKVAPSKWEAVDESELEAQAVTTSKWELFDQHEESEEEENQNQEEESEDEEDTQSSKSEEHHLYSNPIKEEMTESKFSKYSEMSEEKRAKLREIELKVMKFQDELESGKRPKKPGQSFQEQVEHYRDKLLQREKEKELERERERDKKDKEKLESRSKDKKEKDECTPTRKERKRRHSTSPSPSRSSSGRRVKSPSPKSERSERSERSHKESSRSRSSHKDSPRDVSKKAKRSPSGSRTPKRSRRSRSRSPKKSGKKSRSQSRSPHRSHKKSKKNKH

SEQ ID NO: 44 - DNA, 4105 bp, Homo sapiens
gcagacggcgctggtggtcgatcgtgtggcgccggaggacgttccccgcggccggagccatggagatgccgctgccccccgatgaccaggagcttcgaaatgtcatcgacaagctcgcccagttcgtggctcgcaatgggcccgagtttgagaagatgactatggagaagcagaaggacaaccccaaattctcgtttcttttcggaggcgaattctacagttactacaagtgcaagctggcgctggagcagcagcagctcatctgcaagcagcagaccccggagctggagccagccgccaccatgccacccctgccacagcccccgctggcccccgccgcgcccatcccgccggcccagggcgcgccatccatggacgagctcatccagcagagccagtggaacctccagcagcaggagcagcacttgctggcgctcagacaggagcaagtgacagcggccgtggcccacgcggtggagcagcagatgcagaagcttctggaggagacccagctagacatgaacgagtttgacaacctcctgcagcccatcatcgacacgtgcaccaaggacgccatctcggccgggaagaactggatgttcagcaatgccaagtccccgccgcactgtgagctgatggccggccacctccggaaccgcatcacggctgatggggcacacttcgagctgcggctgcacctcatctacctgatcaatgacgtgctgcaccactgccagcgcaagcaggcccgggagctgctggccgccctgcagaaggtcgtggtgcccatctactgcaccagcttcttggccgtggaggaagacaagcagcagaagatcgcccggctcctgcagctctgggagaaaaacggctacttcgatgactccatcattcagcagctacagagcccagccctggggcttggtcagtaccaggccaccctcatcaacgagtactcctcagtggtccagccggtgcagctggccttccagcagcagatccagaccctcaagacgcagcacgaggagtttgtcaccagcctggcccagcagcagcagcagcagcaacagcagcagcagcagctccagatgccgcagatggaggctgaagtcaaggccacgcctccaccgcctgctccacccccggccccagcacctgcccctgccatcccgcccaccacccagcctgatgacagcaagcctcccatccagatgcctggctcttcagagtacgaagctccaggaggggtccaggatcctgcagctgccggcccccggggccccgggccacacgaccagatcccaccaaacaagcccccttggtttgaccagcctcaccccgtggctccttggggccagcagcagccgccagagcagccaccctacccgcaccaccagggcggcccaccccactgccccccctggaacaacagccatgagggcatgtggggcgagcagcgcggtgaccccggctggaacggccagcgcgacgcgccctggaacaaccagcccgacgccgcctggaacagccagttcgagggcccctggaacagccagcacgagcagccgccctggggcgggggccagcgcgagccacccttccgcatgcagcggcccccacacttccgggggcccttcccgccccaccagcagcacccgcagttcaaccagcctccgcacccccacaacttcaaccgcttcccgccccgcttcatgcaggacgacttcccgccacggcaccccttcgagcggccgccctatccccaccgcttcgactacccccagggggacttccctgccgaaatggggccccctcaccaccaccctggccaccgcatgcctcatcctggcatcaacgagcacccgccttgggctggaccccagcaccctgacttcggccctcccccccatggcttcaacgggcagcccccacacatgcggcgacagggcccgccccacatcaaccacgatgaccccagcctggtccccaatgtgccctacttcgatctccctgctgggctgatggcccccctcgtgaagctggaagatcacgagtacaagcctttggaccctaaagacatccgcctcccaccccccatgccgcccagcgagaggctgctggctgcagtggaggccttctacagccccccgtcccacgacaggcccaggaacagtgaaggctgggagcagaacggcctctatgagttcttccgagcaaaaatgcgggcccggcggaggaaaggccaggagaagaggaacagcggaccctcgaggtctcggagcagatccaagagtcgagggcgttcttcctcccgctccaactcaagatcctccaagtcttcaggctcgtactcaaggtcaaggtcgcgctcctgctcccgttcctactcccgctccagatctagaagtcggagcaggtcgcgctcctccagaagccgctcccggtcccagtcgcggtcccggtccaagtcgtactccccaggaagaagacgccggtcacggtccaggagccccaccccgccttcctctgctggtctgggttctaattcggcgcctcccattcctgactcaaggctcggagaagagaacaaaggccatcagatgctggtgaagatgggctggagcggctcaggcggcctcggtgcgaaggagcaagggatccaggaccccatcaagggcggggacgtccgggataagtgggaccagtataaaggcgtgggcgtggctctggatgacccctatgagaactaccgcaggaacaagagctactccttcatcgcccgcatgaaggccagggacgagtgtaagtaggcgcccatgccgggagccgcgccggtggccagcggtgccggctgtgggaccttcctggctgactggcagaggaagattgcagtgacagctcagttcttacaccgtctccacttgtggagagccacaggaagagaaggaagaccagcgcatgcccagtgggaacgccgtgctccatggcgtggagggcacgggtgccacccacaaaccacaccaggaggcgctgcagccaccagggccccaggctcgttttcctttatagcgaagcaaaaaacacaagaccctccgcccgagtaaattcttcagccacgaagggatggatgtgcatctgccctaagtcagattgaagcctcctcctaggctccaggaggagcatgtgcaggaagggttggcctgagccagagccggcaccccagctccttcctccagctccccgcacccaccggccctgacctggcttcccctcggctgtgtagggacaaagccgaagcccagtgccatgactgcccgcggattgaaaaacagaaacacaaaactttgacttgacttgcgaagtgaagcagggttttcatttttactctccttggtttaggtctagaaagaagaatactgacctgagaggaggcccagtgagattctgaaacctaattcttcgaaggcgtactggcccttagttcagttattttagttgtataagttgatattttttttctggaatgtagccatttgctgttatctgggaaacaagattctaacaggaaaccagcctaagacacttcaggttgagcgctgcctcggagtctgtgcccgtcgcgtcccctgcttgagttttgcacttggaagaaccctgcaccggctggcgtgtgcgacggcccagtcccatccagagcatggagcccgaccccagccagcgccttccactccatcatttcatttcacacccccgaagggaggggaggccaggaggggagctgctcctgccagaagctggtgggtgaccgttgggaatcggccacacctggtgtccatgggcagcctggtgcaattccattcatttgtacagaaacatttttgaaaaattcttttcaataagatgcaaaatcttccaacttttcaacccaacgtgatgaatatttgattttgttctagatttcctgtagctgtgaattgttaaaatgtatgattcaggataaaacgtaaacacgtgctgttagtaatttcttgtggatttcattgttttgccttcaaataaagcctttttttaaagaaaaaaaaaaaaaaaaaaaaaaaaaaaaaaa

SEQ ID NO: 45 - protein, 884 aa, Homo sapiens
MTMEKQKDNPKFSFLFGGEFYSYYKCKLALEQQQLICKQQTPELEPAATMPPLPQPPLAPAAPIPPAQGAPSMDELIQQSQWNLQQQEQHLLALRQEQVTAAVAHAVEQQMQKLLEETQLDMNEFDNLLQPIIDTCTKDAISAGKNWMFSNAKSPPHCELMAGHLRNRITADGAHFELRLHLIYLINDVLHHCQRKQARELLAALQKVVVPIYCTSFLAVEEDKQQKIARLLQLWEKNGYFDDSIIQQLQSPALGLGQYQATLINEYSSVVQPVQLAFQQQIQTLKTQHEEFVTSLAQQQQQQQQQQQQLQMPQMEAEVKATPPPPAPPPAPAPAPAIPPTTQPDDSKPPIQMPGSSEYEAPGGVQDPAAAGPRGPGPHDQIPPNKPPWFDQPHPVAPWGQQQPPEQPPYPHHQGGPPHCPPWNNSHEGMWGEQRGDPGWNGQRDAPWNNQPDAAWNSQFEGPWNSQHEQPPWGGGQREPPFRMQRPPHFRGPFPPHQQHPQFNQPPHPHNFNRFPPRFMQDDFPPRHPFERPPYPHRFDYPQGDFPAEMGPPHHHPGHRMPHPGINEHPPWAGPQHPDFGPPPHGFNGQPPHMRRQGPPHINHDDPSLVPNVPYFDLPAGLMAPLVKLEDHEYKPLDPKDIRLPPPMPPSERLLAAVEAFYSPPSHDRPRNSEGWEQNGLYEFFRAKMRARRRKGQEKRNSGPSRSRSRSKSRGRSSSRSNSRSSKSSGSYSRSRSRSCSRSYSRSRSRSRSRSRSSRSRSRSQSRSRSKSYSPGRRRRSRSRSPTPPSSAGLGSNSAPPIPDSRLGEENKGHQMLVKMGWSGSGGLGAKEQGIQDPIKGGDVRDKWDQYKGVGVALDDPYENYRRNKSYSFIARMKARDECK

SEQ ID NO: 46 - DNA, 2441 bp, Homo sapiens
atgtccaagcggcaccggttggacctaggggaggattacccctctggcaagaagcgtgcggggaccgatgggaaggatcgagatcgagaccgggatcgtgaagatcggtctaaagatcgagaccgagaacgtgatagaggagatagagagcgagagagggagaaagaaaaggagaaggagttgcgagcttcaacaaatgctatgcttatcagtgctggattaccacctttgaaagcttcccattcagctcactcaacccactcagcacattcaacgcattctacacattctgctcattcaacgcatgccggacatgcaggtcacacgtcacttccacagtgcattaatccgttcaccaacttaccccatactcctcgatactatgatattctaaagaaacgtcttcagctccctgtttgggaatacaaggataggtttacagatattctggttagacatcagtcctttgtactggttggtgagactgggtctggtaaaacaacacagattccacagtggtgtgtggagtacatgcgatcattaccaggacccaagagaggagttgcctgtacccaacccaggagagtggctgcaatgagtgtggctcagagagttgctgatgagatggatgtgatgttgggccaggaggttggttactccattcgatttgaagactgcagtagtgcaaaaaccattcttaagtatatgactgatgggatgttacttcgtgaagctatgaatgatcccctcctggagcgttatggtgtaataattcttgatgaggctcatgagaggacactggctacagatattctaatgggtgttctgaaggaagttgtaagacagagatcagatttaaaggttatagttatgagcgctactctagatgcaggaaaattccagatttactttgataactgtcctctcctaactattcctgggcgtacacatcctgttgagatcttctatactccagaaccagagagagattatcttgaagcagcaattcgaacagttatccagattcatatgtgtgaagaggaagagggagatcttcttcttttcttaactggtcaagaggaaattgatgaagcctgtaagagaataaagcgtgaagttgatgatttgggccctgaagttggtgacattaaaatcattccattgtattctacacttccacctcagcagcagcaacgcatttttgagcctccacctcccaaaaaacagaatggagcaattggaagaaaggtagttgtgtcaactaacatagcagagacgtctttgacaatagatggtgtggtgtttgtgattgatcctggatttgcgaaacagaaggtctacaatcctcgaatcagagttgagtcccttttggtgacagctattagtaaagcttcagctcagcaaagggctggtcgagctggacgtaccagacctggaaaatgcttcagactttacacagagaaagcttataaaacagaaatgcaggataacacctatcctgagattttgcgttctaatttaggatcagttgtgttacaattgaagaaacttggtattgatgacttggtacattttgattttatggatccaccagctcctgaaactctgatgagagccctggaacttttgaattacctggctgctttaaatgatgatggagatctgactgaattgggatccatgatggcagagtttcctctagatccacagctcgcaaaaatggttattgcaagttgtgactacaactgttctaatgaggtcctatctattactgctatgttgtcagtcccacagtgttttgttcgccccacggaggccaagaaagccgcagatgaggccaagatgagatttgcccacatagatggagatcatctgacactgctgaacgtctaccatgcttttaaacaaaatcatgaatcggttcagtggtgttatgacaacttcattaactacaggtccctgatgtccgcagacaatgtacgccagcagctatctcgaattatggacagatttaatttgcctcgtcgaagtactgactttacaagcagggactattatattaatataagaaaagctttggttactgggtattttatgcaggtggcacatttagaacgaacagggcattacttaactgtgaaagataaccaggtggttcagttgcatccctctactgttcttgaccacaaacctgaatgggtgctttataatgagtttgttctaacaacaaagaattacatccggacatgtacagatatcaagccagaatggttggtgaaaattgcccctcaatattatgacatgagcaatttcccacagtgtgaagcaaagagacagttggaccgcatcattgccaaacttcaatccaaggaatattcacagtactgaattcagtgcttagaactgaagttattgagaggacagctttaaaagatgaatga

SEQ ID NO: 47 - protein, 795 aa, Homo sapiens
MSKRHRLDLGEDYPSGKKRAGTDGKDRDRDRDREDRSKDRDRERDRGDREREREKEKEKELRASTNAMLISAGLPPLKASHSAHSTHSAHSTHSTHSAHSTHAGHAGHTSLPQCINPFTNLPHTPRYYDILKKRLQLPVWEYKDRFTDILVRHQSFVLVGETGSGKTTQIPQWCVEYMRSLPGPKRGVACTQPRRVAAMSVAQRVADEMDVMLGQEVGYSIRFEDCSSAKTILKYMTDGMLLREAMNDPLLERYGVIILDEAHERTLATDILMGVLKEVVRQRSDLKVIVMSATLDAGKFQIYFDNCPLLTIPGRTHPVEIFYTPEPERDYLEAAIRTVIQIHMCEEEEGDLLLFLTGQEEIDEACKRIKREVDDLGPEVGDIKIIPLYSTLPPQQQQRIFEPPPPKKQNGAIGRKVVVSTNIAETSLTIDGVVFVIDPGFAKQKVYNPRIRVESLLVTAISKASAQQRAGRAGRTRPGKCFRLYTEKAYKTEMQDNTYPEILRSNLGSVVLQLKKLGIDDLVHFDFMDPPAPETLMRALELLNYLAALNDDGDLTELGSMMAEFPLDPQLAKMVIASCDYNCSNEVLSITAMLSVPQCFVRPTEAKKAADEAKMRFAHIDGDHLTLLNVYHAFKQNHESVQWCYDNFINYRSLMSADNVRQQLSRIMDRFNLPRRSTDFTSRDYYINIRKALVTGYFMQVAHLERTGHYLTVKDNQVVQLHPSTVLDHKPEWVLYNEFVLTTKNYIRTCTDIKPEWLVKIAPQYYDMSNFPQCEAKRQLDRIIAKLQSKEYSQY

SEQ ID NO: 48 - DNA, 2225 bp, Homo sapiens
cgacatggcgcgcgagctgcgggcgctgctgctgtggggccgccgcctgcggcctttgctgcgggcgccggcgctggcggccgtgccgggaggaaaaccaattctgtgtcctcggaggaccacagcccagttgggccccaggcgaaacccagcctggagcttgcaggcaggacgactgttcagcacgcagaccgccgaggacaaggaggaacccctgcactcgattatcagcagcacagagagcgtgcagggttccacttccaaacatgagttccaggccgagacaaagaagcttttggacattgttgcccggtccctgtactcagaaaaagaggtgtttatacgggagctgatctccaatgccagcgatgccttggaaaaactgcgtcacaaactggtgtctgacggccaagcactgccagaaatggagattcacttgcagaccaatgccgagaaaggcaccatcaccatccaggatactggtatcgggatgacacaggaagagctggtgtccaacctggggacgattgccagatcggggtcaaaggccttcctggatgctctgcagaaccaggctgaggccagcagcaagatcatcggccagtttggagtgggtttctactcagctttcatggtggctgacagagtggaggtctattcccgctcggcagccccggggagcctgggttaccagtggctttcagatggttctggagtgtttgaaatcgccgaagcttcgggagttagaaccgggacaaaaatcatcatccacctgaaatccgactgcaaggagttttccagcgaggcccgggtgcgagatgtggtaacgaagtacagcaacttcgtcagcttccccttgtacttgaatggaaggcggatgaacaccttgcaggccatctggatgatggaccccaaggatgtcggtgagtggcaacatgaggagttctaccgctacgtcgcgcaggctcacgacaagccccgctacaccctgcactataagacggacgcaccgctcaacatccgcagcatcttctacgtgcccgacatgaaaccgtccatgtttgatgtgagccgggagctgggctccagcgttgcactgtacagccgcaaagtcctcatccagaccaaggccacggacatcctgcccaagtggctgcgcttcatccgaggtgtggtggacagtgaggacattcccctgaacctcagccgggagctgctgcaggagagcgcactcatcaggaaactccgggacgttttacagcagaggctgatcaaattcttcattgaccagagtaaaaaagatgctgagaagtatgcaaagttttttgaagattacggcctgttcatgcgggagggcattgtgaccgccaccgagcaggaggtcaaggaggacatagcaaagctgctgcgctacgagtcctcggcgctgccctccgggcagctaaccagcctctcagaatacgccagccgcatgcgggccggcacccgcaacatctactacctgtgcgcccccaaccgtcacctggcagagcactcaccctactatgaggccatgaagaagaaagacacagaggttctcttctgctttgagcagtttgatgagctcaccctgctgcaccttcgtgagtttgacaagaagaagctgatctctgtggagacggacatagtcgtggatcactacaaggaggagaagtttgaggacaggtccccagccgccgagtgcctatcagagaaggagacggaggagctcatggcctggatgagaaatgtgctggggtcgcgtgtcaccaacgtgaaggtgaccctccgactggacacccaccctgccatggtcaccgtgctggagatgggggctgcccgccacttcctgcgcatgcagcagctggccaagacccaggaggagcgcgcacagctcctgcagcccacgctggagatcaaccccaggcacgcgctcatcaagaagctgaatcagctgcgcgcaagcgagcctggcctggctcagctgctggtggatcagatatacgagaacgccatgattgctgctggacttgttgacgaccctagggccatggtgggccgcttgaatgagctgcttgtcaaggccctggagcgacactgacagccagggggccagaaggactgacaccacagatgacagccccacctccttgagctttatttacctaaatttaaaggtatttcttaacccgaaaaaaaaaaaaaaa

SEQ ID NO: 49 - protein, 704 aa, Homo sapiens
MARELRALLLWGRRLRPLLRAPALAAVPGGKPILCPRRTTAQLGPRRNPAWSLQAGRLFSTQTAEDKEEPLHSIISSTESVQGSTSKHEFQAETKKLLDIVARSLYSEKEVFIRELISNASDALEKLRHKLVSDGQALPEMEIHLQTNAEKGTITIQDTGIGMTQEELVSNLGTIARSGSKAFLDALQNQAEASSKIIGQFGVGFYSAFMVADRVEVYSRSAAPGSLGYQWLSDGSGVFEIAEASGVRTGTKIIIHLKSDCKEFSSEARVRDVVTKYSNFVSFPLYLNGRRMNTLQAIWMMDPKDVGEWQHEEFYRYVAQAHDKPRYTLHYKTDAPLNIRSIFYVPDMKPSMFDVSRELGSSVALYSRKVLIQTKATDILPKWLRFIRGVVDSEDIPLNLSRELLQESALIRKLRDVLQQRLIKFFIDQSKKDAEKYAKFFEDYGLFMREGIVTATEQEVKEDIAKLLRYESSALPSGQLTSLSEYASRMRAGTRNIYYLCAPNRHLAEHSPYYEAMKKKDTEVLFCFEQFDELTLLHLREFDKKKLISVETDIVVDHYKEEKFEDRSPAAECLSEKETEELMAWMRNVLGSRVTNVKVTLRLDTHPAMVTVLEMGAARHFLRMQQLAKTQEERAQLLQPTLEINPRHALIKKLNQLRASEPGLAQLLVDQIYENAMIAAGLVDDPRAMVGRLNELLVKALERH

SEQ ID NO: 50 - DNA, 1853 bp, Homo sapiens
atcgcgcgagacagcggaaggagcaagagtgggaggcgcgcgcggaggccgcgacggacgcaagatggcgacggcgaccatagctctccaggtcaatggccagcaaggaggggggtccgagccggcggcggcggcggcagtggtggcagcgggagacaaatggaaacctccacagggcacagactccatcaagatggagaacgggcagagcacagccgccaagctggggctgcctcccctgacgcccgagcagcaggaggcccttcagaaggccaagaagtacgccatggagcagagcatcaagagtgtgctggtgaagcagaccatcgcgcaccagcagcagcagctcaccaacctgcagatggcggctcagcggcagcgggcgctggccatcatgtgccgcgtctacgtgggctctatctactatgagctgggggaggacaccatccgccaggcctttgccccctttggccccatcaagagcatcgacatgtcctgggactccgtcaccatgaagcacaagggctttgccttcgtggagtatgaggtccccgaagctgcacagctggccttggagcagatgaactcggtgatgctggggggcaggaacatcaaggtgggcagacccagcaacatagggcaggcccagcccatcatagaccagttggctgaggaggcacgggccttcaaccgcatctacgtggcctctgtgcaccaggacctctcagacgatgacatcaagagcgtgtttgaggcctttggcaagatcaagtcctgcacactggcccgggaccccacaactggcaagcacaagggctacggcttcattgagtacgagaaggcccagtcgtcccaagatgctgtgtcttccatgaacctctttgacctgggtggccagtacttgcgggtgggcaaggctgtcacaccgcccatgcccctactcacaccagccacgcctggaggcctcccacctgccgctgctgtggcagctgctgcagccactgccaagatcacagctcaggaagcagtggccggagcagcggtgctgggtaccctgggcacacctggactggtgtccccagcactgaccctggcccagcccctgggcactttgccccaggctgtcatggctgcccaggcacctggagtcatcacaggtgtgaccccagcccgtcctcctatcccggtcaccatcccctcggtgggagtggtgaaccccatcctggccagccctccaacgctgggtctcctggagcccaagaaggagaaggaagaagaggagctgtttcccgagtcagagcggccagagatgctgagcgagcaggagcacatgagcatctcgggcagtagcgcccgacacatggtgatgcagaagctgctccgcaagcaggagtctacagtgatggttctgcgcaacatggtggaccccaaggacatcgatgatgacctggaaggggaggtgacagaggagtgtggcaagttcggggccgtgaaccgcgtcatcatctaccaagagaaacaaggcgaggaggaggatgcagaaatcattgtcaagatctttgtggagttttccatagcctctgagactcataaggccatccaggccctcaatggccgctggtttgctggccgcaaggtggtggctgaagtgtacgaccaggagcgttttgataacagtgacctctctgcgtgacagtggtccctctccccggacttgcacttgttccttgtttcctctgggttttatagtgatacagtggtgtccccggggccaggcgcgctctgcccagcccagcctacagtgcggataaaggtgcggatgctgctggccctgaaaaaaaaaaaaaaaaaaa

SEQ ID NO: 51 - protein, 542 aa, Homo sapiens
MATATIALQVNGQQGGGSEPAAAAAVVAAGDKWKPPQGTDSIKMENGQSTAAKLGLPPLTPEQQEALQKAKKYAMEQSIKSVLVKQTIAHQQQQLTNLQMAAQRQRALAIMCRVYVGSIYYELGEDTIRQAFAPFGPIKSIDMSWDSVTMKHKGFAFVEYEVPEAAQLALEQMNSVMLGGRNIKVGRPSNIGQAQPIIDQLAEEARAFNRIYVASVHQDLSDDDIKSVFEAFGKIKSCTLARDPTTGKHKGYGFIEYEKAQSSQDAVSSMNLFDLGGQYLRVGKAVTPPMPLLTPATPGGLPPAAAVAAAAATAKITAQEAVAGAAVLGTLGTPGLVSPALTLAQPLGTLPQAVMAAQAPGVITGVTPARPPIPVTIPSVGVVNPILASPPTLGLLEPKKEKEEEELFPESERPEMLSEQEHMSISGSSARHMVMQKLLRKQESTVMVLRNMVDPKDIDDDLEGEVTEECGKFGAVNRVIIYQEKQGEEEDAEIIVKIFVEFSIASETHKAIQALNGRWFAGRKVVAEVYDQERFDNSDLSA

SEQ ID NO: 52 - DNA, 2227 bp, Homo sapiens
gacgacctgtctcgccgagcgcacgcttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaagaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagtt

SEQ ID NO: 53 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 54 - DNA, 2709 bp, Homo sapiens
ccctgaccccgcgccgccgagcgccctgaggactcagcgaagggtgggcgccgccgaggcctcctgccgctggcgggtttccgcggagtgccgcccggctccgctctgccgccggcgcggctcatgggcagagtcggccgggcgggccggcattaaactgaagaaaagatgtccctgtacgatgacctaggagtggagaccagtgactcaaaaacagaaggctggtccaaaaacttcaaacttctgcagtctcagcttcaggtgaagaaggcagctctcactcaggcaaagagccaaaggacgaaacaaagtacagtcctcgccccagtcattgacctgaagcgaggtggctcctcagatgaccggcaaattgtggacactccaccgcatgtagcagctgggctgaaggatcctgttcccagtgggttttctgcaggggaagttctgattcccttagctgacgaatatgaccctatgtttcctaatgattatgagaaagtagtgaagcgccaaagagaggaacgacagagacagcgggagctggaaagacaaaaggaaatagaagaaagggaaaaaaggcgtaaagacagacatgaagcaagtgggtttgcaaggagaccagatccagattctgatgaagatgaagattatgagcgagagaggaggaaaagaagtatgggcggagctgccattgccccacccacttctctggtagagaaagacaaagagttaccccgagattttccttatgaagaggactcaagacctcgatcacagtcttccaaagcagccattcctcccccagtgtacgaggaacaagacagaccgagatctccaaccggacctagcaactccttcctcgctaacatggggggcacggtggcgcacaagatcatgcagaagtacggcttccgggagggccagggtctggggaagcatgagcagggcctgagcactgccttgtcagtggagaagaccagcaagcgtggcggcaagatcatcgtgggcgacgccacagagaaagatgcatccaagaagtcagattcaaatccgctgactgaaatacttaagtgtcctactaaagtggtcttactaaggaacatggttggtgcgggagaggtggatgaagacttggaagttgaaaccaaggaagaatgtgaaaaatatggcaaagttggaaaatgtgtgatatttgaaattcctggtgcccctgatgatgaagcagtacggatatttttagaatttgagagagttgaatcagcaattaaagcggttgttgacttgaatgggaggtattttggtggacgggtggtaaaagcatgtttctacaatttggacaaattcagggtcttggatttggcagaacaagtttgattttaagaactagagcacgagtcatctccggtgatccttaaatgaactgcaggctgagaaaagaaggaaaaaggtcacagcctccatggctgttgcataccaagactcttggaaggacttctaagatatatgttgattgatcccttttttattttgtggttttttaatatagtataaaaatccttttaaaaaaacaacaatctgtgtgcctctctggttgtttctcttttttattattactcctgagttgatgacattttttgttagatttcatggtaattctcaagtgcttcaatgatgcagcatttcttgcactaaaaaaaaaaaaaaaaaaaaaactagaaagttttgggacatggggttatattaaattattctttgtttttctttttcttttaataaagcctgcaagttactaaattgtagtttcataaattctgtagtaaagtatcatcttggcagtgtgccaaaggtgaaaatgatgctttctctaacagagaaattcttagtgactccagtcgtagaaaaacgtctttacaacctgaataagattgaagaattgtgaacataccatggcctattggatgaatcatttgccgtaggctaaatcagactgtagggtttgtgatggatttatggagtatgtgggtatagaaatcatgaatctagcatttgttttcagagattcaagcatagtcttaagggtagatcagaaatgacaaatgaattcaaaacctagcaggtgcattgtaaatgtgtgcccagttatgttttggaaatggcagttccttggggtcatgtttctactggcaaaatttgcaatagtgttctattgtatgtaattttaaaatttataagattatccacgttggccaagtaaactgtactgccaatagaattctggaattgtgagaaattgtatcattgaagttcagtaggatgtgtggcttaaaaatttatcaggaccacaaaaaagaaaacaaaaatatttggtactgaggttcattgccagggcaggaggtatttccagaaaatactcatgcctgtgttctgttccttgctttcccaaatactgcatgtgactttcctaagcggcagctgaaagactcgagcccgtgctgtctcctttggttattatgacatgaaagtgtatcaagaactcagcatttctttgcatccatggacttggtttggagacataaggaatattctgaccctttttaaaaaaggattttctcatgtttttatttaacataaataaaagaataacattttatcttttgtggtattattttattgaataaaattgagttttatgataaaaaaaaaaaaaaa

SEQ ID NO: 55 - protein, 401 aa, Homo sapiens
MSLYDDLGVETSDSKTEGWSKNFKLLQSQLQVKKAALTQAKSQRTKQSTVLAPVIDLKRGGSSDDRQIVDTPPHVAAGLKDPVPSGFSAGEVLIPLADEYDPMFPNDYEKVVKRQREERQRQRELERQKEIEEREKRRKDRHEASGFARRPDPDSDEDEDYERERRKRSMGGAAIAPPTSLVEKDKELPRDFPYEEDSRPRSQSSKAAIPPPVYEEQDRPRSPTGPSNSFLANMGGTVAHKIMQKYGFREGQGLGKHEQGLSTALSVEKTSKRGGKIIVGDATEKDASKKSDSNPLTEILKCPTKVVLLRNMVGAGEVDEDLEVETKEECEKYGKVGKCVIFEIPGAPDDEAVRIFLEFERVESAIKAVVDLNGRYFGGRVVKACFYNLDKFRVLDLAEQV

SEQ ID NO: 56 - DNA, 1108 bp, Homo sapiens
atgtcccacggccccaagcagcccggcgcgtcctccgcgccggcgggcggcaaggctccgggccagcatgggggcttcgtggtgactgtcaagcaagagcgcggcgagggtccacgcgcgggcgagaaggggtcccacgaggaggagccggtgaagaaacgcggctggcccaagggcaagaagcggaagaagattctgccgaatgggcccaaggcaccggtcacgggctacgtgcgcttcctgaacgagcggcgcgagcagatccgcacgcgccacccggatctgccctttcccgagatcaccaagatgctgggcgccgagtggagcaagctgcagccaacggaaaagcagcggtacctggatgaggccgagagagagaagcagcagtacatgaaggagctgcgggcgtaccagcagtctgaagcctataagatgtgcacggagaagatccaggagaagaagatcaagaaagaagactcgagctctgggctcatgaacactctcctgaatggacacaagggtggggactgcgatggcttctccaccttcgatgttcccatcttcactgaagagttcttggaccaaaacaaagcgcgtgaggcggagcttcggcgcttgcggaagatgaatgtggccttcgaggagcagaacgcggtactgcagaggcacacgcagagcatgagcagcgcgcgcgagcgtctggagcaggagctggcgctggaggagcggaggacgctggcgctgcagcagcagctccaggccgtgcgccaggcgctcaccgccagcttcgcctcactgccggtgccgggcacgggcgaaacgcccacgctgggcactctggacttctacatggcccggcttcacggagccatcgagcgcgaccccgcccagcacgagaagctcatcgtccgcatcaaggaaatcctggcccaggtcgccagcgagcacctgtgaggagtgggcgggcccacgatgcagaggagaagctgtgggcgcggccctgccacaccccaccccgtggacgagaggctgggggtccaccctttggggcctggtcccatcctgcacctttgggggctccagcccccctaaaattaaatttctgcag

SEQ ID NO: 57 - protein, 317 aa, Homo sapiens [sequence omitted]

SEQ ID NO: 58 - DNA, 1428 bp, Homo sapiens
cgagctgataaaggcgccattttggaggggccgcgggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgataaattgtattttaagtgatgtcatagacaggattgtttaaatttagttaactccatactcttcagactgtgatattgtgtaaatgtctatctgccctggtttgtgtgaactgggatgttgggggtgtttgtggttatcttacctggggaagttcttatgtttatcttgcttttcatgtgtctttctgtagacatatctgaagagatggattaagaatgctttggattaaggattgtggagcacatttcaatcattttaggattgtcaaaaggaggattgaggaggatcagatcaataatggaggcaatggtatgactccaagtgctattgtcacagatgaaattggcagtattgaccttatactaaaaggcaggggctaaaaa

SEQ ID NO: 59 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 60 - DNA, 1088 bp, Homo sapiens
ccgaagcttccggtggccggcttagttaggagctatggctaaacatcatcctgatttgatcttttgccgcaagcaggctggtgttgccatcggaagactgtgtgaaaaatgtgatggcaagtgtgtgatttgtgactcctatgtgcgtccctgcactctggtgcgcatatgtgatgagtgtaactatggatcttaccaggggcgctgtgtgatctgtggaggacctggggtctctgatgcctattattgtaaggagtgcaccatccaggagaaggacagagatggctgcccaaagattgtcaatctggggagctctaagacagacctcttctatgaacgcaaaaaatacggcttcaagaagaggtgattggtgggtggccccttcctccccccaacatcagtctgctgcagctgccagaaaacatgcctactactaccagcagaaagggagcagagcccagagcatcaccaggagtgcctgctagtgtactggcagcttgccaccccctcctctcccttcacccagacacgtggtagggatggaaaaggattcttcacagagcactctggcacaccatatcggagaaaacttgatagattagttaatggtttttcttgaattcgagaagcaaagatctgttctccatattggtatgttctccctcaaccaagatcttctaaaaagaaataatattttagtcttctgcttgaggaactgactgtgaagcgacgcccagtgaaaaacatgttcttgcagcagctctggtggcagctgtccttgaggaacctttggtgtgtggtgggaagctatcagaacaagaaatgtaggcatttcccgttttttttggggggggggtgggggggcagggctctgccctcttgaaaggcatttacttgtttaacacttgtccagctacagtggggtacagtagctggctattcacaggcatcatcatagcccactagtctcatattattttccttttgagaaattggaaactctttctgttgctattatattaataaagttggtgtttattttctggtaaaaaaaaaaaaaaaaaaaaaaaaaaaaaaaaaaaaaaaaa

SEQ ID NO: 61 - protein, 110 aa, Homo sapiens
MAKHHPDLIFCRKQAGVAIGRLCEKCDGKCVICDSYVRPCTLVRICDECNYGSYQGRCVICGGPGVSDAYYCKECTIQEKDRDGCPKIVNLGSSKTDLFYERKKYGFKKR

SEQ ID NO: 62 - DNA, 786 bp, Homo sapiens
tcttctgcgacggcgcggacctggagcttccgcgcggtggcttcactctcctgtaaaacgctagagcggcgagttgttacctgcgtcctctgacctgagagcgaaggggaaagcggcgagatgactgaccgctacaccatccatagccagctggagcacctgcagtccaagtacatcggcacgggccacgccgacaccaccaagtgggagtggctggtgaaccaacaccgcgactcgtactgctcctacatgggccacttcgaccttctcaactacttcgccattgcggagaatgagagcaaagcgcgagtccgcttcaacttgatggaaaagatgcttcagccttgtggaccgccagccgacaagcccgaggagaactgagactctgccttaccaccgcagtgcggggcacctctcccagcgtttctccggtttgccaatcctcttaagtattcctgtctccaaaggaccggctctccatggctcctgcgcctcgtgctttccgcgtacagaagtgcttgcccggggagtcccgcctgacctgccttcatgtggacccttagaacagcactgggagaccagcaggactcctgagaactgtgctggtggagaggtcctagagccggcgagcgtttgagaagagggcatggcgctggagtgagatgggatttggcgtctcgtttttggctaattgattgtcattggctttttccataaagtttagaaatcgttcagtcaaaaaaaaaaaaaaaaaaaaaaaaaaaaaaaaaaaaaaaaaaaaaaaaa

SEQ ID NO: 63 - protein, 86 aa, Homo sapiens
MTDRYTIHSQLEHLQSKYIGTGHADTTKWEWLVNQHRDSYCSYMGHFDLLNYFAIAENESKARVRFNLMEKMLQPCGPPADKPEEN

SEQ ID NO: 64 - DNA, 5136 bp, Homo sapiens
accttcgcggccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaatcagttaaataagacttgtctcggtcacctgtgccctgtccagactcgaggcagtggtaacactgcacagtgctatgtggcttctctttgaggatttttgggttttgtaactaaattcttgctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatccattttatccattttaactcatgtaatcctcatgtgagtattcctgtttaacacttgagtaaactgaggcacagagaacataagttgcatgccatagtcacacactgtgaaagtgaaaagagaatgtgtgcaaaacacgtcacagtcctggtttctgagtaaaggcaggctgttatctttagaatcaagctatcacagggagataggcaatgctgtgggtgttggaggaaggtgagagcctgttgctaacaatttcctggttttaaagctaaggctgattttattgggaagatctcacatgtgtgtggcccctgagagttcccagtgccttttatttgcagtccttccatttggacctcctagctgccccatcaggtcatctccagggctcagaggggtgagaccatttcccaaggtcacagaaccagctctctagtcaccaccctgcctctccctctcacccagagtcagtaccagttttatggctttattacaaactgctgggtccctcccattttcaacttgattgatgggatgtcatcccttatcctgtctgacatttgcctctggcctggttgctagaagtttgccccaggggcaagagttgaaatttggcttcctgaggtgggctttgtggtttgcgtccctaaagtgagcccactactggttgcttgtccatggccaacaccagaaatcccctgagcactacctgggtctcattccaagaaggaagagggtcaggagacctggggagtctcatattccaagttcttctttctttctgggagcagtgggcagttcatggtgttagggcactcacccccacagactggcaaaccctgcaggacttccgtggctgaggctgtgaccggaggccaggaatgccgttgggtggattgtgagtgaatgggccctttgagctgccctctagagagcaaatccagtttcctggagctcctgaatgaatatctgtactggctcgctcagatgcagaagctccattgaccatgaggccttgtgaacatcagtggccacaggcccagtgtgctgcttggcactgcactagtttaggacctgcagcatgtaggtagcgtcctagtgtttataatacaaagctgctctgcacagcttttctgattcttcttgcaatctcctgaggattatctgccccatttttaaaacgaggtggaatacccaaggtcatgtagccagtgagtgctctggaaagccaaagcagctcatcccttcctggggaccacactgctctgctccaccagaccacactatgaaataggaataagtgctcctgttgcaggactgctgggaaaacaggtggtgtgggacttaagtcaccataattttgaagacttgcatgcagagggctccaggaattgtagacattaaggaatttcactttcagttctacccactacttaagtacttgtcatgtactcttagaggaggccagtaatgatcagaaccattttactttaaaattaataatattgtattagagaatatattaaatggttatattgggttatgttaggatatatacttgaatggaaatacatgtactattagcaatcatatttcatttatccctgtaattagacaagaaagcataatatagctctactcatgggtacacataccagtgtataagatttttagaagtttactttttaaaaataaaagcaaaatgtaagatcttaaaaaaaaaaaaaaaaaa

SEQ ID NO: 65 - protein, 793 aa, Homo sapiens; identical to SEQ ID NO: 17

SEQ ID NO: 66 - DNA, 1678 bp, Homo sapiens
ggaagtgctttctttgcccgccgttcgccaaacgaagtcgtggaggtggcgaaacgaggaggagataacgcggccttgggctctggtgtctcccagtctgctaaagccctaaggccatcaccatggacttccagcatcgccccgggggcaagaccgggagcgggggcgtggcctcctcctccgagagcaaccgtgaccgcagggagcgcctccggcagctggccctggagaccatcgacatcaacaaggacccgtacttcatgaagaaccacctgggctcctatgaatgcaaactctgcctgacacttcacaacaatgaggggagctacctggcacatacgcaggggaagaagcaccagaccaacctggcccggcgagcagccaaggaggccaaggaggcccctgcccagcccgcgcctgagaaggtcaaggtggaggtgaagaagtttgtgaagatcggccgcccgggctacaaagtgaccaagcagagagactcggagatgggccagcagagcctcctcttccagattgactaccctgagatcgccgagggcatcatgccacgtcaccgcttcatgtctgcgtacgagcagaggatcgagcctccggaccggcgctggcagtacctgctcatggccgccgagccctacgagaccattgccttcaaggtgccgagcagagagatcgacaaggcggagggcaagttctggacacactggaaccgggagaccaagcagttcttcctccagttccactttaagatggagaagcccccggctccacccagcctccctgctggcccccctggggtgaagcggcctccacccccgctgatgaacggtctgccccctcggccaccgctgcctgagtctttgccaccgcccccgccaggaggcctgcctctgccacccatgccccccacagggcctgcgccctcagggcccccgggaccaccccagctacccccgccagctccaggggtccaccccccggccccagtggtgcatccccctgcatctggggtccatcccccagctcctggcgtccaccccccagctcctggcgtccatcccccagcccctggggtccacccaccaacctctggggtccaccccccagctcctggagtccaccctccagcccccggggttcacccaccagcccccggagtccacccaccagcccctggggttcacccaccagccccaggggtccatcctcccccatcagcgggggttcacccccaggccccgggggtgcacccagcagcccccgccgttcaccctcaggccccaggggtgcacccaccagccccagggatgcaccctcaggccccgggggtccacccccaacctcccggggtccatccgtcggctcctggggtccaccctcagcctccgggagttcacccctcaaatcctggggtgcaccccccaactcccatgcccccaatgctgaggcccccacttccctccgaaggcccagggaacatacctccccctcccccaaccaactgagaagctgctccctcccccagcaagcccagcgccaggtgctcttgccttttcccactgagagaaggctgctcttttgtactgccccccgctcattaaacagcctcccccagccctgagtgcactgatgtccgcaaaaaaaaaaaaaaaaaaaaaaaaaaaaa

SEQ ID NO: 67 - protein, 464 aa, Homo sapiens
MDFQHRPGGKTGSGGVASSSESNRDRRERLRQLALETIDINKDPYFMKNHLGSYECKLCLTLHNNEGSYLAHTQGKKHQTNLARRAAKEAKEAPAQPAPEKVKVEVKKFVKIGRPGYKVTKQRDSEMGQQSLLFQIDYPEIAEGIMPRHRFMSAYEQRIEPPDRRWQYLLMAAEPYETIAFKVPSREIDKAEGKFWTHWNRETKQFFLQFHFKMEKPPAPPSLPAGPPGVKRPPPPLMNGLPPRPPLPESLPPPPPGGLPLPPMPPTGPAPSGPPGPPQLPPPAPGVHPPAPVVHPPASGVHPPAPGVHPPAPGVHPPAPGVHPPTSGVHPPAPGVHPPAPGVHPPAPGVHPPAPGVHPPAPGVHPPPSAGVHPQAPGVHPAAPAVHPQAPGVHPPAPGMHPQAPGVHPQPPGVHPSAPGVHPQPPGVHPSNPGVHPPTPMPPMLRPPLPSEGPGNIPPPPPTN

SEQ ID NO: 68 - DNA, 2855 bp, Homo sapiens
ttccggcactcgcggaactttggtgcagcctgatgcgcaacgtggggactcaggcgcgctgggcggcaggagttgcttccggccgtgttggtggtctgaattgagaagccgcgactaagggaagatggagacaatactggagcagcagcggcgctatcatgaggagaaggaacggctcatggacgtcatggctaaagagatgctcaccaagaagtccacgctccgggaccagatcaattctgatcaccgcactcgggccatgcaagataggtatatggaggtcagtgggaacctgagggatttgtatgatgataaggatggattacgaaaggaggagctcaatgccatttcaggacccaatgagtttgctgaattctataatagactcaagcaaataaaggaattccaccggaagcacccaaatgagatctgtgtgccaatgtcagtggaatttgaggaactcctgaaggctcgagagaatccaagtgaagaggcacaaaacttggtggagttcacagatgaagagggatatggtcgttatctcgatctccatgactgttacctcaagtacattaacctgaaggcatctgagaagctggattatatcacatacctgtccatctttgaccaattatttgacattcctaaagaaaggaagaatgcagagtataagagatacctagagatgctgcttgagtaccttcaggattacacagatagagtgaagcctctccaagatcagaatgaactttttgggaagattcaggctgagtttgagaagaaatgggagaatgggacctttcctggatggccgaaagagacaagcagtgccctgacccatgctggagcccatcttgacctctctgcattctcctcctgggaggagttggcttctctgggtttggacagattgaaatctgctctcttagctttaggcttgaaatgtggcgggaccctagaagagcgagcccagagactattcagtaccaaaggaaagtccctggagtcacttgatacctctttgtttgccaaaaatcccaagtcaaagggcaccaagcgagacactgaaaggaacaaagacattgcttttctagaagcccagatctatgaatatgtagagattctcggggaacagcgacatctcactcatgaaaatgtacagcgcaagcaagccaggacaggagaagagcgagaagaagaggaagaagagcagatcagtgagagtgagagtgaagatgaagagaacgagatcatttacaaccccaaaaacctgccacttggctgggatggcaaacctattccctactggctgtataagcttcatggcctaaatatcaactacaactgtgagatttgtggaaactacacctaccgagggcccaaagccttccagcgacactttgctgaatggcgtcatgctcatggcatgaggtgtttgggcatcccaaacactgctcactttgctaatgtgacacagattgaagatgctgtctccttgtgggccaaactgaaattgcagaaggcttcagaacgatggcagcctgacactgaggaagaatatgaagactcaagtgggaatgttgtgaataagaagacatacgaggatctgaaaagacaaggactgctctagtgttcagggatgtagctcagcttttgggctagcccaggcttccctaagatctgctttttctatttctcccaaccaaatcctcttaaagaccctttgctatgtagtctcatggtctagcatgcatcttgtagaaacaaggcatgctggcagattgcagggttgagatgtgttttatctgttttatattttaaaagattctgccagaaaataaaaccagaccttgttctaaagcccagggttatggaccaactcagtgcttcaggtcttaacgcctccatacctcttcctcaccaactttactagtagctgagatttaatgggcacctattatgctacatatcatgttaggtaaatctgacctgacctctttccccaccctcctttgttgctgcttccctgaatgagtattaccccaggatgaggtctgccatcagcttagttagccattgatgcaaatactagggaaagactaggaggatgagccagggttgctactaaggactaagtgtcgcaccaaggtttgccttttgtatttgcataaagaaaggagttggagctgggtgcagtggcttgtgcctgtagtcccagctacttgggaggctgaggcaggagggttgcttgagactagcctaggtaacatagtgagaccctgtctcattaaaaaaaaaaaaaaggcatggtggcacgcactgtagtcccagctactcaggagactgaggctagaagatcctttgaacctaggagtttgagaccagcctgggcgatatagtgaggccccatctcaaaaaaaaaaaaaggggggggggggggagttgggctgtgttggaatgggcctgcagcccaacaaacaagggaactaggaccgacagtgacttcaccagcttgctaggtcagaatgagagactggtgggtctgtctacctgtttcttctacaagatccctatttgactgtaaaagtagctaatactcacatgttctccaatcccaggtagccatggtagagttgggtagagttgagcagctgccccaggatccaaatgtggtgtctgaaatggaaagaactaaggcaaccaggaaggcactgatctgccttataagcacagtcatctgaaagtcaggcctgctgcaggacaggatcccccagagaccccatttgcctctcaacactcagaccttcaactgttttttaataaatctactttttaaaaaaaaaaaaaaaaaaaaa

SEQ ID NO: 69 - protein, 501 aa, Homo sapiens; identical to SEQ ID NO: 21

SEQ ID NO: 70 - DNA, 717 bp, Homo sapiens
atgtcagaggatttagcaaagcagctggcaagctacaaagctcagctccagcaagttgaagctgcattatctggaaatggagaaaatgaagatttgctaaaattgaagaaagatttacaagaagttatagaactaaccaaagaccttctgtcaactcaaccttctgagacgcttgcaagttcagacagttttgcttctactcaacctactcattcatggaaagtaggagacaagtgtatggcagtctggagtgaagatggacagtgttatgaagcggagattgaggagatagatgaagaaaatggcaccgctgcaatcacctttgctggttatggcaatgctgaagtgactccactgttgaacctcaagcctgtagaagaaggaaggaaggcaaaggaggacagtggcaacaaacccatgtcaaaaaaagaaatgattgcccagcagcgtgaatataaaaagaagaaagctttgaaaaaagctcagagaataaaagaacttgagcaggaaagagaggaccagaaagtgaaatggcaacaattcaacaacagagcctattctaaaaacaaaaaaggccaggtaaagaggagtatttttgcttcacctgagagtgtgactggtaaagttggagtaggaacctgtggaattgctgataaacctatgacacaatatcaagatacctctaaatacaatgtcaggcatttgatgcctcaataa

SEQ ID NO: 71 - protein, 238 aa, Homo sapiens
MSEDLAKQLASYKAQLQQVEAALSGNGENEDLLKLKKDLQEVIELTKDLLSTQPSETLASSDSFASTQPTHSWKVGDKCMAVWSEDGQCYEAEIEEIDEENGTAAITFAGYGNAEVTPLLNLKPVEEGRKAKEDSGNKPMSKKEMIAQQREYKKKKALKKAQRIKELEQEREDQKVKWQQFNNRAYSKNKKGQVKRSIFASPESVTGKVGVGTCGIADKPMTQYQDTSKYNVRHLMPQ

SEQ ID NO: 72 - DNA, 1153 bp, Homo sapiens
aactccagggctagtgagctggaccggaagtaggtttctacccgaccgcattttacgtggtgctgcatttccggtagcggcggcgggaaatcggctgtgggagagaggctaggcctctgaggaggcgaatccggcgggtatcagagccatcagaaccgccaccatgacggtgggcaagagcagcaagatgctgcagcatattgattacaggatgaggtgcatcctgcaggacggccggatcttcattggcaccttcaaggcttttgacaagcacatgaatttgatcctctgtgactgtgatgagttcagaaagatcaagccaaagaactccaaacaagcagaaagggaagagaagcgagtcctcggtctggtgctgctgcgaggggagaatctggtctcaatgacagtagagggacctcctcccaaagatactggtattgctcgagttccacttgctggagctgccgggggcccagggatcggcagggctgctggcagaggaatcccagctggggttcccatgccccaggctcctgcaggacttgctgggccagtccgtggggttggcgggccatcccaacaggtgatgaccccacaaggaagaggtactgttgcagccgctgcagctgctgccacagccagtattgccggggctccaacccagtacccacctggccgtgggggtcctcccccacctatgggccgaggagcaccccctccaggcatgatgggcccacctcctggtatgagacctcctatgggtcccccaatggggatcccccctggaagagggactccaatgggcatgccccctccgggaatgcggcctcctccccctgggatgcgaggccttctttgacccttggccacagagtatggaagtagctccgcagaggcgtgggctcgattcctcagggccacgttaccacagacctgtttgtttcttatgctgttgttcgtggagtctcatgggattgtctggtttcccttacagggccccctcccccgggaatgcgcccaccaaggccctagactcatcttggccctcctcagctccctgcctgtttcccgtaaggctgtacatagtccttttatctccttgtggcctatgaaactggtttataataaactcttaagagaacattataattgc

SEQ ID NO: 73 - protein, 231 aa, Homo sapiens
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGLL

SEQ ID NO: 74 - DNA, 1385 bp, Homo sapiens
gggaagtcctggcccttggcccaacctcctgctgtccccggtctgagggcccaagccccgcgtctccgccttgccgtccagcctgtccttggtgtggggtgcttggaagtgtgagcaccctctctggctctttgccggccccaagggtcgttgcggcggcccccgggcccagtcatcagccctcttttccggtgccggaactatcgtactggtgccctagtttcccagccggcaggcggagtgaggaggaagcggaggcgagaccgcgctcaacactggggaaaggaaggaggcgggccctgcgcgcagcattttgggaaagagtggaattctgggtgttaggcccgccattcgcttgactcacgccttcgccgtagcatctttcgcagcggaccgaagagaagaaaagtaggccagagccgaactctcttcctgccaagatgtctattggtgtgccgattaaagtactgcatgaggccgagggccacattgtgacatgtgagacgaacaccggtgaggtatatcgggggaagctcattgaagcagaggacaacatgaactgccagatgtccaacatcacagtcacatacagagatggccgagtggcacagctggagcaggtatacatccgtggcagcaaaatccgctttctgattttgcctgacatgctgaagaacgcacccatgttaaagagcatgaaaaataaaaaccaaggctcaggggctggccgaggaaaagctgctattctcaaggcccaagtggccgcaagaggaagaggacgtggaatgggacgtggaaacatctttcaaaagcgaagataattttctaagttgaacagaactttgtccttttttctttcaggttatctgagttcattggagtgggtgcttgtgcatatatgctaggtatcttttgccatctttctctttagatcaggggaaatgtttaagctaaataaatctggggggttttttgttctgttttgttttgttttctttgagaaaataaggactttgtgttcatttgaagtttgctttggctaaattttgacaccctgggggatgtcctgggagaatgccagtactttgaaatagcacagctattgatgagattttaagctgctgttcctggccagttgcaggttaagccccagaaaagttcaccttggagaagctccgagaacacacagattgtgtctcattcatcttcagacacaggcacatagtgtggcactttgttcagttaacatttgtggagctgtcatcagctcagctttaacaacacaggtgactctttcccttgataaagtcataggtaattcaggggctgggtgcggcggctcacgcatgtaatcccagcactttgggaggccaaggcgggcggat

SEQ ID NO: 75 - protein, 126 aa, Homo sapiens
MSIGVPIKVLHEAEGHIVTCETNTGEVYRGKLIEAEDNMNCQMSNITVTYRDGRVAQLEQVYIRGSKIRFLILPDMLKNAPMLKSMKNKNQGSGAGRGKAAILKAQVAARGRGRGMGRGNIFQKRR

SEQ ID NO: 76 - DNA, 762 bp, Homo sapiens
taccattcttcaagaaacggtttgaatcagactgcctttccttttgtcttcattgtcataaacatctgcccccgtgtggttctgactggccgcgaacccctacccgaagcttttattccatcattgtgcaccgttggtggggaatgctgtggcaacaggccacgcctccacttactggttggctttgcgcaggcgccaacggaagtgggtcgcaggaagaggaagtcccgcctctctctcctcaggcagcagcaacgcggaggaaacgggagtgaacggagagcgtagtgaccatcatgagcctcctcaacaagcccaagagtgagatgaccccagaggagctgcagaagcgagaggaggaggaatttaacaccggtccactctctgtgctcacacagtcagtcaagaacaatacccaagtgctcatcaactgccgcaacaataagaaactcctgggccgcgtgaaggccttcgataggcactgcaacatggtgctggagaacgtgaaggagatgtggactgaggtacccaagagtggcaagggcaagaagaagtccaagccagtcaacaaagaccgctacatctccaagatgttcctgcgcggggactcagtcatcgtggtcctgcggaacccgctcatcgccggcaagtaggggccgcctgtctgttgacagaactcactcctctgtcctatgaagaccgctgccattggtgttgagaataataaagctctgtgtttttttctaaaaaaaaaaaaaaaaa

SEQ ID NO: 77 - protein, 118 aa, Homo sapiens
MSLLNKPKSEMTPEELQKREEEEFNTGPLSVLTQSVKNNTQVLINCRNNKKLLGRVKAFDRHCNMVLENVKEMWTEVPKSGKGKKKSKPVNKDRYISKMFLRGDSVIVVLRNPLIAGK

SEQ ID NO: 78 - DNA, 1614 bp, Homo sapiens
agtgctccgcgcgctcttgacgtccggagcccctggagtaggcgcttccggccattcatactgcagtcggtcagtgttcggttgaaggattctgtgtgctgtcggacccagagggtgacggcgccgctaggatgaagctcgtgagatttttgatgaaattgagtcatgaaactgtaaccattgaattgaagaacggaacacaggtccatggaacaatcacaggtgtggatgtcagcatgaatacacatcttaaagctgtgaaaatgaccctgaagaacagagaacctgtacagctggaaacgctgagtattcgaggaaataacattcggtattttattctaccagacagtttacctctggatacactacttgtggatgttgaacctaaggtgaaatctaagaaaagggaagctgttgcaggaagaggcagaggaagaggaagaggaagaggacgtggccgtggcagaggaagagggggtcctaggcgataatgtctctcaagatttcaaagtcatatgagatttgggatattttttgtacaggttgtgtttgtttatgtcagtttttaataaacataaatgtgggacagagctgtctatttagtatatcaaagttttagtagtttcctccacattcacgaaattaccacagtgagagctaagcatttctactgggcagtttcatttttagttgatcaggttttaagtttttgaactaaaatttttctttttctttttatgatgaataaggttaaaataaaagccttagacaaattaaatttggcagagtttaattgagcaaaggacaattcacaaatcaggtagcccctgaaccataataggctcagaggcttcagcccagctgcatagttgaagatttatggacagaaggaaagtgatgtatggaaaatggaagtgagatacagcaacagccggattagttacagttcagcgtttgccttatttgaatatggtttgaacagttcgctgtctttggttggctgaaacttagtgattgccacaagagtagggtaccgtctgtttacacgtccagttaggctacagttctatgtactgagaaacctttaagctgaacttgagatatgtaaagagactttaggctaaacttaacaatatatataggatatatacccttctacttcacatgcactgaatatgcattttattgctttactcttcattctgtggcacctacccacaggggaagtaagaagtttgttttggtatttcggaaactaaagtccttatgggatggggtctagaattgattctcctttcctgagttttactccacggagtcttaggtacctggtaaaaagttgtcttctaaattaagggtcattgctttgttgtctagctgctaatgtcttacttttgtttcttttgctttttaatcagttcttaataggatatagttttatgttttccaagttataacttggagttaatggtcactagattatcagttatgagcagtgttaaaatctcctattaatgtgtaatgtacctgtcagtgcctcctttattaaggggttctttgagaataaaagagaaaagacctactttatttgacagcaaaaaaaaaaaggaattc

SEQ ID NO: 79 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 80 - DNA, 1593 bp, Homo sapiens
gttgctctcagaggcagcgtgcgggtgtgctctttgtgaaattccaccatggcgtaccgtggccagggtcagaaagtgcagaaggttatggtgcagcccatcaacctcatcttcagatacttacaaaatagatcgcggattcaggtgtggctctatgagcaagtgaatatgcggatagaaggctgtatcattggttttgatgagtatatgaaccttgtattagatgatgcagaagagattcattctaaaacaaagtcaagaaaacaactgggtcggatcatgctaaaaggagataatattactctgctacaaagtgtctccaactagaaatgatcaatgaagtgagaaattgttgagaaggatacagtttgtttttagatgtcctttgtccaatgtgaacatttattcatattgttttgattaccctcgtgttactacaagatggcaataaatactatgggattgtttgtattaaaaaatttacattgcttcttactattcagcagtagaaactttttacacagtaacaccattcgttgttggtatttagttttctgaagggtcgcagttgccttgagcacttggtattcgcagagcttggacctgtagattttgaggcagattaggaattctgcctgatgggtaagcttccagtattgggaggtggagaaggggagggttcagaaaaataaataagagttattgcactaacaaaagtcttcatcacttgtagttctggatgctggaataccagagtttctaacctaaatacgttgggtacattatttaatggggtcagtattgctcaacactctcattgagtcactgtgaggtctttgtgaattttatcgctaagatcagaatgtgagaagtatttggatatagggaaagaatgaagtgcctttcaagtacattaaaaatcagttaagagtttacaggaaagagactgagattggtaatctgaaaaggaccacaagcatagctggtaggcaaattgttaggcagaataatggtatgttggagaaatacacctgtctcttattcttatctcttaagaattttggttcttcccccagtgattgctggttgaattcttatatggacaggtttcccttcccccagtatcgcacatggttctagttagaatcctgttagatagtgagcatctgctgttagctagacactgttgaatcaatacagtgaaacagacaggtaagcaggtggttttaatgcctcattctaagtgctatttaaattggatcttgaaagatgaataaggcctgccttaagttctgctaaaggcaatgttgttattaaggacgggtgaggaaggaccaagaagtctggctcctgcttttctacatgcctgtgaaggagacttttcatgaaactgaataaaagaaatcctttccgagatagaggagatttgtaggtgaatgcagaagtgtatccagctttgaaccatgcattccatctatattcctcctgtctgaactcatgctcttttgattatgttttgaaataaagggctgcattagttcttttaaaaaaaaaaaaaaaaaaaaaaaaaaaaaaaaaaaaaaaaaaaaaaaa

SEQ ID NO: 81 - protein, 92 aa, Homo sapiens
MAYRGQGQKVQKVMVQPINLIFRYLQNRSRIQVWLYEQVNMRIEGCIIGFDEYMNLVLDDAEEIHSKTKSRKQLGRIMLKGDNITLLQSVSN

SEQ ID NO: 82 - DNA, 462 bp, Homo sapiens
tctggccatttctcttgaaactgcggctcgggacctgcggtacctgctgtagtcacgagggacgggcggcggctggtcggcagagagtagcctgcaacattcggccgtggtttacatgagtttacccctcaatcccaaacctttcctcaatggactaacaggaaagccagtgatggtgaaacttaagtggggaatggagtacaagggctatctggtatctgtagatggctacatgaacatgcagcttgcaaatacagaagaatacatagatggagctttgtctggacatctgggtgaagttttaataaggtgtaataatgtcctttatatcagaggtgtggaagaagaggaagaagatggggaaatgagagaatagcatcttttgtgggggattttttttatatatatttctagacaataaagatttgtttgtttttcaacttgaaaaaaaaaaaaaaaaaa

SEQ ID NO: 83 - protein, 86 aa, Homo sapiens
MSLPLNPKPFLNGLTGKPVMVKLKWGMEYKGYLVSVDGYMNMQLANTEEYIDGALSGHLGEVLIRCNNVLYIRGVEEEEEDGEMRE

SEQ ID NO: 84 - DNA, 606 bp, Homo sapiens
agacgcaagacgccgggcctacagcgggagcgtgaggaaagccgtgcgttgcgttccaaggcatctgtgagcccgcggagtatacaccatgagcaaagctcaccctcccgagttgaaaaaatttatggacaagaagttatcattgaaattaaatggtggcagacatgtccaaggaatattgcggggatttgatccctttatgaaccttgtgatagatgaatgtgtggagatggcgactagtggacaacagaacaatattggaatggtggtaatacgaggaaatagtatcatcatgttagaagccttggaacgagtataaataatggctgttcagcagagaaacccatgtcctctctccatagggcctgttttactatgatgtaaaaattaggtcatgtacattttcatattagactttttgttaaataaacttttgtaatagtcaaaaatgctttctcagatgttctgaatatagaatatcagctctcattccagttttttctaacatgaattttcctggttgacattgatttcaaagggttttatgcattaaagtgaaagaatcttattaaatgcgaaaaaaaaaaaaaaaaaaaaaaaaaaaaa

SEQ ID NO: 85 - protein, 76 aa, Homo sapiens
MSKAHPPELKKFMDKKLSLKLNGGRHVQGILRGFDPFMNLVIDECVEMATSGQQNNIGMVVIRGNSIIMLEALERV

SEQ ID NO: 86 - DNA, 21 bp, Artificial Sequence
  Description of Artificial Sequence Synthetic PCR primer U2 Foward
agtgtagtatctgttcatcag

SEQ ID NO: 87 - DNA, 21 bp, Artificial Sequence
  Description of Artificial Sequence Synthetic PCR U2 Reverse
atctccctgctccaaaaatcc

SEQ ID NO: 88 - DNA, 23 bp, Artificial Sequence
  Description of Artificial Sequence Synthetic anti-sense oligonucleotide
ataagaacagatactacacttga

(19) United States
(12) Patent Application Publication
(10) Pub. No.: US 2006/0068434 A1
(43) Pub. Date: Mar. 30, 2006

(54) Methods and compositions for detecting cancer using components of the U2 spliceosomal particle

(76) Inventor: Jay Stoerker, Framingham, MA (US)

Correspondence Address: GOODWIN PROCTER LLP;PATENT ADMINISTRATOR, EXCHANGE PLACE, BOSTON, MA, 02109-2881, US

(21) Appl. No.: 11/232,440

(22) Filed: Sep. 21, 2005

Related U.S. Application Data
(60) Provisional application No. 60/612,310, filed on Sep. 21, 2004.

(51) Int. Cl. C12Q 1/68; G01N 33/574

(52) U.S. Cl. 435/6; 435/7.23

(57) ABSTRACT

The present invention relates to cancer-associated proteins and nucleic acids that encode or bind specifically to cancer-associated proteins, which represent markers for cancer detection. Specifically, the invention provides a family of methods and compositions for detecting cancer, for example, breast cancer, in an individual using components of the U2 spliceosomal particle. A target cancer-associated protein may be detected, for example, by reacting the sample with a labeled binding moiety, for example, a labeled antibody capable of binding specifically to the protein. The invention also provides kits useful in the detection of cancer in an individual.

### REFERENCE TO RELATED APPLICATIONS

This application claims the benefit of U.S. Patent Application Ser. No. 60/612,310, filed Sep. 21, 2004, the disclosure of which is incorporated by reference herein.

### FIELD OF THE INVENTION

The present invention relates generally to methods and compositions for the detection and/or treatment of cancer. More specifically, the present invention relates to cancer-associated proteins and nucleic acids that encode or bind specifically to such cancer-associated proteins, which represent markers for cancer detection.

### BACKGROUND OF THE INVENTION

Breast cancer is one of the leading causes of death in women. While the pathogenesis of breast cancer is unclear, transformation of normal breast epithelium to a malignant phenotype may be the result of genetic factors, especially in women under 30 years of age (Miki et al. (1994) Science 266: 66-71). However, it is likely that other, non-genetic and epigenetic factors also have a significant effect on the etiology of the disease and its natural history. Regardless of the cancer's origin, cancer morbidity increases significantly if it is not detected early in its progression. Because of the premium placed on early detection in the management of cancer, great medical and commercial effort has been focused in the last three decades on meeting this goal. For example, research has linked alleles of the BRCA1 and BRCA2 genes to hereditary and early-onset breast cancer (Wooster et al. (1994) Science 265: 2088-2090). However, it is understood that BRCA mutations fail to account for the majority of breast cancers (Ford et al. (1995) British J. Cancer 72: 805-812).

There is, therefore, a need in the art for specific, reliable markers that are differentially expressed in normal and cancerous tissue and that may be useful in the diagnosis of cancer, in the prediction of its onset, or the treatment of cancer. Such markers and methods for their use are provided herein.

### SUMMARY OF THE INVENTION

The invention provides a variety of methods and compositions for detecting the presence of cancer, for example, breast cancer, in a human or other mammal. The invention is based, in part, upon the discovery that components of spliceosomal particle U2, also referred to herein as the U2 particle, are detectable at a higher concentration in a sample (for example, a body fluid) harvested from a mammal with cancer relative to a corresponding sample from a normal mammal, that is, a mammal without the cancer. The invention also is based, in part, upon the discovery that these components can be observed in an intact complex present in a sample, for example, a body fluid sample, from a mammal with cancer. Accordingly, the complex and its components can be used as cancer markers useful in diagnosing or monitoring the status of a cancer.

The components of the U2 particle include a small nuclear RNA called “U2 snRNA” and a plurality of different proteins. The protein components include, for example, U2 snRNP B″, SAP155, SAP145, SPF31, SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30. The protein components also include Sm proteins, such as, SmB/B′, SmD3, SmD2, SmD1, SmE, SmF, and SmG. It is understood that certain of the Sm proteins are also found in spliceosomal particles other than the U2 particle.

The invention provides methods for detecting or monitoring a cancer in a mammal by detecting the presence, absence, or amount (which can be an absolute amount or a relative amount) of one or more components of the U2 particle. The methods of the invention may be performed on any relevant tissue or body fluid sample. For example, methods of the invention may be performed on breast tissue, such as breast biopsy tissue. Alternatively, the methods of the invention may be performed on a human body fluid sample such as blood, serum, plasma, nipple aspirate, ductal lavage fluid, fine needle aspirate, sweat, tears, urine, peritoneal fluid, lymph, vaginal secretions, semen, spinal fluid, ascitic fluid, saliva or sputum. It is contemplated, however, that the methods of the invention also may be useful in detecting cancer in other tissue or body fluid samples.

Detection of cancer can be accomplished using any one of a number of assay methods well known and used in the art. For example, a protein or nucleic acid can be detected by a spectroscopic approach such as mass spectrometry or fluorescence spectroscopy, or through the use of a binding moiety that specifically binds the protein or nucleic acid, as in an immunoassay, a nucleic acid hybridization method, or a method such as RT-PCR involving amplification of a nucleic acid.

Certain methods for detecting breast cancer by detecting U2 snRNP B″ are known in the art. See, for example, U.S. Pat. No. 6,936,424. The present invention, however, provides improved methods of detecting U2 snRNP B″ and other components of the U2 particle, based in part upon the discovery that these components are present as an intact complex in a body fluid in mammals with cancer. Accordingly, in one aspect, the invention relates to a method of diagnosing cancer in a mammal by disrupting a complex comprising one or more components of the U2 particle prior to detecting and/or measuring the amount of one or more of the components. The component can be U2 snRNP B″, U2 snRNA, or another component of the U2 particle, such as SAP155, SAP145, SPF31, SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30.

In certain embodiments, disruption of the complex, which can be achieved by providing a chemical denaturant, heat, an acid, a base, a salt, or another factor known to affect protein-protein or protein-nucleic acid interactions, facilitates the subsequent detection and/or measurement of the U2 particle component. For example, if a U2 particle component is subsequently detected using a binding moiety that specifically binds the component, disruption of the complex can increase the accessibility of the component to a binding moiety. Alternatively, if a U2 particle component is subsequently detected by mass spectrometry, disrupting the complex in advance can simplify the mass spectrometry analysis.

In one embodiment, the presence of the component is indicative of the presence of cancer, for example, breast cancer, in a mammal. In another embodiment, an amount of the component is indicative of the presence of cancer, for example, breast cancer, in a mammal.

In another aspect, the invention provides methods that relate to combining a tissue or body fluid sample isolated from a mammal with a purified binding moiety with an affinity for a component of the U2 particle other than U2 snRNP B″ to form a complex. The methods further involve detecting and/or measuring the amount of the complex of the component. It is important to note that these methods using one or more non-B″ components of the U2 particle may advantageously be combined with detection of U2 snRNP B″ or with use of a binding moiety with an affinity for U2 snRNP B″. For example, an anti-U2 snRNP B″ antibody can be used to purify a U2 particle prior to detection of U2 snRNA or of another protein component of the particle. As another example, an antibody that binds specifically to the 2,2,7-trimethylguanosine cap of the U2 snRNA molecule can be used to purify the U2 particle prior to detection of U2 snRNP B″, for example, by immunoassay or mass spectrometry.

In one embodiment, the method of detecting or monitoring a cancer, for example, breast cancer, includes exposing a sample isolated from the mammal to a purified binding moiety capable of binding specifically to a component of spliceosomal particle U2. The binding moiety forms a complex with the component; the presence, absence or amount of the complex is then detected or determined, providing information indicative of the presence or absence of the cancer in the mammal.

The binding moiety can be a protein with an affinity for the snRNA, such as a snuportin protein, or for a protein component of the spliceosomal particle other than U2 snRNP B″, such as SAP155, SAP145, SPF31, SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30. For example, the binding moiety can be an antibody or an antigen-binding fragment thereof. Exemplary antibodies include, for example, an anti-2,2,7-trimethylguanosine antibody, an anti-Sm antibody, an anti-SMN antibody, an anti-Importin B antibody, an anti-snuportin antibody, an anti-Ran antibody, or an anti-Ran-GTP antibody. The binding moiety alternatively can be a nucleic acid or a nucleic acid analog (such as a peptide nucleic acid, a locked nucleic acid, or other nucleic acid analog, for example, a morpholino containing oligonucleotide) having an affinity for the U2 snRNA or for a protein component of the spliceosomal particle.

After complex formation, the presence, absence, or amount of the complex can be determined by mass spectrometry, RT-PCR, immunoassay or use of another labeled or unlabeled binding moiety, or another assay technique known in the art. The detecting and/or measuring step can involve detection of a second, different component of the U2 particle, which could be any component, including U2 snRNA, U2 snRNP B″, or another protein component.

The invention also is based, in part, upon the discovery that a binding moiety that specifically binds 2,2,7-trimethylguanosine can be used to purify a naturally-occurring, circulating snRNA bearing a 2,2,7-trimethylguanosine cap, even in the complex environment of a mammalian body fluid. Accordingly, in one aspect, the invention provides a method of detecting one or more snRNAs bearing a 2,2,7-trimethylguanosine moiety by contacting the sample with a binding moiety, such as a snuportin protein, an antibody, or an antigen-binding fragment thereof, that specifically binds 2,2,7-trimethylguanosine to permit complex formation between the binding moiety and the one or more snRNAs and detecting the presence or absence of the complex. The presence or amount of complex formation can be indicative of the presence or extent of cancer in the mammal.

In another aspect, the invention provides kits for purifying or detecting a U2 snRNA. In one embodiment, the kit includes (i) a purified binding moiety that specifically binds 2,2,7-trimethylguanosine and (ii) one or more molecules (nucleic acids or nucleic acid analogs) complementary to at least a portion of a U2 snRNA. Generally, the molecules are complementary to a portion at least three nucleotides in length, and preferably at least five, at least eight, or at least ten nucleotides in length. The purified binding moiety can be a snurportin protein or an antibody or antigen-binding fragment thereof.

In another embodiment, the kit includes a purified binding moiety that specifically binds a U2 snRNA and one or more reference samples having amounts of U2 snRNA indicative of the presence of a cancer such as breast cancer. Thus, for example, a tissue or body fluid sample from a mammal with the cancer can be provided as a positive control. Alternatively, a synthetic U2 snRNA sequence or a fragment thereof can be provided as a positive control. The binding moiety can be, for example, a nucleic acid or nucleic acid analog complementary to at least a portion of the U2 snRNA; an antibody or antigen-binding fragment thereof; or a snuportin protein. The kit also optionally includes a receptacle for receiving a sample from a patient.

Thus, the invention provides a family of methods and compositions for detecting and monitoring the status of a cancer in a mammal, such as a human. Specifically, the invention provides improved methods for detecting and/or measuring cancer marker U2 snRNP B″. The invention also provides methods for detecting and/or measuring other cancer markers present in the U2 particle. The invention provides methods for breast cancer-associated proteins, which permit specific and early, preferably before metastases occur, detection of breast cancer in an individual. In addition, the invention provides kits useful in the detection of breast cancer in an individual. In addition, the invention provides methods utilizing the breast cancer-associated proteins as targets and indicators, for treating breast cancers and for monitoring of the efficacy of such a treatment. These and other numerous additional aspects and advantages of the invention will become apparent upon consideration of the following figures, detailed description, and claims which follow.

### DESCRIPTION OF THE DRAWINGS

The invention can be more completely understood with reference to the following drawings, in which:

FIG. 1 is a schematic representation showing the involvement of the U2 spliceosomal particle in the removal of an intron during mRNA maturation;

FIG. 2 is a schematic representation showing the relative positioning of several components of the U2 particle;

FIG. 3 is a schematic representation showing the structure of mature U2 snRNA as described in Lührmann et al. (1990) Biochim. Biophys Acta 1087: 265-292;

FIG. 4A is a schematic representation of a morpholine residue and FIG. 4B is schematic representation of a short segment of a morpholino containing oligonucleotide comprising two subunits joined by an intersubunit linkage; and

FIG. 5 is a picture of a gel showing the presence of U2 snRNA amplified in greater amounts from five serum samples from women diagnosed with breast cancer (denoted “C”) than the amounts from three serum samples from healthy women (denoted “N”).

### DETAILED DESCRIPTION OF THE INVENTION

The present invention provides methods and compositions for the detection of snRNAs, spliceosomal particle U2 and components thereof, and cancer. The invention is based, in part, upon the discovery that components of the U2 particle are present in a complex in a tissue or body fluid from mammals with cancer and that the components are detectable at a higher concentration in samples from mammals with a cancer than in samples from healthy mammals. It is understood that the term cancer embraces both cancerous lesions and pre-cancerous lesions. Although U2 snRNA and components of the U2 particle are known in the art, it was not previously appreciated that levels of U2 snRNA or, indeed, of circulating complexes containing known components of the U2 particle, can differentiate a tissue or body fluid of a healthy individual from a tissue or body fluid of an individual with cancer.

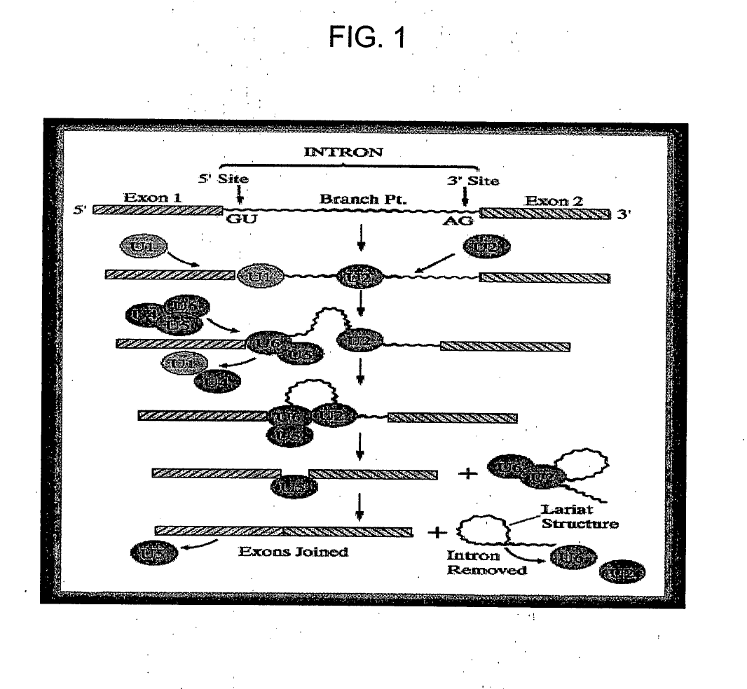

The U2 particle is involved in the maturation of messenger RNA as depicted in FIG. 1. The top line of FIG. 1 shows a portion of an immature or heterologous RNA. Two exons (exon 1 and exon 2), which ultimately are united in the mature message, are separated by an intervening intron. The intron contains within it a “branch point” sequence, which is used to guide the maturation process. Without wishing to be bound by theory, it is understood that the U1 particle binds to the upstream consensus sequence (GU), while at the same time, the U2 particle binds to the branch point. The U2 particle remains bound to the branch point, while the U1 is displaced by the U4/5/6 complex at the GU site. The U2 particle then mediates the connection of U5 between the exon units, while at the same time liberating U4 and U6 in turn. U2 and U6 form a meta-stable “lariat” structure with the intronic RNA. Ultimately, all the U particles involved in this splicing event are released and recycled in the cell.

It has been found that components of the U2 particle are detectable in a circulating body fluid in individuals with cancer and that one or more of the components are present in a complex in the body fluid. Without wishing to be bound by theory, it is contemplated that snRNPs normally buried within the nucleus may be externalized by apoptosis or oxidative stress associated with cancer. These externalized snRNPs find their way into the serum where they remain largely intact for a period of time. The present invention takes advantage of the relative stability of the complex in body fluids and the tissues contacted by those fluids to permit detection of the cancer even using a sample taken at a location potentially remote from the site of the cancer.

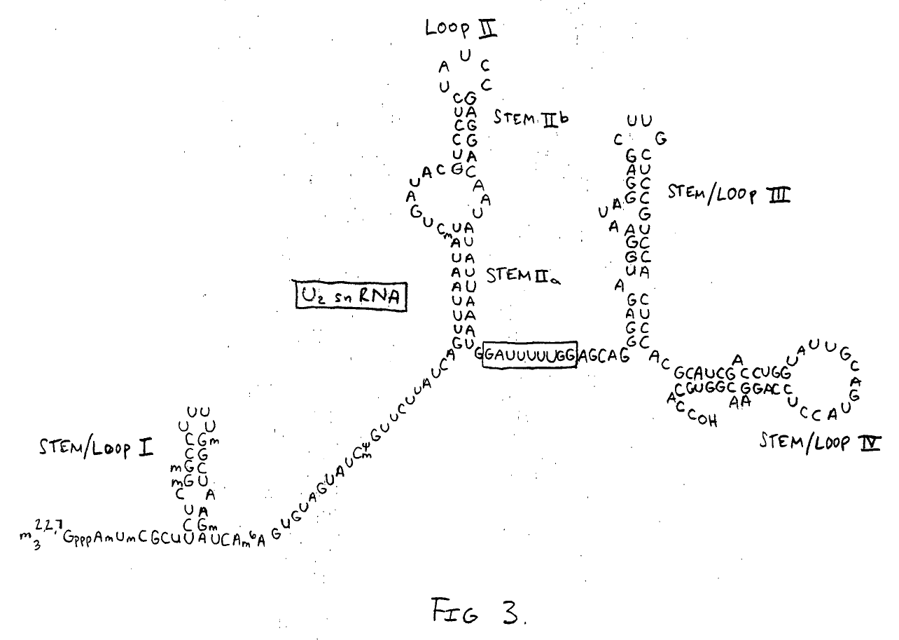

The U2 particle includes the U2 snRNA and a plurality of different proteins. The sequence of a human gene encoding U2 snRNA is set forth in SEQ ID NO. 1. The RNA of U2 snRNA sequence appearing in FIG. 3 corresponds substantially to residues 259-446 in SEQ ID NO. 1. It appears that the thymidine residue appearing as residue “299” of SEQ ID NO. 1 is replaced by a bond in FIG. 3. It is contemplated that this and other differences may result from naturally occurring variants of mammalian U2 snRNA. Accordingly, the term U2 snRNA includes RNA molecules having at least 80%, optionally at least 85%, and, optionally at least 90% identity to residues 259-446 of SEQ ID NO. 1 or fragments thereof containing 40 contiguous bases.

In order to determine the percentage identity of a test sequence relative to a reference nucleotide sequence, the candidate sequence and the reference sequence can be compared using the BLAST 2 SEQUENCE program (which produces the alignment of two given sequences using the BLAST engine for local alignment) using all the default parameters. This software is available from the National Center for Biotechnology Information (“NCBI”).

The proteins in the U2 particle include, for example, U2 snRNP B″, SAP155, SAP145, SPF31, SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30. The protein components also include Sm proteins, such as, SmB/B′, SmD3, SmD2, SmD1, SmE, SmF, and SmG. It is understood that certain of the Sm proteins are also found in spliceosomal particles other than the U2 particle. Of these, at least the thirteen proteins, SAP155, SAP145, SAP130, SAP114, SAP62, SAP61, SAP49, U2 snRNP A1, U2 snRNP B″, p14, U2AF35, U2AF65, and U2AF 1-RS2, are specific to the U2 particle. The U2 particle can be isolated in a 12S and a 17S form (Behrens, S. E. et al. (1993) Proc. Natl. Acad. Sci. USA 90:8229-33; Behrens, S. E. et al. (1993) Mol. Cell Biol. 13:307-19; and Hartmuth, K. et al. (2002) Proc. Natl. Acad. Sci. USA 99:16719-16724). The B″ protein is a stable component of both forms (Brehrens, S. E. et al. (1993) Proc. Natl. Acad. Sci. USA 90:8229-33). In contrast, the two essential multimeric splicing factors, SF3a and SF3b, are present only in the 17S form (Behrens, S. E. et al. (1993) Proc. Natl. Acad. Sci. USA 90:8229-33); Behrens, S. E. et al. (1993) Mol. Cell Biol. 13:307-19; Brosi, R. et al. (1993) Science 262:102-05; Kramer, A. et al. (1999) J. Cell. Biol. 145:1355-68; Staknis, D. et al. (1994) Mol. Cell Biol. 14). SF3a consists of three subunits (spliceosome-associated proteins (SAPs) 61, 62 and 114), and SF3b consists of four subunits (SAPs 49, 130, 145 and 155) (Brosi, R. et al. (1993) J. Biol. Chem. 268:17640-46; Das, B. K. et al. (1999) Mol. Cell Biol. 19:6796-802; Kramer, A. et al. (1999) J. Cell Biol. 145:1355-68).

U2 snRNP B″ includes a protein having the amino acid sequence set forth in SEQ ID NO. 3 and variants thereof. Variants include (a) a protein having at least 85% sequence identity, more preferably at least 90% sequence identity to the amino acid sequence set forth in SEQ ID NO. 3, (b) a protein having an amino acid sequence comprising the consensus sequence set forth in SEQ ID NO. 4, wherein Xaa represents any amino acid, or (c) a protein fragment comprising at least 25 consecutive amino acids set forth in SEQ ID NO. 3 or SEQ ID NO. 4. The fragments optionally have at least 75%, optionally at least 85%, and optionally at least 90% of the biological activity of the full length protein set forth in SEQ ID NO. 3. It is understood that the variants include allelic variants of U2 snRNP B″. Furthermore it is understood that U2 snRNP B″ includes a protein that binds specifically to an antibody that binds specifically to the protein of SEQ ID NO. 3. A gene encoding the U2 snRNP B″ protein of SEQ ID NO. 3 is set forth in SEQ ID NO. 2.

SAP155 includes a protein having the amino acid sequence set forth in SEQ ID NO. 6 and variants thereof. Variants include (a) a protein having at least 85% sequence identity, more preferably at least 90% sequence identity to the amino acid sequence set forth in SEQ ID NO. 6, (b) a protein having an amino acid sequence comprising the consensus sequence set forth in SEQ ID NO. 7, wherein Xaa represents any amino acid, or (c) a protein fragment comprising at least 25 consecutive amino acids set forth in SEQ ID NO. 6 or SEQ ID NO. 7. The fragments optionally have at least 75%, optionally at least 85%, and optionally at least 90% of the biological activity of the full length protein set forth in SEQ ID NO. 6. It is understood that the variants include allelic variants of SAP155. Furthermore it is understood that SAP155 includes a protein that binds specifically to an antibody that binds specifically to the protein of SEQ ID NO. 6. A gene encoding the SAP155 protein of SEQ ID NO. 6 is set forth in SEQ ID NO. 5.

SAP145 includes a protein having the amino acid sequence set forth in SEQ ID NO. 9 and variants thereof. Variants include (a) a protein having at least 85% sequence identity, more preferably at least 90% sequence identity to the amino acid sequence set forth in SEQ ID NO. 9, (b) a protein having an amino acid sequence comprising the consensus sequence set forth in SEQ ID NO. 10, wherein Xaa represents any amino acid, or (c) a protein fragment comprising at least 15 consecutive amino acids set forth in SEQ ID NO. 9 or SEQ ID NO. 10. The fragments optionally have at least 75%, optionally at least 85%, and optionally at least 90% of the biological activity of the full length protein set forth in SEQ ID NO. 9. It is understood that the variants include allelic variants of SAP145. Furthermore it is understood that SAP 145 includes a protein that binds specifically to an antibody that binds specifically to the protein of SEQ ID NO. 9. A gene encoding the SAP 145 protein of SEQ ID NO.9 is set forth in SEQ ID NO. 8.

SPF31 includes a protein having the amino acid sequence set forth in SEQ ID NO. 12 and variants thereof. Variants include (a) a protein having at least 85% sequence identity, more preferably at least 90% sequence identity to the amino acid sequence set forth in SEQ ID NO. 12, (b) a protein having an amino acid sequence comprising the consensus sequence set forth in SEQ ID NO. 13, wherein Xaa represents any amino acid, or (c) a protein fragment comprising at least 25 consecutive amino acids set forth in SEQ ID NO. 12 or SEQ ID NO. 13. The fragments optionally have at least 75%, optionally at least 85%, and optionally at least 90% of the biological activity of the full length protein set forth in SEQ ID NO. 12. It is understood that the variants include allelic variants of SPF31. Furthermore it is understood that SPF31 includes a protein that binds specifically to an antibody that binds specifically to the protein of SEQ ID NO. 12. A gene encoding the SPF31 protein of SEQ ID NO. 12 is set forth in SEQ ID NO. 11.

Sequence similarity searches using the BLAST algorithm were conducted in the NCBI's GenBank database using SAP155 (SEQ ID NO. 6), SAP145 (SEQ ID NO. 9), U2 snRNP B″ (SEQ ID NO. 3), and SPF31 (SEQ ID NO. 12) proteins as queries. The BLAST results were filtered to identify sequences for mammalian proteins that had an average identity greater than 85% and that had a total length of all BLAST HSP greater than 90% of the query length. Multiple sequence alignments were produced using CLUSTAL W (1.82). All parameters were set as the default. Consensus sequences were constructed based on manual analysis of multiple alignments. In the consensus sequences, “Xaa” represents an alternate amino acid residue or a peptide bond.

SAP130 includes a protein having the amino acid sequence set forth in SEQ ID NO. 15 and variants thereof. Variants include (a) a protein having at least 85% sequence identity, more preferably at least 90% sequence identity to the amino acid sequence set forth in SEQ ID NO. 15, or (b) a protein fragment comprising at least 25 consecutive amino acids set forth in SEQ ID NO. 15. The fragments optionally have at least 75%, optionally at least 85%, and optionally at least 90% of the biological activity of the full length protein set forth in SEQ ID NO. 15. It is understood that the variants include allelic variants of SAP130. Furthermore it is understood that SAP130 includes a protein that binds specifically to an antibody that binds specifically to the protein of SEQ ID NO. 15. A gene encoding the SAP130 protein of SEQ ID NO. of SEQ ID NO. 15 is set forth in SEQ ID NO. 14.

SAP114 includes a protein having the amino acid sequence set forth in SEQ ID NO. 17 and variants thereof. Variants include (a) a protein having at least 85% sequence identity, more preferably at least 90% sequence identity to the amino acid sequence set forth in SEQ ID NO. 17, or (b) a protein fragment comprising at least 25 consecutive amino acids set forth in SEQ ID NO. 17. The fragments optionally have at least 75%, optionally at least 85%, and optionally at least 90% of the biological activity of the full length protein set forth in SEQ ID NO. 17. It is understood that the variants include allelic variants of SAP 114. Furthermore it is understood that SAP114 includes a protein that binds specifically to an antibody that binds specifically to the protein of SEQ ID NO. 17. A gene encoding the SAP 114 protein of SEQ ID NO. 17 is set forth in SEQ ID NO. 16.

SAP62 includes a protein having the amino acid sequence set forth in SEQ ID NO. 19 and variants thereof. Variants include (a) a protein having at least 85% sequence identity, more preferably at least 90% sequence identity to the amino acid sequence set forth in SEQ ID NO. 19, or (b) a protein fragment comprising at least 25 consecutive amino acids set forth in SEQ ID NO. 19. The fragments optionally have at least 75%, optionally at least 85%, and optionally at least 90% of the biological activity of the full length protein set forth in SEQ ID NO. 19. It is understood that the variants include allelic variants of SAP62. Furthermore it is understood that SAP62 includes a protein that binds specifically to an antibody that binds specifically to the protein of SEQ ID NO. 19. A gene encoding the SAP62 protein of SEQ ID NO. 19 is set forth in SEQ ID NO. 18.

SAP61 includes a protein having the amino acid sequence set forth in SEQ ID NO. 21 and variants thereof. Variants include (a) a protein having at least 85% sequence identity, more preferably at least 90% sequence identity to the amino acid sequence set forth in SEQ ID NO. 21, or (b) a protein fragment comprising at least 25 consecutive amino acids set forth in SEQ ID NO. 21. The fragments optionally have at least 75%, optionally at least 85%, and optionally at least 90% of the biological activity of the full length protein set forth in SEQ ID NO. 21. It is understood that the variants include allelic variants of SAP61. Furthermore it is understood that SAP61 includes a protein that binds specifically to an antibody that binds specifically to the protein of SEQ ID NO. 21. A gene encoding the SAP61 protein of SEQ ID NO. 21 is set forth in SEQ ID NO. 20.

SAP49 includes a protein having the amino acid sequence set forth in SEQ ID NO. 23 and variants thereof. Variants include (a) a protein having at least 85% sequence identity, more preferably at least 90% sequence identity to the amino acid sequence set forth in SEQ ID NO. 23, or (b) a protein fragment comprising at least 25 consecutive amino acids set forth in SEQ ID NO. 23. The fragments optionally have at least 75%, optionally at least 85%, and optionally at least 90% of the biological activity of the full length protein set forth in SEQ ID NO. 23. It is understood that the variants include allelic variants of SAP49. Furthermore it is understood that SAP49 includes a protein that binds specifically to an antibody that binds specifically to the protein of SEQ ID NO. 23. A gene encoding the SAP49 protein of SEQ ID NO. 23 is set forth in SEQ ID NO. 22.

U2 snRNP A′ includes a protein having the amino acid sequence set forth in SEQ ID NO. 25 and variants thereof. Variants include (a) a protein having at least 85% sequence identity, more preferably at least 90% sequence identity to the amino acid sequence set forth in SEQ ID NO. 25, or (b) a protein fragment comprising at least 25 consecutive amino acids set forth in SEQ ID NO. 25. The fragments optionally have at least 75%, optionally at least 85%, and optionally at least 90% of the biological activity of the full length protein set forth in SEQ ID NO. 25. It is understood that the variants include allelic variants of U2 snRNP A′. Furthermore it is understood that U2 snRNP A′ includes a protein that binds specifically to an antibody that binds specifically to the protein of SEQ ID NO. 25. A gene encoding the U2 snRNP A′ protein of SEQ ID NO. 25 is set forth in SEQ ID NO. 24.

p14 includes a protein having the amino acid sequence set forth in SEQ ID NO. 27 and variants thereof. Variants include (a) a protein having at least 85% sequence identity, more preferably at least 90% sequence identity to the amino acid sequence set forth in SEQ ID NO. 27, or (b) a protein fragment comprising at least 25 consecutive amino acids set forth in SEQ ID NO. 27. The fragments optionally have at least 75%, optionally at least 85%, and optionally at least 90% of the biological activity of the full length protein set forth in SEQ ID NO. 27. It is understood that the variants include allelic variants of p 14. Furthermore it is understood that p14 includes a protein that binds specifically to an antibody that binds specifically to the protein of SEQ ID NO. 27. A gene encoding the p14 protein of SEQ ID NO. 27 is set forth in SEQ ID NO. 26.

U2AF35 includes a protein having the amino acid sequence set forth in SEQ ID NO. 29 and variants thereof. Variants include (a) a protein having at least 85% sequence identity, more preferably at least 90% sequence identity to the amino acid sequence set forth in SEQ ID NO. 29, or (b) a protein fragment comprising at least 25 consecutive amino acids set forth in SEQ ID NO 29. The fragments optionally have at least 75%, optionally at least 85%, and optionally at least 90% of the biological activity of the full length protein set forth in SEQ ID NO. 29. It is understood that the variants include allelic variants of U2AF35. Furthermore it is understood that U2AF35 includes a protein that binds specifically to an antibody that binds specifically to the protein of SEQ ID NO. 29. A gene encoding the U2AF35 protein of SEQ ID NO. 29 is set forth in SEQ ID NO. 28.

U2AF65 includes a protein having the amino acid sequence set forth in SEQ ID NO. 31 and variants thereof. Variants include (a) a protein having at least 85% sequence identity, more preferably at least 90% sequence identity to the amino acid sequence set forth in SEQ ID NO. 31, or (b) a protein fragment comprising at least 25 consecutive amino acids set forth in SEQ ID NO. 31. The fragments optionally have at least 75%, optionally at least 85%, and optionally at least 90% of the biological activity of the full length protein set forth in SEQ ID NO. 31. It is understood that the variants include allelic variants of U2AF65. Furthermore it is understood that U2AF65 includes a protein that binds specifically to an antibody that binds specifically to the protein of SEQ ID NO. 31. A gene encoding the U2AF65 protein of SEQ ID NO. 31 is set forth in SEQ ID NO. 30.

U2AF 1-RS2 includes a protein having the amino acid sequence set forth in SEQ ID NO. 33 and variants thereof. Variants include (a) a protein having at least 85% sequence identity, more preferably at least 90% sequence identity to the amino acid sequence set forth in SEQ ID NO. 33, or (b) a protein fragment comprising at least 25 consecutive amino acids set forth in SEQ ID NO 33. The fragments optionally have at least 75%, optionally at least 85%, and optionally at least 90% of the biological activity of the full length protein set forth in SEQ ID NO. 33. It is understood that the variants include allelic variants of U2AF1-RS2. Furthermore it is understood that U2AF1-RS2 includes a protein that binds specifically to an antibody that binds specifically to the protein of SEQ ID NO. 33. A gene encoding the U2AF1-RS2 protein of SEQ ID NO. 33 is set forth in SEQ ID NO. 32.
hPrp5p includes a protein having the amino acid sequence set forth in SEQ ID NO. 35 and variants thereof. Variants include (a) a protein having at least 85% sequence identity, more preferably at least 90% sequence identity to the amino acid sequence set forth in SEQ ID NO. 35, or (b) a protein fragment comprising at least 25 consecutive amino acids set forth in SEQ ID NO. 35. The fragments optionally have at least 75%, optionally at least 85%, and optionally at least 90% of the biological activity of the full length protein set forth in SEQ ID NO. 35. It is understood that the variants include allelic variants of hPrpSp. Furthermore it is understood that hPrp5p includes a protein that binds specifically to an antibody that binds specifically to the protein of SEQ ID NO. 35. A gene encoding the hPrp5p protein of SEQ ID NO. 35 is set forth in SEQ ID NO. 34.
hprp19 includes a protein having the amino acid sequence set forth in SEQ ID NO. 37 and variants thereof. Variants include (a) a protein having at least 85% sequence identity, more preferably at least 90% sequence identity to the amino acid sequence set forth in SEQ ID NO. 37, or (b) a protein fragment comprising at least 25 consecutive amino acids set forth in SEQ ID NO. 37. The fragments optionally have at least 75%, optionally at least 85%, and optionally at least 90% of the biological activity of the full length protein set forth in SEQ ID NO. 37. It is understood that the variants include allelic variants of hPrp19. Furthermore it is understood that hPrp19 includes a protein that binds specifically to an antibody that binds specifically to the protein of SEQ ID NO. 37. A gene encoding the hPrp19 protein of SEQ ID NO. 37 is set forth in SEQ ID NO. 36.

HuR includes a protein having the amino acid sequence set forth in SEQ ID NO. 39 and variants thereof. Variants include (a) a protein having at least 85% sequence identity, more preferably at least 90% sequence identity to the amino acid sequence set forth in SEQ ID NO. 39, or (b) a protein fragment comprising at least 25 consecutive amino acids set forth in SEQ ID NO. 39. The fragments optionally have at least 75%, optionally at least 85%, and optionally at least 90% of the biological activity of the full length protein set forth in SEQ ID NO. 39. It is understood that the variants include allelic variants of HuR. Furthermore it is understood that HuR includes a protein that binds specifically to an antibody that binds specifically to the protein of SEQ ID NO. 39. A gene encoding the HuR protein of SEQ ID NO. 39 is set forth in SEQ ID NO. 38.

ALY includes a protein having the amino acid sequence set forth in SEQ ID NO. 41 and variants thereof. Variants include (a) a protein having at least 85% sequence identity, more preferably at least 90% sequence identity to the amino acid sequence set forth in SEQ ID NO. 41, or (b) a protein fragment comprising at least 25 consecutive amino acids set forth in SEQ ID NO. 41. The fragments optionally have at least 75%, optionally at least 85%, and optionally at least 90% of the biological activity of the full length protein set forth in SEQ ID NO. 41. It is understood that the variants include allelic variants of ALY. Furthermore it is understood that ALY includes a protein that binds specifically to an antibody that binds specifically to the protein of SEQ ID NO. 41. A gene encoding the ALY protein of SEQ ID NO. 41 is set forth in SEQ ID NO. 40.

SR140 includes a protein having the amino acid sequence set forth in SEQ ID NO. 43 and variants thereof. Variants include (a) a protein having at least 85% sequence identity, more preferably at least 90% sequence identity to the amino acid sequence set forth in SEQ ID NO. 43, or (b) a protein fragment comprising at least 25 consecutive amino acids set forth in SEQ ID NO. 43. The fragments optionally have at least 75%, optionally at least 85%, and optionally at least 90% of the biological activity of the full length protein set forth in SEQ ID NO. 43. It is understood that the variants include allelic variants of SR140. Furthermore it is understood that SR140 includes a protein that binds specifically to an antibody that binds specifically to the protein of SEQ ID NO. 43. A gene encoding the SR140 protein of SEQ ID NO. 43 is set forth in SEQ ID NO. 42.

CHERP includes a protein having the amino acid sequence set forth in SEQ ID NO. 45 and variants thereof. Variants include (a) a protein having at least 85% sequence identity, more preferably at least 90% sequence identity to the amino acid sequence set forth in SEQ ID NO. 45, or (b) a protein fragment comprising at least 25 consecutive amino acids set forth in SEQ ID NO. 45. The fragments optionally have at least 75%, optionally at least 85%, and optionally at least 90% of the biological activity of the full length protein set forth in SEQ ID NO. 45. It is understood that the variants include allelic variants of CHERP. Furthermore it is understood that CHERP includes a protein that binds specifically to an antibody that binds specifically to the protein of SEQ ID NO. 45. A gene encoding the CHERP protein of SEQ ID NO. 45 is set forth in SEQ ID NO. 44.

hPrp43 includes a protein having the amino acid sequence set forth in SEQ ID NO. 47 and variants thereof. Variants include (a) a protein having at least 85% sequence identity, more preferably at least 90% sequence identity to the amino acid sequence set forth in SEQ ID NO. 47, or (b) a protein fragment comprising at least 25 consecutive amino acids set forth in SEQ ID NO. 47. The fragments optionally have at least 75%, optionally at least 85%, and optionally at least 90% of the biological activity of the full length protein set forth in SEQ ID NO. 47. It is understood that the variants include allelic variants of hPrp43. Furthermore it is understood that hPrp43 includes a protein that binds specifically to an antibody that binds specifically to the protein of SEQ ID NO. 47. A gene encoding the hPrp43 protein is of SEQ ID NO. 47 set forth in SEQ ID NO. 46.

HSP75 includes a protein having the amino acid sequence set forth in SEQ ID NO. 49 and variants thereof. Variants include (a) a protein having at least 85% sequence identity, more preferably at least 90% sequence identity to the amino acid sequence set forth in SEQ ID NO. 49, or (b) a protein fragment comprising at least 25 consecutive amino acids set forth in SEQ ID NO. 49. The fragments optionally have at least 75%, optionally at least 85%, and optionally at least 90% of the biological activity of the full length protein set forth in SEQ ID NO. 49. It is understood that the variants include allelic variants of HSP75. Furthermore it is understood that HSP75 includes a protein that binds specifically to an antibody that binds specifically to the protein of SEQ ID NO. 49. A gene encoding the HSP75 protein of SEQ ID NO. 49 is set forth in SEQ ID NO. 48.

PUF60 includes a protein having the amino acid sequence set forth in SEQ ID NO. 51 and variants thereof. Variants include (a) a protein having at least 85% sequence identity, more preferably at least 90% sequence identity to the amino acid sequence set forth in SEQ ID NO. 51, or (b) a protein fragment comprising at least 25 consecutive amino acids set forth in SEQ ID NO. 51. The fragments optionally have at least 75%, optionally at least 85%, and optionally at least 90% of the biological activity of the full length protein set forth in SEQ ID NO. 51. It is understood that the variants include allelic variants of PUF60. Furthermore it is understood that PUF60 includes a protein that binds specifically to an antibody that binds specifically to the protein of SEQ ID NO. 51. A gene encoding the PUF60 protein is of SEQ ID NO. 51 set forth in SEQ ID NO. 50.

Hsp60 includes a protein having the amino acid sequence set forth in SEQ ID NO. 53 and variants thereof. Variants include (a) a protein having at least 85% sequence identity, more preferably at least 90% sequence identity to the amino acid sequence set forth in SEQ ID NO. 53, or (b) a protein fragment comprising at least 25 consecutive amino acids set forth in SEQ ID NO. 53. The fragments optionally have at least 75%, optionally at least 85%, and optionally at least 90% of the biological activity of the full length protein set forth in SEQ ID NO. 53. It is understood that the variants include allelic variants of Hsp60. Furthermore it is understood that Hsp60 includes a protein that binds specifically to an antibody that binds specifically to the protein of SEQ ID NO. 53. A gene encoding the Hsp60 protein of SEQ ID NO. 53 is set forth in SEQ ID NO. 52.

SPF45 includes a protein having the amino acid sequence set forth in SEQ ID NO. 55 and variants thereof. Variants include (a) a protein having at least 85% sequence identity, more preferably at least 90% sequence identity to the amino acid sequence set forth in SEQ ID NO. 55, or (b) a protein fragment comprising at least 25 consecutive amino acids set forth in SEQ ID NO. 55. The fragments optionally have at least 75%, optionally at least 85%, and optionally at least 90% of the biological activity of the full length protein set forth in SEQ ID NO. 55. It is understood that the variants include allelic variants of SPF45. Furthermore it is understood that SPF45 includes a protein that binds specifically to an antibody that binds specifically to the protein of SEQ ID NO. 55. A gene encoding the SPF45 protein of SEQ ID NO. 55 is set forth in SEQ ID NO. 54.

BRAF35 includes a protein having the amino acid sequence set forth in SEQ ID NO. 57 and variants thereof. Variants include (a) a protein having at least 85% sequence identity, more preferably at least 90% sequence identity to the amino acid sequence set forth in SEQ ID NO. 57, or (b) a protein fragment comprising at least 25 consecutive amino acids set forth in SEQ ID NO. 57. The fragments optionally have at least 75%, optionally at least 85%, and optionally at least 90% of the biological activity of the full length protein set forth in SEQ ID NO. 57. It is understood that the variants include allelic variants of BRAF35. Furthermore it is understood that BRAF35 includes a protein that binds specifically to an antibody that binds specifically to the protein of SEQ ID NO. 57. A gene encoding the BRAF35 protein of SEQ ID NO. 57 is set forth in SEQ ID NO. 56.

SF2/ASF includes a protein having the amino acid sequence set forth in SEQ ID NO. 59 and variants thereof. Variants include (a) a protein having at least 85% sequence identity, more preferably at least 90% sequence identity to the amino acid sequence set forth in SEQ ID NO. 59, or (b) a protein fragment comprising at least 25 consecutive amino acids set forth in SEQ ID NO. 59. The fragments optionally have at least 75%, optionally at least 85%, and optionally at least 90% of the biological activity of the full length protein set forth in SEQ ID NO. 59. It is understood that the variants include allelic variants of SF2/ASF. Furthermore it is understood that SF2/ASF includes a protein that binds specifically to an antibody that binds specifically to the protein of SEQ ID NO. 59. A gene encoding the SF2/ASF protein of SEQ ID NO. 59 is set forth in SEQ ID NO. 58.

SF3b14b includes a protein having the amino acid sequence set forth in SEQ ID NO. 61 and variants thereof. Variants include (a) a protein having at least 85% sequence identity, more preferably at least 90% sequence identity to the amino acid sequence set forth in SEQ ID NO. 61, or (b) a protein fragment comprising at least 25 consecutive amino acids set forth in SEQ ID NO. 61. The fragments optionally have at least 75%, optionally at least 85%, and optionally at least 90% of the biological activity of the full length protein set forth in SEQ ID NO. 61. It is understood that the variants include allelic variants of SF3b14b. Furthermore it is understood that SF3b14b includes a protein that binds specifically to an antibody that binds specifically to the protein of SEQ ID NO. 61. A gene encoding the SF3b14b protein of SEQ ID NO. 61 is set forth in SEQ ID NO. 60.

SF3b10 includes a protein having the amino acid sequence set forth in SEQ ID NO. 63 and variants thereof. Variants include (a) a protein having at least 85% sequence identity, more preferably at least 90% sequence identity to the amino acid sequence set forth in SEQ ID NO. 63, or (b) a protein fragment comprising at least 25 consecutive amino acids set forth in SEQ ID NO. 63. The fragments optionally have at least 75%, optionally at least 85%, and optionally at least 90% of the biological activity of the full length protein set forth in SEQ ID NO. 63. It is understood that the variants include allelic variants of SF3b10. Furthermore it is understood that SF3b10 includes a protein that binds specifically to an antibody that binds specifically to the protein of SEQ ID NO. 63. A gene encoding the SF3b10 protein of SEQ ID NO. 63 is set forth in SEQ ID NO. 62.

SF3a120 includes a protein having the amino acid sequence set forth in SEQ ID NO. 65 and variants thereof. Variants include (a) a protein having at least 85% sequence identity, more preferably at least 90% sequence identity to the amino acid sequence set forth in SEQ ID NO. 65, or (b) a protein fragment comprising at least 25 consecutive amino acids set forth in SEQ ID NO. 65. The fragments optionally have at least 75%, optionally at least 85%, and optionally at least 90% of the biological activity of the full length protein set forth in SEQ ID NO. 65. It is understood that the variants include allelic variants of SF3a120. Furthermore it is understood that SF3a120 includes a protein that binds specifically to an antibody that binds specifically to the protein of SEQ ID NO. 65. A gene encoding the SF3a120 protein of SEQ ID NO. 65 is set forth in SEQ ID NO. 64.

SF3a66 includes a protein having the amino acid sequence set forth in SEQ ID NO. 67 and variants thereof. Variants include (a) a protein having at least 85% sequence identity, more preferably at least 90% sequence identity to the amino acid sequence set forth in SEQ ID NO. 67, or (b) a protein fragment comprising at least 25 consecutive amino acids set forth in SEQ ID NO. 65. The fragments optionally have at least 75%, optionally at least 85%, and optionally at least 90% of the biological activity of the full length protein set forth in SEQ ID NO. 67. It is understood that the variants include allelic variants of SF3a66. Furthermore it is understood that SF3a66 includes a protein that binds specifically to an antibody that binds specifically to the protein of SEQ ID NO. 67. A gene encoding the SF3a66 protein of SEQ ID NO. 67 is set forth in SEQ ID NO. 66.

SF3a60 includes a protein having the amino acid sequence set forth in SEQ ID NO. 69 and variants thereof. Variants include (a) a protein having at least 85% sequence identity, more preferably at least 90% sequence identity to the amino acid sequence set forth in SEQ ID NO. 69, or (b) a protein fragment comprising at least 25 consecutive amino acids set forth in SEQ ID NO. 69. The fragments optionally have at least 75%, optionally at least 85%, and optionally at least 90% of the biological activity of the full length protein set forth in SEQ ID NO. 69. It is understood that the variants include allelic variants of SF3a60. Furthermore it is understood that SF3a60 includes a protein that binds specifically to an antibody that binds specifically to the protein of SEQ ID NO. 69. A gene encoding the SF3a60 protein of SEQ ID NO. 69 is set forth in SEQ ID NO. 68.

SPF30 includes a protein having the amino acid sequence set forth in SEQ ID NO. 71 and variants thereof. Variants include (a) a protein having at least 85% sequence identity, more preferably at least 90% sequence identity to the amino acid sequence set forth in SEQ ID NO. 71, or (b) a protein fragment comprising at least 25 consecutive amino acids set forth in SEQ ID NO. 71. The fragments optionally have at least 75%, optionally at least 85%, and optionally at least 90% of the biological activity of the full length protein set forth in SEQ ID NO. 71. It is understood that the variants include allelic variants of SPF30. Furthermore it is understood that SPF30 includes a protein that binds specifically to an antibody that binds specifically to the protein of SEQ ID NO. 71. A gene encoding the SPF30 protein of SEQ ID NO. 71 is set forth in SEQ ID NO. 70.

SmB/B′ includes a protein having the amino acid sequence set forth in SEQ ID NO. 73 and variants thereof. Variants include (a) a protein having at least 85% sequence identity, more preferably at least 90% sequence identity to the amino acid sequence set forth in SEQ ID NO. 73, or (b) a protein fragment comprising at least 25 consecutive amino acids set forth in SEQ ID NO. 73. The fragments optionally have at least 75%, optionally at least 85%, and optionally at least 90% of the biological activity of the full length protein set forth in SEQ ID NO. 73. It is understood that the variants include allelic variants of SmB/B′. Furthermore it is understood that SmB/B′ includes a protein that binds specifically to an antibody that binds specifically to the protein of SEQ ID NO. 73. A gene encoding the SmB/B′ protein of SEQ ID NO. 73 is set forth in SEQ ID NO. 72.

SmD3 includes a protein having the amino acid sequence set forth in SEQ ID NO. 75 and variants thereof. Variants include (a) a protein having at least 85% sequence identity, more preferably at least 90% sequence identity to the amino acid sequence set forth in SEQ ID NO. 75, or (b) a protein fragment comprising at least 25 consecutive amino acids set forth in SEQ ID NO. 75. The fragments optionally have at least 75%, optionally at least 85%, and optionally at least 90% of the biological activity of the full length protein set forth in SEQ ID NO. 75. It is understood that the variants include allelic variants of SmD3. Furthermore it is understood that SmD3 includes a protein that binds specifically to an antibody that binds specifically to the protein of SEQ ID NO. 75. A gene encoding the SmD3 protein of SEQ ID NO. 75 is set forth in SEQ ID NO. 74.

SmD2 includes a protein having the amino acid sequence set forth in SEQ ID NO. 77 and variants thereof. Variants include (a) a protein having at least 85% sequence identity, more preferably at least 90% sequence identity to the amino acid sequence set forth in SEQ ID NO. 77, or (b) a protein fragment comprising at least 25 consecutive amino acids set forth in SEQ ID NO. 77. The fragments optionally have at least 75%, optionally at least 85%, and optionally at least 90% of the biological activity of the full length protein set forth in SEQ ID NO. 77. It is understood that the variants include allelic variants of SmD2. Furthermore it is understood that SmD2 includes a protein that binds specifically to an antibody that binds specifically to the protein of SEQ ID NO. 77. A gene encoding the SmD2 protein of SEQ ID NO. 77 is set forth in SEQ ID NO. 76.

SmD1 includes a protein having the amino acid sequence set forth in SEQ ID NO. 79 and variants thereof. Variants include (a) a protein having at least 85% sequence identity, more preferably at least 90% sequence identity to the amino acid sequence set forth in SEQ ID NO. 79, or (b) a protein fragment comprising at least 25 consecutive amino acids set forth in SEQ ID NO. 79. The fragments optionally have at least 75%, optionally at least 85%, and optionally at least 90% of the biological activity of the full length protein set forth in SEQ ID NO. 79. It is understood that the variants include allelic variants of SmD1. Furthermore it is understood that SmD1 includes a protein that binds specifically to an antibody that binds specifically to the protein of SEQ ID NO. 79. A gene encoding the SmD1 protein of SEQ ID NO. 79 is set forth in SEQ ID NO. 78.

SmE includes a protein having the amino acid sequence set forth in SEQ ID NO. 81 and variants thereof. Variants include (a) a protein having at least 85% sequence identity, more preferably at least 90% sequence identity to the amino acid sequence set forth in SEQ ID NO. 81, or (b) a protein fragment comprising at least 25 consecutive amino acids set forth in SEQ ID NO. 81. The fragments optionally have at least 75%, optionally at least 85%, and optionally at least 90% of the biological activity of the full length protein set forth in SEQ ID NO. 81. It is understood that the variants include allelic variants of SmE. Furthermore it is understood that SmE includes a protein that binds specifically to an antibody that binds specifically to the protein of SEQ ID NO. 81. A gene encoding the SmE protein of SEQ ID NO. 81 is set forth in SEQ ID NO. 80.

SmF includes a protein having the amino acid sequence set forth in SEQ ID NO. 83 and variants thereof. Variants include (a) a protein having at least 85% sequence identity, more preferably at least 90% sequence identity to the amino acid sequence set forth in SEQ ID NO. 83, or (b) a protein fragment comprising at least 25 consecutive amino acids set forth in SEQ ID NO. 83. The fragments optionally have at least 75%, optionally at least 85%, and optionally at least 90% of the biological activity of the full length protein set forth in SEQ ID NO. 83. It is understood that the variants include allelic variants of SmF. Furthermore it is understood that SmF includes a protein that binds specifically to an antibody that binds specifically to the protein of SEQ ID NO. 83. A gene encoding the SmF protein of SEQ ID NO. 83 is set forth in SEQ ID NO. 82.

SmG includes a protein having the amino acid sequence set forth in SEQ ID NO. 85 and variants thereof. Variants include (a) a protein having at least 85% sequence identity, more preferably at least 90% sequence identity to the amino acid sequence set forth in SEQ ID NO. 85, or (b) a protein fragment comprising at least 25 consecutive amino acids set forth in SEQ ID NO. 85. The fragments optionally have at least 75%, optionally at least 85%, and optionally at least 90% of the biological activity of the full length protein set forth in SEQ ID NO. 85. It is understood that the variants include allelic variants of SmG. Furthermore it is understood that SmG includes a protein that binds specifically to an antibody that binds specifically to the protein of SEQ ID NO. 85. A gene encoding the SmG protein of SEQ ID NO. 85 is set forth in SEQ ID NO. 84.

In order to produce variants of the disclosed proteins or other proteins present in the U2 spliceosomal particles that may also serve as identifiers for the U2 particle, any one or more of the naturally-occurring protein sequences may be used as a reference sequence to determine whether a candidate sequence possesses sufficient amino acid similarity to have a reasonable expectation of success in the methods of the present invention.

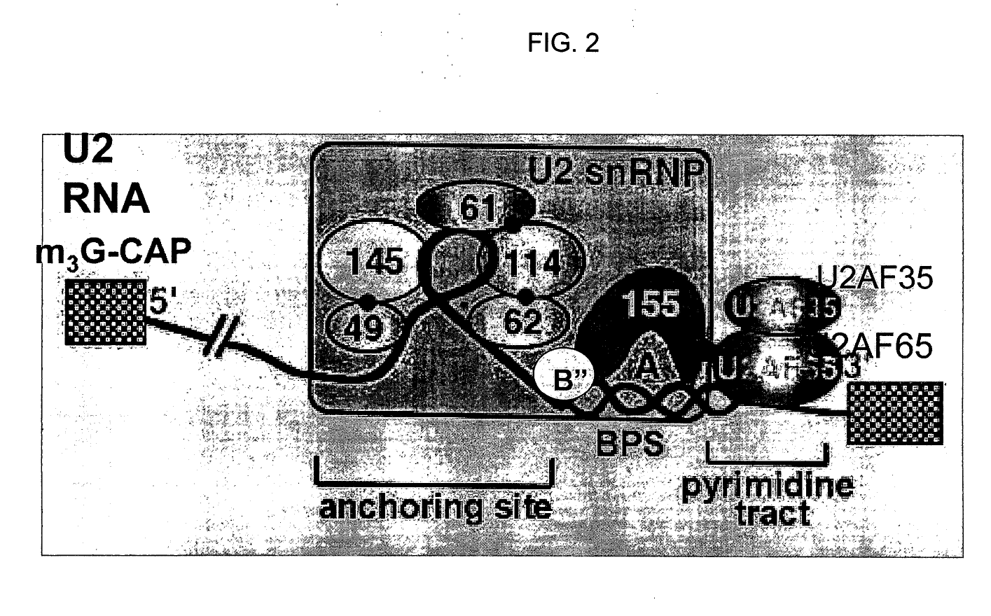

To determine whether a candidate peptide region has the requisite percentage identity to a reference polypeptide or peptide oligomer, the candidate amino acid sequence and the reference amino acid sequence are first aligned using the dynamic programming algorithm described in Smith and Waterman (1981), J. Mol. Biol. 147:195-197, in combination with the BLOSUM62 substitution matrix described in FIG. 2 of Henikoff and Henikoff (1992), “Amino acid substitution matrices from protein blocks”, Proc. Natl. Acad. Sci. USA (1992), 89:10915-10919. For the present invention, an appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent identity score may be calculated. To calculate a percent identity, the aligned amino acids of each sequence are compared sequentially. If the amino acids are non-identical, the pairwise identity score is zero; otherwise the pairwise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

FIG. 2 shows a schematic illustration of the U2 particle structure. The U2 snRNA gene is a reiterated sequence occurring on several chromosomes and having known pseudogenes. This reiteration feature gives assays an inherent sensitivity. Several of the known RNA binding components are shown. FIG. 3 shows a drawing of a mature U2 snRNA, aligned as it is predicted to appear at physiologic temperature and tonicity. The stem-loop IV, which binds the B″ protein, appears on the right hand side of the drawing. Also illustrated in FIGS. 2 and 3 is the 2,2,7-trimethylguanosine “CAP” structure of the U2 snRNA. The CAP structure is unique to U RNAs, and consists of a 5′ to 5′ phosphotriester link between the leading guanidine residue and the adenosine that follows it. The leading guanidine is methylated twice at position 2 and once at position 7. This CAP structure is antigenic. Anti-CAP antibodies are available commercially from a variety of sources. The CAP ligand and commercially available anti-CAP antibodies permit capture of U2 snRNA-containing complexes from human serum.

Sequences for exemplary primers useful in reverse transcription and PCR amplification of the human U2 snRNA sequence are set forth in SEQ ID NO. 86 and SEQ ID NO. 87. In combination with commercially-available RT-PCR kits, the RT-PCR primers can be used to amplify U2 snRNA from a human body fluid, such as serum. The RT-PCR primers are unsubstituted, but could be adapted for use in a quantitative real-time RT-PCR system. This adaptation would allow a direct and quantitative comparison of the test and control populations in the study of breast cancer and other diseases.

The U2 snRNA or other U2 particle component to be detected preferably is purified prior to the detection step. For example, one component of the U2 particle can be used as a target for the purification. Thereafter, a second different component of the U2 particle can be analyzed to determine whether an individual has or is at risk of developing cancer, for example, breast cancer. It is understood that the cancer includes both cancerous and pre-cancerous lesions.

Purification can involve a binding moiety that recognizes the 2,2,7 trimethylguanosine CAP, such as a natural or recombinant snurportin (SPN-1) protein or a polyclonal or monoclonal antibody. Alternatively, purification can involve a binding moiety that recognizes the sequence of the snRNA or a protein directly or indirectly associated with the snRNA, such as U2 snRNP B″ or another component of the U2 particle. Streptavidin, avidin, or a similar compound can be used to capture a biotinylated form of the U2 complex that may circulate. Additionally, morpholino antisense oligos can be used to capture components of the U2 particle. Antibodies raised against the general U particle family, including anti-Sm, anti-SMN, anti-ImportinB, anti-snurportin, anti-Ran, or anti Ran-GTP antibodies can also be used.

Elution of U2 particle components from a binding moiety used for purification is not generally required prior to detection. For example, an RT-PCR reaction to amplify U2 snRNA or a fragment thereof can be performed without separating a U2 snRNA from an antibody to the 2,2,7-trimethylguanosine moiety prior to commencing the reaction. Elution, however, often is preferred. Elution from a binding moiety recognizing 2,2,7-trimethylguanosine can be achieved, for example, by administering a competing ligand, such as free 7-methylguanosine. Elution from antibodies to other components can be achieved by disrupting the antigen-antibody interaction, for example, by reducing the pH.

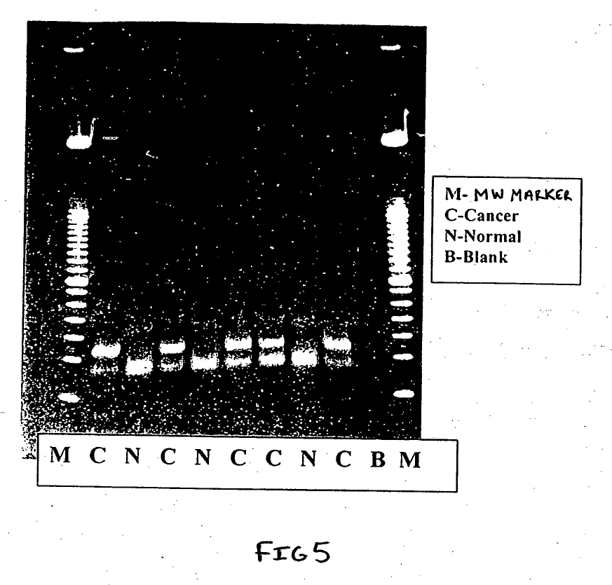

One embodiment of the purification detection process involves capturing a U2 particle via one or more antibodies immobilized on a solid support, for example, beads packed within a column. After binding, components of the U2 particle are eluted and submitted for analysis. Briefly, the samples can be analyzed by amplification of U2 SnRNA by RT-PCR. Following amplification, the amplification products can be fractionated by polyacrylamide gel electrophoresis and the bands visualized by ethedium bromide staining (see FIG. 5). As shown in FIG. 5, higher levels of amplicon were observed in samples from women with breast cancer relative to samples from healthy women. In that experiment, the sensitivity and specificity were 100%.

Exemplary protocols for detecting target proteins and nucleic acids present in the U2 particle are described in the following sections.

I. Exemplary Protocols for Detecting a Target Nucleic Acid

A target nucleic acid molecule, for example, U2 snRNA may be detected using a labeled binding moiety capable of specifically binding the target nucleic acid. The binding moiety may comprise, for example, a protein, a nucleic acid or a peptide nucleic acid. Additionally, a target nucleic acid may be detected by conducting, for example, a Northern blot analysis using labeled oligonucleotides, for example, nucleic acid fragments complementary to and capable of hybridizing specifically with at least a portion of a target nucleic acid. The probes hybridize with complementary nucleic acid sequences presented in the test specimen, and can provide exquisite specificity. A short, well-defined probe for a single unique sequence is most precise and preferred. Larger probes are generally less specific. While an oligonucleotide of any length may hybridize to a target such as U2 snRNA, oligonucleotides typically within the range of 8-100 nucleotides, preferably within the range of 15-50 nucleotides, are envisioned to be most useful in standard hybridization assays. Choices of probe length and sequence allow one to choose the degree of specificity desired. Hybridization preferably is carried out at a temperature from 50° to 65° C. in a high salt buffer solution, formamide or other agents to set the degree of complementarity required. Furthermore, the state of the art is such that probes can be manufactured to recognize essentially any DNA or RNA sequence. For additional particulars, see, for example, Guide to Molecular Techniques, Berger et al., Methods of Enzymology, Vol. 152, 1987.

Because the complete nucleotide sequence encoding the U2 snRNA is known and/or can be determined readily using techniques well known in the art, complementary oligonucleotides or peptide nucleic acids which hybridize specifically with any portion of the U2 snRNA transcript or non-coding sequences can be prepared using conventional oligonucleotide and peptide nucleic acid synthesis methodologies. A variety of sequence lengths of oligonucleotide or peptide nucleic acid may be used to hybridize to U2 snRNA transcripts. However, very short sequences (e.g., sequences containing less than 8-15 nucleobases) may bind the target nucleic acid with less specificity.

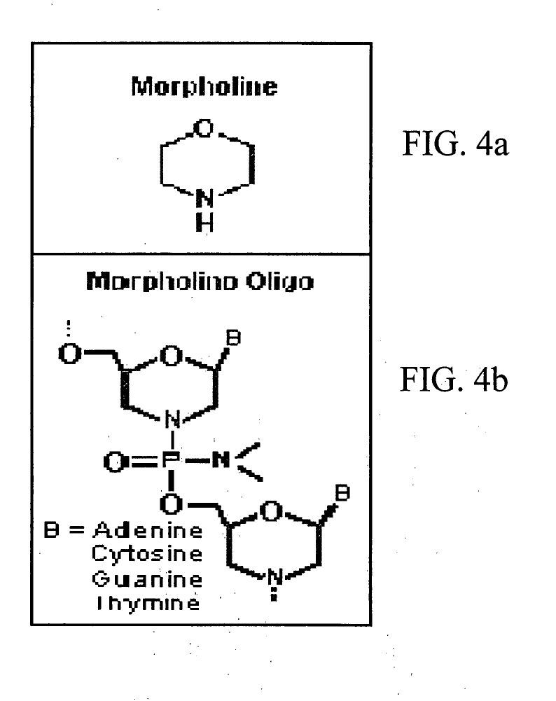

Certain oligonucleotides suffer from such limitations as poor specificity, instability, unpredictable targeting and undesirable non-antisense effects. Nucleic acid analogs containing, for example, morpholino groups, can overcome some of these limitations. Morpholino containing oligonucleotides are assembled from four different morpholino subunits, each of which contains one of the four genetic bases (adenosine, thymidine, guanosine or cytosine) linked to a 6-membered morpholine ring (see FIG. 4A). Eighteen to 25 subunits of the four subunit types are joined in a specific order by non-ionic phosphorodiamidate intersubunit linkages to give a morpholino oligonucleotide. FIG. 4B shows a short segment of a morpholino oligonucleotide, comprising two subunits joined by an intersubunit linkage. The morpholino oligonucleotides with their 6-membered morpholine backbone moieties joined by non-ionic linkages afford beneficial properties relative to RNA, DNA, and their analogs having 5-membered ribose or deoxyribose backbone moieties joined by ionic linkages. Morpholinos have desireable qualities in terms of serum stability and hybridization stringency.

A wide variety of different labels coupled to probes or antibodies may be employed in the assays described in this section and in the following section. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescers. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′hydroxyl termini within either strand of the double-stranded fragment. Simultaneously, an exonuclease removes the nucleotide residue from the 5′phosphoryl side of the nick. The sequence of replacement nucleotides is determined by the sequence of the opposite strand of the duplex. Thus, if labeled nucleotides are supplied, DNA polymerase will fill in the nick with the labeled nucleotides. Alternatively, nucleotide probes can be labeled by 3′end labeling. Furthermore, there are currently commercially available methods of labeling DNA with fluorescent molecules, catalysts, enzymes, or chemiluminescent materials. Biotin labeling kits are commercially available (Enzo Biochem, Inc.) under the Bio-Probe trade name. This type of system permits the probe to be coupled to avidin which in turn is labeled with, for example, a fluorescent molecule, enzyme, antibody, etc. For further disclosure regarding probe construction and technology, see, for example, Sambrook et al., Molecular Cloning, A Laboratory Manual (Cold Spring Harbor, N.Y., 1982).

The oligonucleotide selected for hybridizing to the target nucleic acid, whether synthesized chemically or by recombinant DNA methodologies, is isolated and purified using standard techniques and then preferably labeled (e.g., with ³⁵S or ³²P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridizing conditions, e.g., 50% formamide, 5×SSPE, 2× Denhardt's solution, 0.1% SDS at 42° C., as described in Sambrook et al. (1989) supra. The filter may then be washed using 2×SSPE, 0.1% SDS at 68° C., and more preferably using 0.1×SSPE, 0.1% SDS at 68° C. Other useful procedures known in the art include solution hybridization, and dot and slot RNA hybridization. Optionally, the amount of the target nucleic acid present in a sample then is quantitated by measuring the radioactivity of hybridized fragments, using standard procedures known in the art.

In addition, using a combination of appropriate oligonucleotide primers, the skilled artisan can determine the level of the target nucleic acid by standard polymerase chain reaction (PCR) procedures as described elsewhere herein, for example, by quantitative PCR. Conventional PCR based assays are discussed, for example, in Innes et al (1990) “PCR Protocols; A guide to methods and Applications”, Academic Press and Innes et al. (1995) “PCR Strategies” Academic Press, San Diego, Calif. For example, U2 snRNA is detectable using a fewer number of PCR cycles in the serum of women with breast cancer than in the serum of healthy women. Additionally, the nucleic acids encoding marker proteins may be detected using nucleic acid probes having a sequence complementary to at least a portion of the sequence encoding the marker protein.

II. Exemplary Protocols for Detecting a Target Protein

A cancer marker, such as a component of a U2 particle, may be detected, for example, by combining the marker with a binding moiety capable of specifically binding the marker. The binding moiety may comprise, for example, a member of a ligand-receptor pair, i.e., a pair of molecules capable of having a specific binding interaction. The binding moiety may comprise, for example, a member of a specific binding pair, such as antibody-antigen, enzyme-substrate, protein-nucleic acid, protein-protein, or other specific binding pair known in the art. Binding proteins may be designed which have enhanced affinity for a target. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector.

A cancer marker may also be detected using any of a wide range of immunoassay techniques available in the art. For example, the skilled artisan may employ a sandwich immunoassay format to detect a cancer marker in a body fluid sample. Alternatively, the skilled artisan may use conventional immuno-histochemical procedures for detecting the presence of the cancer marker in a tissue sample using one or more labeled binding proteins.

In a sandwich immunoassay, two antibodies capable of binding the marker generally are used, e.g., one immobilized onto a solid support, and one free in solution and labeled with a detectable chemical compound. Examples of chemical labels that may be used for the second antibody include radioisotopes, fluorescent compounds, and enzymes or other molecules that generate colored or electrochemically active products when exposed to a reactant or enzyme substrate. When a sample containing the marker is placed in this system, the marker binds to both the immobilized antibody and the labeled antibody, to form a “sandwich” immune complex on the support's surface. The complexed protein is detected by washing away non-bound sample components and excess labeled antibody, and measuring the amount of labeled antibody complexed to protein on the support's surface. Alternatively, the antibody free in solution, which can be labeled with a chemical moiety, for example, a hapten, may be detected by a third antibody labeled with a detectable moiety which binds the free antibody or, for example, the hapten coupled thereto.

Both the sandwich immunoassay and tissue immunohistochemical procedures are highly specific and very sensitive, provided that labels with good limits of detection are used. A detailed review of immunological assay design, theory and protocols can be found in numerous texts in the art, including “Practical Immunology”, Butt, W. R., ed., (1984) Marcel Dekker, New York and “Antibodies, A Laboratory Approach”, Harlow et al. eds., (1988) Cold Spring Harbor Laboratory.

In general, immunoassay design considerations include the preparation of antibodies (e.g., monoclonal or polyclonal antibodies) having sufficiently high binding specificity for the target to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target protein. The higher the antibody binding specificity, the lower the target protein concentration that can be detected. As used herein, the terms “specific binding” or “binding specifically” are understood to mean that the binding moiety, for example, a binding protein has a binding affinity for the target protein of greater than about 105 M⁻¹, more preferably greater than about 107 M⁻¹.

Antibodies to an isolated target may be generated using standard immunological procedures well known and described in the art. See, for example, “Practical Immunology” (1984) supra. Briefly, an isolated target is used to raise antibodies in a host, such as a mouse, goat or other suitable mammal. The marker protein is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells and available from, for example, Calbiochem Corp., San Diego, or Gibco, Grand Island, N.Y.). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target and have the desired binding affinity.

Antibody binding domains also may be produced biosynthetically and the amino acid sequence of the binding domain manipulated to enhance binding affinity with a preferred epitope on the target. Specific antibody methodologies are well understood and described in the literature. A more detailed description of their preparation can be found, for example, in “Practical Immunology” (1984) supra.

In addition, genetically engineered biosynthetic antibody binding sites, also known in the art as BABS or sFv's, may be used in the practice of the instant invention. Methods for making and using BABS comprising (i) non-covalently associated or disulfide bonded synthetic V_{H} and V_{L} dimers, (ii) covalently linked V_{H}-V_{L} single chain binding sites, (iii) individual V_{H} or V_{L} domains, or (iv) single chain antibody binding sites are disclosed, for example, in U.S. Pat. Nos. 5,091,513; 5,132,405; 4,704,692; and 4,946,778. Furthermore, BABS having requisite specificity for a cancer marker can be derived by phage antibody cloning from combinatorial gene libraries (see, for example, Clackson et al. (1991) Nature 352: 624-628). Briefly, phage each expressing on their coat surfaces BABS having immunoglobulin variable regions encoded by variable region gene sequences derived from mice pre-immunized with an isolated cancer marker, or a fragment thereof, are screened for binding activity against the immobilized marker. Phage which bind to the immobilized marker are harvested and the gene encoding the BABS is sequenced. The resulting nucleic acid sequences encoding the BABS of interest then may be expressed in conventional expression systems to produce the BABS protein.

Cancer markers may also be detected using gel electrophoresis techniques available in the art. In two-dimensional gel electrophoresis, proteins are separated first in a pH gradient gel according to their isoelectric point. The resulting gel then is placed on a second polyacrylamide gel, and the proteins separated according to molecular weight (see, for example, O'Farrell (1975) J. Biol. Chem. 250: 4007-4021).

One or more marker proteins may be detected by first isolating proteins from a sample obtained from an individual suspected of having cancer, and then separating the proteins by two-dimensional gel electrophoresis to produce a characteristic two-dimensional gel electrophoresis pattern. The pattern may then be compared with a standard gel pattern produced by separating, under the same or similar conditions, proteins isolated from normal or cancer cells. The standard gel pattern may be stored in, and retrieved from an electronic database of electrophoresis patterns. The presence of a cancer-associated protein in the two-dimensional gel provides an indication that the sample being tested was taken from a person with cancer. As with the other detection assays described herein, the detection of two or more proteins, for example, in the two-dimensional gel electrophoresis pattern further enhances the accuracy of the assay. The presence of a plurality, e.g., two to five, cancer-associated proteins on the two-dimensional gel provides an even stronger indication of the presence of a cancer in the individual. The assay thus permits the early detection and treatment of cancer.

Mass spectrometry may also be used to detect a marker protein. Preferred mass spectrometry methods include MALDI-TOF mass spectrometry and MALDI-TOF using derivatized chip surfaces (SELDI). Useful mass spectrometry methods for detecting a marker protein are described, for example, in U.S. Pat. Nos. 5,719,060; 5,894,063; 6,124,137; 6,207,370; 6,225,047; 6,281,493; 6,322,970; and 6,936,424. In these methods, the presence and/or amount of a particular marker protein in a separation profile can be monitored. Alternatively, the presence and/or amount of a plurality of marker proteins in a separation profile can be monitored. In such approaches, the separation profile of a marker protein or proteins derived from a test patient of unknown disposition may be compared against the separation profile of the marker protein or proteins derived from a control sample (for example, a negative control where an individual is confirmed not to have breast cancer or a positive control where individual(s) is or are have been having breast cancer). The amounts of one or more of the marker proteins in the test sample relative to the amount of the same or similar proteins in the control sample can be a diagnostic or prognostic indicator of whether the individual providing the test sample may have breast cancer and/or the severity of the breast cancer. For example, a result in which the amount of a particular marker protein in the separation protein from a test individual is less than or equal to the amount of marker protein in a negative control sample is indicative that the test individual does not have breast cancer. In contrast, a result in which the amount of a particular marker protein in the separation profile from a test individual is greater than the amount of the marker protein in a positive control sample is indicative that the test individual may have breast cancer.

The invention may be more completely understood by reference to the following non-limiting examples.

### EXAMPLES

### Example 1

### Detection of Free U2 SnRNP B″ in Serum

This Example describes the development of a sandwich immunoassay for detecting free U2 snRNP B″ protein in a sample that has been externalized from a nucleus, for example, by apoptosis or oxidative stress associated with cancer. Paired monoclonal antibodies were selected that recognize distinct epitopes on the U2 snRNP B″ protein.

ELISA microtiter plates were coated with a 1D5 capture antibody. The 1D5 capture antibody is a monoclonal antibody that was created using recombinant U2 snRNP B″ (see, SEQ ID NO. 3) as an antigen and binds to an epitope on U2 snRNP B″ that is different from the epitope bound by the 4G3 monoclonal antibody (available from, for example, Eurodiagnostika, The Netherlands). These plates then were blocked by incubation with bovine serum albumin (BSA) at a concentration of 2 μg/mL for 4 hours at room temperature. Then, 400 μL of serum sample was diluted in a mixture of normal human serum (NHS): phosphate-buffered saline (PBS) 1:1, at ratios of 1:1, 1:2, 1:4, and 1:8 of sample to diluent. The diluted sample was added to the plate and incubated for 1 hour at 37° C., after which the plate was washed 3 times with PBS. Subsequently, 400 μL of a biotinylated detection antibody, biotinylated 4G3 (obtained from Eurodiagnostika, The Netherlands) at a concentration of 0.2 μg/mL was added to the plate and incubated for 1 hour at 37° C. After incubation, the plate was washed 3 times with PBS. Following incubation, Streptavidin-horse radish peroxidase fusion protein (SA-HRP)(obtained from Jackson ImmunoResearch, Inc.) was added to plate and incubated for 15 minutes at room temperature.

100 μL of DAKO TMB Blue 1-Step Component Microwell Peroxidase (DAKO Corporation, Carpinteria California) was added to the plate to generate a signal, which was measured calorimetrically using a Spectramax Plus (Molecular Devices) spectrophotometer. The signals were scored for optical density at a wavelength of 450 nm and the concentration of U2 snRNP B″ was determined using a standard curve generated using known amounts of U2 snRNP B″. The standard curves were generated using 5 levels of recombinant U2 snRNP B″.

It is contemplated that concentrations of U2 snRNP B″ greater than a predetermined threshold value can be indicative of the presence of breast cancer in the donor.

### Example 2

### Detection of Complexed U2 snRNP B″ in Serum

This Example describes the development of a second sandwich immunoassay that recognizes U2 snRNP B″ when it was complexed to other proteins in a sample.

Paired monoclonal antibodies were selected that recognized distinct epitopes on the U2 snRNP B″ protein. ELISA microtiter plates were coated with a 1D5 capture antibody and blocked by incubation with bovine serum albumin (BSA) at a concentration of 2 μg/mL for 4 hours at room temperature.

The samples were first denatured with 2M urea to disrupt the U2 complex. Then, 400 μL of the denatured sample was diluted in mixture of normal human serum (NHS): phosphate-buffered saline (PBS) 1:1, at ratios of 1:1, 1:2, 1:4, and 1:8 of sample to diluent. The diluted sample was added to the plate and incubated with the plate for 1 hour at 37° C., after which the plate was washed 3 times with PBS. Subsequently, 400 μL of a biotinylated detection antibody, biotinylated 4G3 (Eurodiagnostika, The Netherlands) at a concentration of 0.2 μg/mL was added to the plate and incubated for 1 hour at 37° C. After incubation, the plate was washed 3 times with PBS. Following incubation, Streptavidin-horse radish peroxidase fusion protein (SA-HRP)(Jackson ImmunoResearch, Inc.) was added to plate and incubated for 15 minutes at room temperature.

100 μL of DAKO TMB Blue 1-Step Component Microwell Peroxidase (DAKO Corporation, Carpinteria California) was added to the plate to generate a signal, which was measured calorimetrically using a Spectramax Plus (Molecular Devices) spectrophotometer. The signals were scored for optical density at a wavelength of 450 nm. The resulting values were used to calculate the concentration of U2 snRNP B″ by interpolation from a calibration curve created using different concentrations of U2 snRNP B″. A level of U2 snRNP B″ higher than a determined threshold was indicative of cancer in the sample. The standard curves were generated using 5 levels of isolated U2 snRNP B″-associated complex. The minimum analytical detection limit was set at the signal level 3 SD above the mean signal of zero analyte.

Fifty patient samples comprising 14 from patients with cancer and 36 with benign disease or no detectable disease were tested. The results are shown in Table 1. Seven samples from the fourteen patients with cancer were positive by the complexed U2 snRNP B″ immunoassay, and 9 of thirty-six samples from patients free of cancer were positive.

TABLE 1
Truth Table Complexed U2 snRNP B″ Immunoassay Test

| | CANCER (+) | CANCER (−) | |
|---|---|---|---|
| Immunoassay | True Positive | False Positive | Total |
| Test (+) | (TP) 7 | (FP) 9 | Immunoassay (+) |
| | | | 16 |
| Immunoassay | False Negative | True Negative | Total |
| Test (−) | (FN) 7 | (TN) 27 | Immunoassay (−) |
| | | | 34 |
| | Total Cancer | Total Non-Cancer | Total Population |
| | 14 | 36 | 50 |

The sensitivity of the assay was calculated as TP/(TP+FN): 7/14=50%; the specificity was calculated as TN/(TN+FP): 27/36=75%; the positive predictive value was calculated as TP/(TP+FP): 7/16=44%; the negative predictive value was calculated as TN/(TN/FN): 27/34=79%; and the diagnostic accuracy was calculated as TP+TN/(TP+TN+FP+FN): 34/50=68%.

### Example 3

### Purification and Screening Method for U2 snRNA

This Example shows that it is possible to detect U2 snRNA in a sample using an antibody that binds specifically to the 2,2,7, trimethylguanosine CAP.

The serum samples used in this Example required no extensive pretreatment, but were diluted in a mild salt and detergent solution (1:10 “CSK” buffer: 10 mM NaCl, 30 mM sucrose, 1 mM PIPES pH 6.8, 500 μM MgCl₂, 0.05% Triton X-100) at a mixture of not less than 1:1 with the 1:10 CSK buffer. In addition, 10 μL of RNAse inhibitor (Ambion Inc., Austin, Tex., Catalog number 2682) was added to each sample.

A separate capture column was prepared for each sample. The resin used to prepare each capture column contained 2,2,7-trimethylguanosine agarose-linked conjugate from Oncogene Science (Catalog number NA02A). The resin was placed in a polypropylene centrifuge filter apparatus (for example, Pierce EZ Kit catalog number 4051742). The amount of resin was 50 μg, but other amounts are contemplated. The resin column was washed three times with 400 μL of coupling buffer before use (200 mM ammonium acetate with 16 μL of RNASecure™ (Ambion catalog number 7005)). A 1/25 volume of RNASecure™ was added to all column washes. The washes consisted of direct addition of the wash solution to the column bed followed by centrifugation for 1 minute on a tabletop micro-centrifuge (for example the National Labnet Company model C-1200) at 1000 revolutions per minute. The wash was discarded from the collection tube of the column apparatus. The collection tube was reused until the product was eluted.

400 μL of diluted sera was added to each of the prepared columns. Each column was allowed to tumble 1 hour at room temperature on a benchtop test tube rotary rocker (Barnstead/Thermolyne model 400110) at a rotation frequency of 8 revolutions per minute. The void volume of liquid was removed by repeating the centrifugation step into the collection tube. This volume, referred to as the “flow through,” was used for analysis of the protocol. The column was washed as indicated above three times using 400 μL of 100 mM ammonium acetate pH 7. The washes were collected for analysis.

The captured RNA then was eluted by adding 200 μL of elution buffer (15 mM 7-methyl guanosine in a solution of 300 mM ammonium chloride). The apparatus was allowed to tumble for ten minutes at room temperature. The apparatus was centrifuged as described above in the wash steps and the eluate collected for diagnostic analysis.

The column can be regenerated, although regeneration is not presently preferred when performing diagnostic tests. To regenerate the column, 200 μL of low pH buffer (0.1 M glycine-HCl pH 2.45) were added. The column was allowed to tumble as in the wash and elution steps and the volume can be collected for analysis of the protocol. The column is washed with 400 μL of standard PBS as with the previous wash steps. 400 μL of 1.4 M NaCl were added and the column was stored at 4° C.

RT-PCR was performed on the diagnostic eluate using the Titanium™ One-Step RT-PCR Kit (Clontech/BD Biosciences catalog number 639504 K1403) and the RT-PCR primers of SEQ ID NO. 86 and SEQ ID NO. 87. RNA-primer mixtures for 75° C. heat treatment were prepared. The primer mixtures were each 7.5 μL total and included 1 μL of primers (45 μM), 1 μL of RNA sample or control, and Kit RT buffer (RNAse inhibitor, GC melt dNTP's, RT). The heat treatment program on the MJ 100 Thermal Cycler was run. 42.5 μL of PCR Master Mix were added to each tube; Kit PCR buffer with Taq enzyme was used. The Thermal Cycler Program “2-Step” was run with the following parameters: 50° C.×1 hour; 95° C.×5 minutes; 95° C.×30 seconds; 58° C.×30 seconds; repeat 35 times; 68° C.×2 minutes; hold at 4° C.

Samples were subsequently evaluated by gel electrophoresis. U2 snRNA abundance could be determined by examination of the gel image and threshold dilution analysis. It is contemplated that RT-PCR can also be used to measure the product. An example of such a gel appears in FIG. 5.

Fifty patient samples comprising 14 from patients with cancer and 36 with benign disease or no detectable disease were tested with an RT-PCR assay for U2 snRNA. The results are shown in Table 2. RT-PCR detected the presence of U2 snRNA in 30 of the samples. U2 snRNA was amplified in thirteen of the fourteen samples from cancer patients and in 19 of 36 samples from patients with benign or no detectable disease.

TABLE 2
Truth Table for PCR Assay (U2 snRNA)

| | CANCER (+) | CANCER (−) | |
|---|---|---|---|
| PCR Test (+) | True Positive | False Positive | Total |
| | (TP) 13 | (FP) 17 | PCR (+) |
| | | | 30 |
| PCR Test (−) | False Negative | True Negative | Total |
| | (FN) 1 | (TN) 19 | PCR (−) |
| | | | 20 |
| | Total Cancer | Total | Total |
| | 14 | Non-Cancer | Population |
| | | 36 | 50 |

The sensitivity for the assay was calculated as TP/(TP+FN): 13/14=93%; the specificity was calculated as TN/(TN+FP): 19/36=53%; the positive predictive value was calculated as TP/(TP+FP): 13/30=43%; the negative predictive value was calculated as TN/(TN+FN): 19/20=95%; and the diagnostic accuracy was calculated as TP+TN/(TP+TN+FP+FN): 32/50=64%;

### Example 4

### Morpholino Purification and Screening Method

This Example provides a protocol for capturing U2 particles.

The affinity resin was prepared as follows. U2 specific morpholinos, as set forth in SEQ ID NO. 88, with a primary amine synthesized at the 3′ end (GeneTools, LLC Philomath OR) were obtained in 300 nmol amounts. The morpholinos were immobilized on agarose-4CLB using the AminoLink® Immobilization Kit and AminoLink® Coupling Gel (Pierce Biotechnology, Inc., Rockford, Ill.) in accordance with the manufacturer's instructions.

Serum samples were diluted in a mild salt and detergent solution (e.g., 10 mM NaCl, 30 mM sucrose, 1 mM PIPES pH 6.8, 0.5 mM MgCl₂, 0.05% Triton X-100) at a mixture of not less than 1:1. In addition, 10 μL of RNAse inhibitor (Ambion Inc. Austin Tex. Catalog # 2682) were added to each sample.

Then, each sample was pre-cleared using a column packed with Agarose-4Clb (Amersham Pharmacia). The pre-clearing was performed on at least 1.0 mL of diluted sample. The vessel used for performing the pre-clearing was a vial or a tube made of unwettable material that had a capacity of at least 5 times the volume of the sample and resin to be used in the pre-clearing. An amount of resin 6 times greater than the sample was selected. The pre-clearing column matrix (agarose 4clb) agarose resin was washed with an equal volume of WB 250 buffer (250 mM NaCl, 20 mM HEPES, pH 7.9, 0.05% NP-40, 5 mM PMSF, 0.5 mM DTT). The resin was centrifuged using a table-top centrifuge (National Labnet Company model C-1200) at 1000 g for 5 minutes, and the supernatant buffer was removed. An equal volume of fresh WB250 then was added. The resulting beads were divided into five aliquots, each aliquot corresponding to at least 0.6 volumes of the total amount of diluted sample to be pre-cleared. The aliquots were centrifuged as before and the supernatant buffer removed from each resin. The serum sample to be tested, diluted 1:1 with WB250, was added to one of the aliquots. The sample and resin mix was slowly rotated for one hour. Subsequently, the sample and resin mix was centrifuged at 1000 g for 2 minutes and the supernatant removed. The supernatant was added to a fresh aliquot of WB250 washed agarose. The steps of rotation mixing, centrifugation and supernatant transfer were repeated three more times. The final supernatants were designated “pre-cleared extracts” or PCEs, and were analyzed immediately or frozen at minus 80° C.

70 μL of premix was prepared containing the following components: 15 μL of 0.1M ATP, 10 μL of 0.5M creatine phosphate, 2 μL of 1M MgCl₂, complete with H₂O to final volume of 70 μL, 150 μl of affinity resin. 70 μL premix was added to 0.9 mL of PCE and mixed gently. 30 μL of 5M NaCl was added to bring the PCE mixture to a final concentration of 250 mM NaCl. The mixture was incubated at 30° C. for 2.5 hours at room temperature. The extract then was centrifuged at 1300 g. The supernatant was removed and the residual gel was washed 5 times with 200 μL of WB 100 (the composition was identical to WB250, with the exception that the concentration of NaCl was 100 mM). The gel then was washed 5 times with 200 μL of WB250. The U2 particles were first eluted by incubation at 75° C., 10 minutes in WB100 (E1). The remaining U2 particles were eluted with 200 μL water (E2). The elutes were centrifuged and the supernatants collected. The collected samples were concentrated to dryness in Speed-Vac (Savant) vacuum evaporator. The pellets then were re-suspendended in an appropriate buffer for analysis (eg., PCR, ELISA).

It is contemplated that resulting E1 and E2 fractions can be used for diagnostic purposes. For example, the E1 fraction may be used in quantitative RT-PCR measurements, or other nucleic acid-based measurements. The E2 fraction may be used in protein measurements including immunoassays such as ELISAs. The results from nucleic acid and/or protein methods may be used to diagnose cancer in an individual.

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced by reference therein.

Incorporation By Reference

The entire disclosure of each of the aforementioned patent and scientific documents cited hereinabove is expressly incorporated by reference herein for all purposes.

1. A method of diagnosing cancer in a mammal, the method comprising the steps of:
(a) disrupting a complex in a tissue or body fluid sample from a mammal, the complex comprising one or more components of spliceosomal particle U2; and
(b) detecting a component of spliceosomal particle U2, wherein the presence of the component is indicative of the presence of cancer in the mammal.

2. A method of diagnosing cancer in a mammal, the method comprising the steps of:
(a) disrupting a complex in a tissue or body fluid sample from a mammal, the complex comprising one or more components of spliceosomal particle U2; and
(b) measuring the amount of the component of spliceosomal particle U2, wherein the amount of the component is indicative of the presence of cancer in the mammal.

3. The method of claim 1 or 2, wherein the complex is disrupted by mixing the complex with a denaturant.

4. The method of claim 3, wherein the denaturant is urea.

5. A method of diagnosing cancer in a mammal, the method comprising the step of detecting in a tissue or body fluid sample isolated from the mammal the presence of a component of spliceosomal particle U2, which if present is indicative of cancer in the mammal, provided that the component is not U2 snRNP B″.

6. A method of diagnosing cancer in a mammal, the method comprising the step of measuring in a tissue or body fluid sample isolated from the mammal an amount of a component of spliceosomal particle U2, wherein the amount is indicative of cancer in the mammal, provided that the component is not U2 snRNP B″.

7. The method of claim 2 or 6, wherein the amount, when greater than or equal to a threshold value, is indicative of the presence of cancer in the mammal.

8. A method of diagnosing cancer in a mammal, the method comprising the steps of:
(a) combining a tissue or body fluid sample isolated from the mammal with a purified binding moiety capable of binding specifically to a component of spliceosomal particle U2 thereby to form a complex comprising the binding moiety and the component, provided that the component is not U2 snRNP B″; and
(b) detecting the presence of the complex, which, if present, is indicative of the presence of cancer in the mammal.

9. A method of diagnosing cancer in a mammal, the method comprising the steps of:
(a) combining a tissue or body fluid sample isolated from the mammal with a purified binding moiety capable of binding specifically to a component of spliceosomal particle U2 thereby to form a complex comprising the binding moiety and the component, provided that the component is not U2 snRNP B″; and
(b) measuring the amount of the complex, wherein an amount of the complex greater than or equal to a threshold value is indicative of the presence of cancer in the mammal.

10. The method of claim 1 or 2, wherein the component is U2 snRNP B″.

11. The method of claim 5 or 6, wherein the component is U2 snRNA.

12. The method of claim 5 or 6, wherein the component is SAP155.

13. The method of claim 5 or 6, wherein the component is SAP145.

14. The method of claim 5 or 6, wherein the component is SPF31.

15. The method of claim 5 or 6, wherein the component is selected from the group consisting of SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30.

16. The method of claim 11, wherein the detecting step comprises amplifying the U2 snRNA.

17. The method of claim 5 or 6, wherein the detecting or measuring step comprises detecting or measuring the amount of a plurality of components of the U2 spliceosomal particle.

18. The method of claim 5 or 6, wherein the detecting or measuring step comprises detecting or measuring a second, different component of the U2 spliceosomal particle.

19. The method of claim 18, wherein the second, different component is selected from the group consisting of SAP155, SAP145, SPF31, SAP130, SAP114, SAP62, SAP61, SAP49, U2 snRNP A′, p14, U2AF35, U2AF65, U2AF1-RS2, hPrp5p, hPrp19, HuR, ALY, SR140, CHERP, hPrp43, HSP75, PUF60, Hsp60, SPF45, BRAF35, SF2/ASF, SF3b14b, SF3b10, SF3a120, SF3a66, SF3a60, and SPF30.

20. The method of claim 18, wherein the second, different component is selected from the group consisting of U2 snRNP B″ and U2 snRNA.

21. The method of claim 8 or 9, wherein the binding moiety is selected from the group consisting of a nucleic acid, a nucleic acid analog, and a protein.

22. The method of claim 21, wherein the protein is a snurportin protein.

23. The method of claim 21, wherein the protein is an antibody or an antigen-binding fragment thereof.

24. The method of claim 23, wherein the antibody is selected from the group consisting of an anti-2,2,7-trimethylguanosine antibody, an anti-Sm antibody, an anti-SMN antibody, an anti-Importin B antibody, an anti-snurportin antibody, an anti-Ran antibody, and an anti-Ran-GTP antibody.

25. The method of claim 5, wherein the detecting step comprises mass spectrometry.

26. The method of claim 2, 6, and 9, wherein the amount is a relative amount.

27. A method of detecting one or more snRNAs comprising 2,2,7-trimethylguanosine in a body fluid sample isolated from a mammal, the method comprising:
(a) contacting the sample with a binding moiety that specifically binds 2,2,7-trimethylguanosine, such that, if an snRNA comprising 2,2,7-trimethylguanosine is present in the sample, the snRNA binds to the moiety to produce a complex; and
(b) detecting the presence, absence or amount of the complex.

28. The method of claim 27, wherein the presence or amount of the complex is indicative of the presence of cancer.

29. The method of claim 27 or 28, wherein the binding moiety is an antibody or an antigen-binding fragment thereof.

30. The method of claim 5 or 6, wherein the mammal is a human.

31. The method of claim 5 or 6, wherein the sample is a breast tissue sample.

32. The method of claim 5 or 6, wherein the cancer is breast cancer.

33. The method of claim 5 or 6, wherein the sample is a body fluid sample selected from the group consisting of blood, serum, plasma, nipple aspirate, ductal lavage fluid, fine needle aspirate, sweat, tears, urine, peritoneal fluid, lymph, vaginal secretions, semen, spinal fluid, ascitic fluid, saliva and sputum.

34. A kit comprising:
(a) a purified binding moiety that specifically binds 2,2,7-trimethylguanosine; and
(b) one or more molecules complementary to at least a portion of a U2 snRNA.

35. The kit of claim 34, wherein the binding moiety is an antibody or an antigen-binding fragment thereof.

36. A kit for detecting cancer, the kit comprising:
(a) a purified binding moiety that specifically binds U2 snRNA; and
(b) a reference sample having an amount of U2 snRNA indicative of the presence of cancer.

37. The kit of claim 36, further comprising a receptacle for receiving a sample from a patient.

38. The kit of claim 36, wherein the binding moiety is a nucleic acid or nucleic acid analog complementary to at least a portion of the U2 snRNA.

39. The kit of claim 36, wherein the binding moiety is an antibody or an antigen-binding fragment thereof.
